(19) United States
(12) Patent Application Publication
(10) Pub. No.: US 2019/0060479 A1
(43) Pub. Date: Feb. 28, 2019

(54) POLYMALIC ACID BASED NANOIMMUNOCONJUGATES AND USES THEREOF

(71) Applicant: CEDARS-SINAI MEDICAL CENTER, Los Angeles, CA (US)

(72) Inventors: Julia Y. LJUBIMOVA, Studio City, CA (US); Keith L. BLACK, Los Angeles, CA (US); Eggehard HOLLER, Los Angeles, CA (US); Alexander LJUBIMOV, Studio City, CA (US); Hui DING, Los Angeles, CA (US)

(73) Assignee: CEDARS-SINAI MEDICAL CENTER, Los Angeles, CA (US)

(21) Appl. No.: 16/081,584

(22) PCT Filed: Mar. 3, 2017
(86) PCT No.: PCT/US17/20666

§ 371 Date: Aug. 31, 2018

Related U.S. Application Data
(60) Provisional application No. 62/303,845, filed on Mar. 4, 2016.

(51) Int. Cl. A61K 47/68; A61K 47/59; A61K 47/18; A61K 47/10; A61K 45/06; A61P 35/00

(52) U.S. Cl. CPC A61K 47/6851; A61K 47/6883; A61K 47/593; A61K 31/7105; A61K 47/10; A61K 45/06; A61P 35/00; A61K 47/183

(57) ABSTRACT

Nanoimmunoconjugates including a polymalic acid-based molecular scaffold, targeting ligands, anti-tumor immune response stimulators and anti-cancer agents are provided. Methods for treating cancer in a subject by administering the nanoimmunoconjugates that provide both systemic and local immune responses and synergistic anticancer effect are described.

### CROSS REFERENCE TO RELATED APPLICATION

This application claims the benefit of U.S. provisional application No. 62/303,845, filed Mar. 4, 2016, which is incorporated by reference as if fully set forth.

### STATEMENT REGARDING FEDERALLY SPONSORED RESEARCH OR DEVELOPMENT

The invention was made with government support under Grant No. CA206220-01 awarded by National Institutes of Health. The government has certain rights in the invention.

The sequence listing electronically filed with this application titled “Sequence Listing,” which was created on Mar. 3, 2017 and had a size of 1,889 bytes is incorporated by reference herein as if fully set forth.

### FIELD OF INVENTION

The present disclosure generally relates to compositions and methods for treating patients having cell proliferative disorders with polymalic acid-based nanoimmunoconjugates that can provide both systemic and local immune response and provide synergistic anticancer effect.

### BACKGROUND

Breast cancer is the most diagnosed malignancy and the second cause of cancer death in women in the United States. In 2015 over 233,000 new cases of breast cancer will be diagnosed and approximately 40,000 women are projected to die from breast cancer in the United States. Despite advancements in early diagnosis and new therapies, relapse is still a major problem in breast cancer patients, and once the disease becomes metastatic it is extremely challenging to cure. Breast cancer primarily metastasizes in regional lymph node, bone, lungs, liver, and brain. Brain metastasis is observed in 10-15% of breast cancer patients and is particularly difficult to treat. Unfortunately, a significant number of breast cancer patients never respond to this therapy and those who respond acquire resistance and die. Thus, new therapies for breast cancer patients are still urgently needed.

The National Cancer Institute estimates that 22,850 malignant brain and spinal cord tumors will be diagnosed in 2015 in the U.S. Gliomas are the most common brain malignancies, and a very aggressive tumor, glioblastoma grade IV (glioblastoma multiforme, or GBM), is the most frequent. In spite of huge effort and a wealth of new data on glioma biology, the patients' survival did not significantly change in the last 25 years. Therefore, there is an unmet clinical need in uncovering glioma molecular markers and in developing efficient ways of modulating their expression through targeted drug delivery specifically into brain tumors.

Progress in treatment of primary cancers has led to increased patients' longevity but has also increased the chance of residual tumor cells to metastasize, in particular to the brain. Little progress in pharmacological brain cancer treatment is largely due to the inability of many drugs to cross the blood-brain barrier (BBB) formed by brain vascular endothelium. For instance, clinically used therapeutic monoclonal antibodies (mAbs) are effective for primary tumor treatment but cannot penetrate BBB to reach brain tumors. Another obstacle in brain tumor treatment is brain immune privilege hampering efficient immunotherapy.

Despite the progress in immunotherapy of tumors such as melanoma, lung, and prostate cancers, surprisingly little is known about the role of the immune system in human breast and brain cancer development, as compared to other cancers.

### SUMMARY

In an aspect, the invention relates to a nanoimmunoconjugate that comprises a polymalic acid-based molecular scaffold, at least one targeting ligand, at least one anti-tumor immune response stimulator and at least one anti-cancer agent. The targeting ligand, the anti-tumor immune response stimulator and the anti-cancer agent are covalently linked to the polymalic acid-based molecular scaffold.

In an aspect, the invention relates to a method for treating cancer in a subject. The method comprises providing any one of the nanoimmunoconjugates described herein. The method also comprises administering a therapeutically effective amount of the nanoimmunoconjugates to a subject.

In an aspect, the invention relates to a pharmaceutically acceptable composition comprising any one of the nanoimmunoconjugates described herein and a pharmaceutically acceptable carrier or excipient.

In an aspect, the invention relates to a method for treating cancer in subject. The method comprises providing a nanoconjugate comprising a polymalic acid-based molecular scaffold and at least one targeting ligand and at least one anti-cancer agent covalently linked to the scaffold. The method also comprises co-administering a therapeutically effective amount of an anti-tumor immune response stimulator and a therapeutically effective amount of the nanoconjugate to a subject.

### BRIEF DESCRIPTION OF THE DRAWINGS

The following detailed description of the preferred embodiments will be better understood when read in conjunction with the appended drawings. For the purpose of illustration, there are shown in the drawings embodiments which are presently preferred. It is understood, however, that the invention is not limited to the precise arrangements and instrumentalities shown. In the drawings:

FIG. 1 is a schematic drawing illustrating an exemplary nanoimmunoconjugate (NIC) that includes a PMLA backbone (P), mPEG 5000 for stability, an endosomal escape unit (LLL), an anti-TfR mAb for BBB and breast tumor targeting, and an AON against CK2 to induce tumor cytotoxicity and mechanism of action of nanoimmunoconjugates (NIC) in the context of breast cancer.

FIG. 2 is a set of line graphs illustrating anti-tumor activity of NIC (P/mPEG/LLL/mTfR/IL-2; x-mark) in a human xenograft breast cancer (BT-474) model compared to control treatments with PBS (closed diamond) and P/IL-2 (closed square).

FIG. 3 is a set of line graphs illustrating anti-tumor activity of nanoimunnoconjugates P/CTLA-4/IgG (closed triangle) and P/CTLA-4/MsTfR (x-mark) in BALB/c mice bearing s.c. D2F2 syngeneic mammary tumors in comparison to control treatments with PBS (closed diamond) and CTLA-4 mAb (closed square).

FIGS. 4A-4B are sets of bar graphs illustrating preferential IL-12 (FIG. 4A) and IL-10 (FIG. 4B) activation induced by anti-CTLA-4 in BALB/c mice with s.c. D2F2 syngeneic mammary tumors. FIG. 4A illustrates IL-12 activation induced by P/IgG/CTLA-4, P/mTfR/CTLA-4, CTLA-4 in comparison to control treatments with serum and PBS. FIG. 4B illustrates IL-10 activation induced by P/IgG/CTLA-4, P/mTfR/CTLA-4, CTLA-4 in comparison to control treatments with serum and PBS.

FIGS. 5A-5B are sets of bar graphs illustrating immunostimulation in animals with intracranial D2F2 tumors (brain metastatic model). FIG. 5A illustrates IL-12 activation induced by P/IgG/CTLA-4, P/mTfR/CTLA-4, CTLA-4 in comparison to control treatments with serum and PBS. FIG. 5B illustrates IL-10 activation induced by P/IgG/CTLA-4, P/mTfR/CTLA-4, CTLA-4 in comparison to control treatments with serum and PBS.

FIG. 6 is a set of Kaplan-Meier survival curves for BALB/c mice bearing intracranial mammary D2F2 tumors (brain metastatic model) after treatment with P/mPEG/LLL/mTfR/CTLA-4, anti-CTLA-4 Ab and PBS.

FIG. 7 illustrates the synthesis of an exemplary PMLA NIC containing 40% LLL, 2% mPEG, 0.2% mTfR Ab, 0.2% CTLA4 mAb, 0.4% IL-2, and 2% Morpholino AON-HER2/neu.

FIG. 8 is a photograph of Western blot showing CK2α and β-tubulin expression in human breast cancer BT-474, mouse breast cancer D2F2 and normal human breast tissue.

FIG. 9 is a set of Kaplan Meier survival curves illustrating human brain glioma LN229 growth inhibition by nanoconjugate P/Cetu/CK22a crossing BBB and blocking CK2α in a xenogeneic animal model in comparison to control treatment with PBS.

FIG. 10 is a set of photographs illustrating expression of cancer stem cell markers CD133 and c-Myc in BT-474 HER2/neu positive i.c. tumors (brain metastatic model) treated with P/trastuzumab/MsTfR-mAb/HER2-AON and PBS.

FIG. 11 is a schematic drawing illustrating effects of a nanoimmunoconjugate that includes a PMLA backbone, LLL, a TfR mAb, a-CTLA-4 (PD-1), AON-CK2, and AON-EGFR on brain tumors.

FIGS. 12A-12B are schematic drawings of the PMLA-based nanoimmunoconjugates designed for syngeneic mouse models. FIG. 12A illustrates a nanoimmunoconjugate containing a PMLA-backbone, LLL, mPEG, CTLA-4(PD-1) mAB, msTfR mAb, AON-EGFR, AON-CK2, and optionally Alexa Fluor 680 dye designed for suppression of tumor cell growth by blocking EGFR and CK2 with AON. FIG. 12B illustrates an immunostimulatory nanoimmunoconjugate containing a PMLA-backbone, LLL, mPEG, CTLA-4(PD-1) mAB, msTfR mAb with attached active cytokine (IL-2) for additional immune stimulation and optionally Alexa Fluor 680 dye.

FIGS. 13A-13B are photographs of Western blots showing EGFR and CK2α expression in GBMs and their inhibition by nanodrug-conjugated AONs. FIG. 13A illustrates that both EGFR and CK2α are expressed in three cell lines U87MG, LN229, and GL26. FIG. 13B illustrates that compared to PBS, the expression of EGFR and CK2α is markedly reduced upon cell treatment with P/Cetu/AON-EGFR (left panel) and P/Cetu/AON-CK2α (right panel) using anti-EGFR mAb cetuximab (Cetu) for cellular uptake.

FIG. 14 illustrates the synthesis of an exemplary nanoimmunoconjugate that contains a PMLA backbone, 40% LLL, 2% mPEG, 0.2% TfR Ab, 0.2% CTLA-4/PD-1 Ab, 1% AON-EGFR, and 1% AON-CK2α.

FIGS. 15A-15D illustrate selective cleavage of a PMLA nanoimmunoconjugate. FIG. 15A is a schematic drawing of selective cleavage of the PMLA nanoconjugate by ammonia. FIG. 15B is an HPLC profile of the PMLA nanoimmunoconjugate before (upper curve) and after cleavage (lower curve). FIG. 15C is a graph identifying the first peak as mAb with maximum spectrum wavelength of 280 nm. FIG. 15D is a graph identifying the second peak as AON at 260 nm.

FIGS. 16A-16B illustrate that nanoimmunoconjugates containing AONs specific to EGFR and/or CK2α inhibit LN229 GBM growth and prolong tumor-bearing animal survival. FIG. 16A (left) is a set of Kaplan-Meier curves showing significantly increased survival upon treatment with nanoimmunoconjugates P/Cetu/AON-CK2α (closed square), P/Cetu/AON-EGFR and P/Cetu/AON-CK2α/AON-EGFR compared to control treatment with PBS (x-mark), and (right) is a table showing quantitation of median survival. FIG. 16B are photographs of tumor morphology following treatments with nanoimmunoconjugates and PBS

FIGS. 17A-17E illustrate effects of nanoimmunoconjugates P/Cetu/AON-CK2α, P/Cetu/AON-EGFR, and P/Cetu/AON-EGFR/AON-CK2α on pro-survival and proliferative signaling in intracranial LN229 xenogeneic tumors compared to control treatment with PBS. FIG. 17A is a set of photograph of Western blots showing reduction of EGFR, CK2α, as well as of phosphorylated/activated Akt (pAkt) and c-Myc in treated tumors. FIG. 17B is set of bar graphs showing relative expression levels of EGFR in treated tumors. FIG. 17C is set of bar graphs showing relative expression levels of CK2α in treated tumors. FIG. 17D is set of bar graphs showing relative expression levels of pAkt/Akt in treated tumors. FIG. 17E is set of bar graphs showing relative expression levels of cMyc in treated tumors.

FIG. 18 is a set of photographs illustrating expression of cancer stem cell markers CD133, cMyc and Nestin in GL26 brain tumors following treatment with P/AON-CK2α, P/AON-EGFR, P/AON-EGFR/AON-CK2α and PBS.

FIGS. 19A-19B is a set of Kaplan Meier curves illustrating animal survival after treatment with nanoimmunoconjugates. FIG. 19A illustrates animal survival after treatments with CTLA-4 mAb, P/TfR/CTLA-4 mAb and a combination of P/TfR/CTLA-4 and P/TfR/PD-1. FIG. 19B illustrates animal survival after treatments with PD-1 mAB, P/TfR/PD-1 mAb and a combination of P/TfR/CTLA-4 and P/TfR/PD-1.

FIG. 20 is a photograph illustrating delivery of the nanoimmunoconjugate P/a-CTLA-4/PD-1/TfR to the animal brain through BBB following I.V. administration.

FIG. 21 is a scatter plot illustrating analysis of IFNγ/CD8+ cells following treatments of animals with CTLA-4 mAb, P/msTfR/CTLA-4 and P/msTfR/CTLA-4+P/msTfR/PD-1.

FIG. 22 is a scatter plot illustrating analysis of CD69+/CD8+ cells following treatments of animals with CTLA-4 mAb, P/msTfR/CTLA-4 and P/msTfR/CTLA-4+P/msTfR/PD-1.

FIGS. 23A-23C are bar graphs illustrating cytokine levels in serum from C57/BI6 mice bearing GL26 glioma following treatments with P/msTfR/CTLA-4, P/msTfR/PD-1 and P/msTfRCTLA-4+P/msTfR/PD-1. FIG. 23A illustrates IL-12(p70) levels. FIG. 23B illustrates IFNγ levels. FIG. 23C illustrates TNFα levels.

### DETAILED DESCRIPTION OF PREFERRED EMBODIMENTS

Certain terminology is used in the following description for convenience only and is not limiting. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise.

The phrase “at least one” followed by a list of two or more items, such as “A, B, or C,” means any individual one of A, B or C as well as any combination thereof.

The words “right,” “left,” “top,” and “bottom” designate directions in the drawings to which reference is made.

Although methods and materials similar or equivalent to those described herein can be used in the practice or testing of this disclosure, suitable methods and materials are described below.

The terms “proliferative disorder” and “proliferative disease” refer to disorders associated with abnormal cell proliferation such as cancer.

The terms “tumor” and “neoplasm” as used herein refer to any mass of tissue that result from excessive cell growth or proliferation, either benign (noncancerous) or malignant (cancerous) including pre-cancerous lesions.

The term “primary cancer” refers to the original site at which a cancer originates. For example, a cancer originating in the breast is called a primary breast cancer. If it metastasizes, i.e., spreads to the brain, the cancer is referred to as a primary breast cancer metastatic to the brain.

The term “metastasis” as used herein refers to the process by which a cancer spreads or transfers from the site of origin to other regions of the body with the development of a similar cancerous lesion, i.e., having the same or substantially the same biochemical markers at the new location. A “metastatic” or “metastasizing” cell is one that has a reduced activity for adhesive contacts with neighboring cells and migrates by the bloodstream or within lymph from the primary site of disease to additional distal sites, for example, to invade neighboring body structures or distal structures.

The terms “cancer cell”, “tumor cell” and grammatical equivalents refer to a cell derived from a tumor or a pre-cancerous lesion including both a non-tumorigenic cell and a tumorigenic cell, i.e., cancer stem cell.

As used herein “tumorigenic” refers to the functional features of a solid tumor stem cell including the properties of self-renewal, i.e., giving rise to additional tumorigenic cancer cells, and proliferation to generate other tumor cells, i.e., giving rise to differentiated and thus non-tumorigenic tumor cells, such that cancer cells form a tumor.

The term “antibody” is used herein to mean an immunoglobulin molecule that is a functional module included in compositions herein for ability to recognize and specifically bind to a target, such as a protein, polypeptide, peptide, carbohydrate, polynucleotide, lipid, or combinations of the foregoing through at least one antigen recognition site within the variable region of the immunoglobulin molecule. In an embodiment, antibodies included as functional modules of compositions herein may include a class described as antagonist antibodies, which specifically bind to a cancer stem cell marker protein and interfere with, for example, ligand binding, receptor dimerization, expression of a cancer stem cell marker protein, and/or downstream signaling of a cancer stem cell marker protein. In alternative embodiments, antibodies as functional modules in compositions herein include agonist antibodies that specifically bind to a cancer stem cell marker protein and promote, for example, ligand binding, receptor dimerization, and/or signaling by a cancer stem cell marker protein. In alternative embodiments, antibodies that do not interfere with or promote the biological activity of a cancer stem cell marker protein instead function to inhibit tumor growth by, for example, antibody internalization and/or recognition by the immune system.

As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody includes any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. Antibodies can be naked or conjugated to other molecules such as toxins, radioisotopes, etc. In other embodiments an antibody is a fusion antibody.

As used herein, the term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multispecific antibodies formed from antibody fragments.

An “Fv antibody” refers to the minimal antibody fragment that contains a complete antigen-recognition and -binding site either as two-chains, in which one heavy and one light chain variable domain form a non-covalent dimer, or as a single-chain (scFv), in which one heavy and one light chain variable domain are covalently linked by a flexible peptide linker so that the two chains associate in a similar dimeric structure. In this configuration the complementarity determining regions (CDRs) of each variable domain interact to define the antigen-binding specificity of the Fv dimer. Alternatively a single variable domain (or half of an Fv) can be used to recognize and bind antigen, although generally with lower affinity.

A “monoclonal antibody” as used herein refers to homogenous antibody population involved in specific recognition and binding of a single antigenic determinant, or epitope. Polyclonal antibodies include a population of antibody species each directed to a different antigenic determinant. The term “monoclonal antibody” encompasses both and full-length monoclonal antibodies and antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to those obtained without limitation by methods including and not limited to hybridoma expression, phage selection, recombinant expression, and by transgenic animals.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, “management” or “managing” refers to preventing a disease or disorder from occurring in a subject, decreasing the risk of death due to a disease or disorder, delaying the onset of a disease or disorder, inhibiting the progression of a disease or disorder, partial or complete cure of a disease or disorder and/or adverse effect attributable to the said disease or disorder, obtaining a desired pharmacologic and/or physiologic effect (the effect may be prophylactic in terms of completely or partially preventing a disorder or disease or condition, or a symptom thereof and/or may be therapeutic in terms of a partial or complete cure for a disease or disorder and/or adverse effect attributable to the disease or disorder), relieving a disease or disorder (i.e. causing regression of the disease or disorder). Further, the present disclosure also envisages treating the said disease by administering the therapeutic composition of the instant disclosure.

The terms “subject” and “individual” are used interchangeably herein, and mean a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In an embodiment, the subject may be a mammal, e.g., a primate, e.g., a human. The terms, “patient” and “subject” are used interchangeably herein. The terms, “patient” and “subject” are used interchangeably herein.

Preferably, the subject is a mammal. The mammal may be a human, non-human primate, mouse, rat, dog, cat, horse, or cow, but are not limited to these examples. Mammals other than humans may be advantageously used as subjects that represent animal models of cancer. In addition, the methods described herein may be used to treat domesticated animals and/or pets. A subject may be male or female. A subject may be one who has been previously diagnosed with or identified as suffering from cancer, but need not have already undergone treatment.

As used herein, the term “co-administering,” “co-administration,” or “co-administer” refers to the administration of at least two different compounds and/or compositions, wherein the compounds and/or the compositions may be administered simultaneously, or at different times, as long as they work additively or synergistically to treat cancer. Without limitations, the two different compounds and/or compositions may be administered in the same formulation or in separate formulations. When administered in separate formulations, the compounds and/or compositions may be administered within any time of each other. For example, the compounds and/or compositions may be administered within 24 hours, 12 hours, 6 hours, 5 hours, 4 hours, 3 hours, 2 hours, 1 hour, 45 minutes, 30 minute, 25 minutes, 20 minutes, 15 minutes, 10 minutes, 5 minutes or less of each other. Further, when administered in separate formulations, the compounds and/or compositions may be administered in any order. Additionally, co-administration does not require that the co-administered compounds and/or compositions be administered by the same route. As such, each may be administered independently or as a common dosage form. Further, the two compounds may be administered in any ratio to each other by weight or moles. For example, two compounds may be administered in a ratio of from about 50:1, 40:1, 30:1, 25:1, 20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:25, 1:30, 1:40, or 1:50. The ratio may be based on the effective amount of either compound.

An embodiment provides a nanoimmunoconjugate capable of simultaneous specific cancer cell killing and stimulation of anti-tumor immune response, and significantly increase anti-tumor efficacy. The nanoimmunoconjugate may comprise a polymalic acid-based molecular scaffold, at least one targeting ligand, at least one anti-tumor immune response stimulator and at least one anti-cancer agent. Each of the targeting ligand, the anti-tumor immune response stimulator and the anti-cancer agent may be covalently conjugated or linked with the polymalic acid-based molecular scaffold.

As used herein, the term “polymalic acid” refers to a polymer, e.g., a homopolymer, a copolymer or a blockpolymer that contains a main chain ester linkage. The polymalic acid may be at least one of biodegradable and of a high molecular flexibility, soluble in water (when ionized) and organic solvents (in its acid form), non-toxic, or non-immunogenic (Lee B et al., Water-soluble aliphatic polyesters: poly(malic acid)s, in: Biopolymers, vol. 3a (Doi Y, Steinbuchel A eds., pp 75-103, Wiley-VCH, New York 2002, which is incorporated herein by reference as if fully set forth). In an embodiment, the polymalic acid may be poly(β-L-malic acid), herein referred to as poly-β-L-malic acid or PMLA.

Without limitations, the polymalic acid may be of any length and of any molecular mass. The polymalic acid may have a molecular mass of 10, 20, 30, 40, 50, 60, 70, 80, 90, 95, or 100 kDa, or more. In an embodiment, the polymalic acid may have a molecular mass in a range between any two of the following molecular masses: 10, 20, 30, 40, 50, 60, 70, 80, 90, 95, or 100 kDa.

Exemplary polymalic acid-based molecular scaffolds amenable to the nanoimunoconjugates disclose herein are described, for example, in PCT Appl. Nos. PCT/US04/40660, filed Dec. 3, 2004, PCT/US09/40252, filed Apr. 10, 2009, and PCT/US10/59919, filed Dec. 10, 2010, PCT/US10/62515, filed Dec. 30, 2010; and U.S. patent application Ser. No. 10/580,999, filed Mar. 12, 2007, and Ser. No. 12/935,110, filed Sep. 28, 2010, contents of all which are incorporated herein by reference as if fully set forth.

As used here, the term “anti-tumor immune response stimulator” refers to an agent that is capable of eliciting an anti-tumor immune response. As used herein, the term “anti-tumor immune response” means an immune response directed against a tumor, tumor cell, a cancer cell, and/or antigens expressed by a tumor/cancer cell. The immune response can be T cell mediated and/or B cell mediated immune response. Exemplary immune responses include T cell responses, e.g., cytokine production and cellular cytotoxicity. In addition, the immune response can include immune responses that are indirectly affected by T cell activation, e.g., antibody production (humoral responses) and activation of cytokine responsive cells, e.g., macrophages. Thus, the immune response can be innate, humoral, cellular, or any combination thereof.

In an embodiment, the anti-tumor immune response stimulator may be an agent that totally or partially reduces, inhibits, interferes with or modulates the activity or synthesis of one or more immune checkpoint proteins. In an embodiment, the anti-tumor immune response stimulator may inhibit the activity or synthesis of one or more immune checkpoint proteins. Such agents are also referred to as “an immune checkpoint inhibitor” in the present disclosure. Without wishing to be bound by a theory, inhibition of one or more immune checkpoint proteins may block or otherwise neutralize inhibitory signaling to thereby upregulate an immune response in order to more efficaciously treat cancer.

As used herein, the term “immune checkpoint proteins” means a group of molecules on the cell surface of CD4+ and/or CD8+ T cells that fine-tune immune responses by down-modulating or inhibiting an anti-tumor immune response. Immune checkpoint proteins are well known in the art and include, but are not limited to, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPα (CD47), CD48, 2B4 (CD244), B7.1, B7.2, ILT-2, ILT-4, TIGIT, and A2aR. See, for example, WO 2012/177624, content of which is incorporated herein by reference as if fully set forth.

Exemplary agents useful for inhibiting immune checkpoint proteins may be antibodies, low molecular weight drugs, peptides, peptidemimetics, natural ligands, or derivatives of natural ligands, that can either bind and/or inactivate or inhibit immune checkpoint inhibitor proteins, or fragments thereof; as well as interfering RNA interference, antisense oligonucleotides, nucleic acid aptamers, etc. that can downregulate the expression and/or activity of immune checkpoint inhibitor nucleic acids, or fragments thereof. Exemplary agents for upregulating an immune response may be antibodies against one or more immune checkpoint proteins that block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint protein fused to the Fc portion of an antibody or immunoglobulin) that bind to its natural receptor(s); nucleic acid molecules that block immune checkpoint protein encoding nucleic acid transcription or translation; or the like. Such agents can directly block the interaction between the one or more immune checkpoint proteins and its natural receptor(s) (e.g., antibodies) to prevent inhibitory signaling and upregulate an immune response. For example, an immune checkpoint protein ligand such as a stabilized extracellular domain can bind to its receptor to indirectly reduce the effective concentration of the receptor to bind to an appropriate ligand.

In an embodiment, the anti-tumor immune response stimulator may be an anti-PD-1 or anti-CTLA-4 antibody. Without limitations, the anti-PD-1 and/or the anti-CTLA-4 antibody may be a monoclonal or polyclonal antibody. In addition, the antibody may be a humanized antibody or a chimeric antibody. In an embodiment, the anti-PD-1 and/or the anti-CTLA-4 antibody may be IgG1.

In an embodiment, the anti-tumor immune response stimulator may be an antisense oligonucleotide (AON) or an siRNA. The antisense oligonucleotide or the siRNA may comprise a sequence complementary to a sequence contained in an mRNA transcript of an immune checkpoint protein. In an embodiment, the antisense oligonucleotide may be a Morpholino antisense oligonucleotide. The antisense oligonucleotide may include a sequence complementary to a sequence contained in an mRNA transcript of a nucleic acid encoding CTLA-4. The antisense oligonucleotide may include a sequence with at least 70, 72, 75, 80, 85, 90, 91, 92, 93, 94, 95, 96, 97, 98, 99 or 100% identity to a sequence of SEQ ID NO: 4 or 5. The antisense oligonucleotide may include a sequence complementary to a sequence contained in an mRNA transcript of a nucleic acid encoding PD-1. The antisense oligonucleotide may include a sequence with at least 70, 72, 75, 80, 85, 90, 91, 92, 93, 94, 95, 96, 97, 98, 99 or 100% identity to a sequence of SEQ ID NO: 6 or 7.

In an embodiment, the anti-tumor immune response stimulator may be an immunostimulatory cytokine. As used herein, the term “immunostimulatory cytokine” refers to any compound which promotes an increase in the activity of any component of the immune system including those components forming part or being involved in cell-mediated immune response, humoral-mediated immune response and the complement system. Immunostimulatory cytokines may be, but are not limited to, IL-2, IL-12, IL-20, IL-15, IL-18, IL-24, GM-CSF, TNFα, CD40 ligand, IFNα, IFNβ, IFNγ or functionally equivalent variants thereof. In an embodiment, the immunostimulatory cytokine may be IL-2.

In an embodiment, a nanoimmunoconjugate may comprise both an inhibitor of an immune checkpoint protein and an immunostimulatory cytokine, each covalently linked independently with the polymalic acid-based molecular scaffold.

As used herein, the term “anti-cancer agent” refers to any compound (including its analogs, derivatives, prodrugs and pharmaceutical salts) or composition, which can be used to treat cancer. Anti-cancer agents may be, but are not limited to, inhibitors of topoisomerase I and II, alkylating agents, microtubule inhibitors or angiogenesis inhibitors.

In an embodiment, the anti-cancer agent may inhibit or reduce the synthesis or activity of a human epidermal growth factor receptor (EGFR/EGFRvIII and HER2) or the serine-threonine protein kinase CK2 (CK2), a master signaling regulator for cell proliferation. Without limitations, the HER protein may be at least one protein selected from the group consisting of EGFR/EGFRvIII, HER1, HER2, HER3 or HER4. The anti-cancer agent that inhibits synthesis or activity of the HER and/or CK2 protein may be selected from the group consisting of: an antisense oligonucleotide, an siRNA oligonucleotide, an antibody, a polypeptide, an oligopeptide or a low molecular weight drug.

In an embodiment, the anti-cancer agent that inhibits the synthesis or activity of the HER, EGFR and/or CK2 may be an antisense oligonucleotide or an siRNA. The antisense oligonucleotide or the siRNA may comprise a sequence complementary to a sequence contained in an mRNA transcript of HER2/neu or the CK2 protein. In an embodiment, the antisense oligonucleotide may be a Morpholino antisense oligonucleotide. The antisense oligonucleotide may include a sequence complementary to a sequence contained in an mRNA transcript of a nucleic acid encoding HER2/neu. The antisense oligonucleotide may include a sequence with at least 70, 72, 75, 80, 85, 90, 91, 92, 93, 94, 95, 96, 97, 98, 99 or 100% identity to a sequence of SEQ ID NO: 1. The antisense oligonucleotide may include a sequence complementary to a sequence contained in an mRNA transcript of a nucleic acid encoding CK2. The antisense oligonucleotide may include a sequence complementary to a sequence with at least 70, 72, 75, 80, 85, 90, 91, 92, 93, 94, 95, 96, 97, 98, 99 or 100% identity to the sequence of SEQ ID NO: 3. The antisense oligonucleotide may include a sequence with at least 70, 72, 75, 80, 85, 90, 91, 92, 93, 94, 95, 96, 97, 98, 99 or 100% identity to a sequence of SEQ ID NO: 2. The antisense oligonucleotide may include a sequence complementary to a sequence contained in an mRNA transcript of a nucleic acid encoding EGFR. The antisense oligonucleotide may include a sequence with at least 70, 72, 75, 80, 85, 90, 91, 92, 93, 94, 95, 96, 97, 98, 99 or 100% identity to a sequence of SEQ ID NO: 8.

Determining percent identity of two amino acid sequences or two nucleic acid sequences may include aligning and comparing the amino acid residues or nucleotides at corresponding positions in the two sequences. If all positions in two sequences are occupied by identical amino acid residues or nucleotides then the sequences are said to be 100% identical. Percent identity is measured by the Smith Waterman algorithm (Smith T F, Waterman M S 1981 “Identification of Common Molecular Subsequences,” J Mol Biol 147: 195-197, which is incorporated herein by reference as if fully set forth).

An embodiment comprises synthetic nucleic acids, synthetic polynucleotides, or synthetic oligonucleotides having a portion of the sequence as set forth in any one of the nucleic acids listed herein or the complement thereof. These synthetic nucleic acids, synthetic polynucleotides, or synthetic oligonucleotides may have a length in the range from 10 to full length, 10 to 15, 10 to 20, 10 to 21, 10 to 22, 10 to 23, 10 to 24, or 10 to 25, or 10, 15, 20 or 25 nucleotides. A synthetic nucleic acid, synthetic polynucleotide, or synthetic oligonucleotide having a length within one of the above ranges may have any specific length within the range recited, endpoints inclusive. The recited length of nucleotides may start at any single position within a reference sequence (i.e., any one of the nucleic acids herein) where enough nucleotides follow the single position to accommodate the recited length. The recited length may be full length of a reference sequence.

In an embodiment, the anti-cancer agent that inhibits the synthesis or activity of the HER may be an anti-HER2/neu antibody. In an embodiment, the anti-HER2/neu antibody may be Trastuzumab Herceptin®. It is noted that the anti-HER2/neu antibody may be a monoclonal or polyclonal antibody. Further, the anti-HER2/neu antibody may be a humanized antibody or a chimeric antibody.

Additional exemplary anti-cancer agents amenable to the present invention may be, but are not limited to, paclitaxel (taxol); docetaxel; germicitibine; aldesleukin; alemtuzumab; alitretinoin; allopurinol; altretamine; amifostine; anastrozole; arsenic trioxide; asparaginase; BCG live; bexarotene capsules; bexarotene gel; bleomycin; busulfan intravenous; busulfanoral; calusterone; capecitabine; platinate; carmustine; carmustine with polifeprosan implant; celecoxib; chlorambucil; cladribine; cyclophosphamide; cytarabine; cytarabine liposomal; dacarbazine; dactinomycin; actinomycin D; darbepoetin alfa; daunorubicin liposomal; daunorubicin, daunomycin; denileukin diftitox, dexrazoxane; docetaxel; doxorubicin; doxorubicin liposomal; dromostanolone propionate; Elliott's B solution; epirubicin; epoetin alfa estramustine; etoposide phosphate; etoposide (VP-16); exemestane; filgrastim; floxuridine (intraarterial); fludarabine; fluorouracil (5-FU); fulvestrant; gemtuzumab ozogamicin; goserelin acetate; hydroxyurea; ibritumomab tiuxetan; idarubicin; ifosfamide; imatinib mesylate; interferon alfa-2a; interferon alfa-2b; irinotecan; letrozole; leucovorin; levamisole; lomustine (CCNU); mechlorethamine (nitrogenmustard); megestrol acetate; melphalan (L-PAM); mercaptopurine (6-MP); mesna; methotrexate; methoxsalen; mitomycin C; mitotane; mitoxantrone; nandrolone phenpropionate; nofetumomab; LOddC; oprelvekin; pamidronate; pegademase; pegaspargase; pegfilgrastim; pentostatin; pipobroman; plicamycin; mithramycin; porfimer sodium; procarbazine; quinacrine; rasburicase; rituximab; sargramostim; streptozocin; talbuvidine (LDT); talc; tamoxifen; temozolomide; teniposide (VM-26); testolactone; thioguanine (6-TG); thiotepa; topotecan; toremifene; tositumomab; trastuzumab; tretinoin (ATRA); uracil mustard; valrubicin; valtorcitabine (monoval LDC); vinblastine; vinorelbine; zoledronate; or any mixtures thereof.

As used herein the term “targeting ligand” refers to any molecule that provides an enhanced affinity for a selected target, e.g., a cell, cell type, tissue, organ, region of the body, or a compartment, e.g., a cellular, tissue or organ compartment. Targeting ligands may be, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL or HDL ligands.

In an embodiment, the targeting ligand may target a tumorgenic cell or cancer cell. As used herein, the phrase “target a tumorigenic cell or a cancer cell” refers to delivery of a nanoimmunoconjugate to a population of tumor-forming cells within tumors, i.e., tumorigenic cells.

In an embodiment, the targeting ligand may be an antibody specific to at least vasculature protein in a cell. In an embodiment, the vasculature protein may be a transferrin receptor protein. An antibody targeting module (TfR-Ab) may bind the transferrin receptor protein and thereby achieve transcytosis through endothelium associated with BBB. Without limitations, the antibody specific to the vasculature protein may be a monoclonal or polyclonal antibody. Further, the antibody may be a humanized antibody or a chimeric antibody.

The transferrin (Tf) receptor (TfR/CD71) is a transmembrane homodimer protein involved in iron uptake and cell growth regulation. Cancer cells express TfR at levels several-fold higher (up to 100-fold higher) than normal cells. TfR overexpression is correlated with stage and prognosis in various cancers, including breast cancer. High TfR expression levels on cancer cells, its ability to internalize, and its role in cancer pathology make it an attractive target for cancer therapy. Further, TfR has been used for delivery of a wide variety of cytotoxic molecules bound to Tf or anti-TfR mAbs by receptor-mediated endocytosis into different cancer cells including breast.

The blood-brain barrier is a high resistance barrier formed by tightly joined capillary endothelial cell membranes that maintains brain homeostasis and restricts brain access of multiple molecules including therapeutic Abs targeting cancer. However, BBB expresses TfR on its endothelial cells and anti-TfR mAbs can effectively cross BBB by transcytosis, a process used for brain delivery of therapeutic drugs including those targeting cancer. These in vitro, preclinical, and clinical studies show the efficacy and safety of targeting TfR to deliver therapeutic agents into cancer cells and are particularly relevant for drug delivery across BBB to treat deadly breast cancer brain metastases.

In an embodiment, the targeting ligand may be a lectin or another ligand specific to the transferrin receptor. In an embodiment, the targeting ligand may be a ligand to one of any number of cell surface receptors or antigens.

The molecular scaffold and the components covalently linked with the polymalic acid-based molecular scaffold may be linked to each other via a linker. As used herein, the term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroaryl alkynyl, alkenylheteroarylalkyl, alkenylheteroarylalkenyl, alkenylheteroaryl alkynyl, alkynylheteroarylalkyl, alkynylheteroarylalkenyl, alkynylheteroaryl alkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R¹ is hydrogen, acyl, aliphatic or substituted aliphatic.

In an embodiment, the linker may comprise a polyethylene glycol (PEG). Without limitations, the PEG may be of any desired molecular weight. In an embodiment, the PEG may have a molecular weight of about 1,000 Da, about 1,500 Da, about 1,000 Da, about 2,500 Da, about 3,000 Da, about 3,500 Da, about 4,000 Da, about 4,500 Da, about 5,000 Da, about 10,000 Da, about 15,000 Da, about 20,000 Da, about 25,000 Da, or about 30,000 Da. In an embodiment, the PEG may have a molecular weight of about 3,400 Da.

In an embodiment, the nanoimmunoconjugate may further comprise a PK modulating ligand covalently linked with the polymalic acid-based molecular scaffold. As used herein, the terms “PK modulating ligand” and “PK modulator” refers to molecules which can modulate the pharmacokinetics of the nanoimmunoconjugate. For example, the PK modulator can inhibit or reduce resorption of the nanoimmunoconjugate by the reticuloendothelial system (RES) and/or enzyme degradation.

PEGylation is generally used in drug design to increase the in vivo half-life of conjugated proteins, to prolong the circulation time, and enhance extravasation into targeted solid tumors (Arpicco et al., 2002 Bioconjugate Chem 13:757 and Maruyama et al., 1997 FEBS Letters 413:1771, which is incorporated herein by reference as if fully set forth). Thus, in an embodiment, the PK modulator may be a PEG. Without limitations, the PEG may be of any desired molecular weight. In an embodiment, the PEG may have a molecular weight of about 1,000 Da, about 1,500 Da, about 1,000 Da, about 2,500 Da, about 3,000 Da, about 3,500 Da, about 4,000 Da, about 4,500 Da, about 5,000 Da, about 10,000 Da, about 15,000 Da, about 20,000 Da, about 25,000 Da, or about 30,000 Da. In an embodiment, the PK modulator may be PEG of about 5,000 Da. Other molecules known to increase half-life may also be used as PK modulators.

In an embodiment, the nanoimmunoconjugate may further comprise an endosomolytic ligand covalently linked with the polymalic acid-based molecular scaffold. As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), golgi apparatus, microtubule, peroxisome, or other vesicular bodies within the cell, to the cytoplasm of the cell. The endosomolytic ligands may be, but are not limited to, imidazoles, poly or oligoimidazoles, linear or branched polyethyleneimines (PEIs), linear or branched polyamines, e.g. spermine, cationic linear or branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural or synthetic fusogenic lipids, natural or synthetic cationic lipids.

In an embodiment, the endosomolytic ligand may include a plurality of leucine or valine residues. The endosomolytic ligand may be polyleucine. In an embodiment, endosomolytic ligand may be Leu-Leu-Leu (LLL).

In an embodiment, the nanoimmunoconjugate may further comprise an imaging agent covalently linked with the polymalic acid-based molecular scaffold. As used herein, the term “imaging agent” refers to an element or functional group in a molecule that allows for the detection, imaging, and/or monitoring of the presence and/or progression of a condition(s), pathological disorder(s), and/or disease(s). The imaging agent may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

Suitable optical reporters may be, but are not limited to, fluorescent reporters or chemiluminescent groups. A wide variety of fluorescent reporter dyes, e.g., fluorophores, are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters may include xanthene dyes, such as fluorescein or rhodamine dyes. Fluorophores may be, but are not limited to, 1,5 IAEDANS; 1,8-ANS; 4-Methylumbelliferone; 5-carboxy-2,7-dichlorofluorescein; 5-Carboxy fluorescein (5-FAM); 5-Carboxynapthofluorescein (pH 10); 5-Carboxytetramethyl rhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethyl rhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; or YOYO-3. Many suitable forms of these fluorescent compounds are available and may be used.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566, contents of which are incorporated herein by reference as if fully set forth). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, mOrange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane. Suitable non-metallic isotopes include, but are not limited to, ¹¹C, ¹⁴C, ¹³N, ¹⁸F, ¹²³I, ¹²⁴I, ¹²⁵I, and ¹³¹I. Suitable radioisotopes include, but are not limited to, ⁹⁹mTc, ⁹⁵Tc, ¹¹¹In, ⁶²Cu, ⁶⁴Cu, Ga, ⁶⁸Ga, ⁴⁷Sc, ⁶⁴Cu, ⁶⁷CU, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In an embodiment, the imaging agent may comprise a chelating molecule. Suitable chelating agents include, but are not limited to, 1,4,7,10-tetraazocyclododecane-1,4,7,10-tetraacetic acid (DOTA); dibenzo-DOTA, diethylenetriaminepentaacetic acid (DTPA); 1,4,7,10-tetraazacyclododecane-1,4,7.10-tetrakis(2-propionic acid) (DOTMA); 1,4,8,11-tetrazacyclotetradecane-1,4,8,11-tetraacetic acid (TETA); 1,4,7,-tricarboxymethyl 1,4,7,10 teraazacyclododecane triacetic acid (DO3A); 1,4,7,10-tetraazacyclo-dodecan-1-(2-hydroxypropyl)-4,7,10-triacetic acid (HP-DO3A); ethylenediamine-tetraacetic acid (EDTA); bis-2 (hydroxybenzyl)-ethylene-diaminediacetic acid (HBED); 1,4,7-triazacyclo-nonane 1,4,7-triacetic acid (NOTA); BAD, EDTA, NTA, HDTA, their phosphonate analogs, and mixtures thereof. In an embodiment, the imaging agent may be Alexa Fluor 680™.

Without limitations, the nanoimmunoconjugate may be of any desired size. For example, the nanoimmunoconjugate may be of a size that allows the nanoimmunoconjugate to cross the blood brain barrier via transcytosis. In an embodiment, the nanoimmunoconjugate may range in size from about 1 nm to about 100 nm; from about 1 nm to about 10 nm; from about 10 nm to about 20 nm; from about 20 nm to about 30 nm; from about 30 nm to about 40 nm; from about 40 nm to about 50 nm; from about 50 nm to about 60 nm; from about 60 nm to about 70 nm; from about 70 nm to about 80 nm; from about 80 nm to about 90 nm; from about 90 nm to about 100 nm; from about 5 nm to about 90 nm; from about 10 nm to about 85 nm; from about 20 nm to about 80 nm; from about 25 nm to about 75 nm. In an embodiment, the nanoimmunoconjugate may be about 50 nm to about 70 nm in size. In an embodiment, the nanoimmunoconjugate may be 50 nm or less in size.

It will be understood by one of ordinary skill in the art that nanoimmunoconjugates may exhibit a distribution of sizes around the indicated “size.” Thus, unless otherwise stated, the term “size” as used herein refers to the mode of a size distribution of nanoimmunoconjugates, i.e., the value that occurs most frequently in the size distribution. Methods for measuring the size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

An embodiment provides a method for treating cancer. The method may comprise administering a therapeutically effective amount of a composition comprising any one of the nanoimmunoconjugates described herein to a subject in need thereof.

In an embodiment, the method for treating cancer may further comprise providing the composition comprising any one of the nanoimmunoconjugates described herein to a subject in need thereof.

In an embodiment, the method for treating cancer may comprise administering a therapeutically effective amount of any one of the nanoimmunoconjugates described herein to a subject in need thereof.

In an embodiment, the method for treating cancer may comprise co-administering a therapeutically effective amount of an anti-tumor immune response stimulator and a therapeutically effective amount of a nanoconjugate to a subject in need thereof, wherein the nanoconjugate comprises a polymalic acid-based molecular scaffold and at least one targeting ligand and at least one anti-cancer agent covalently conjugated or linked to the scaffold.

In an embodiment, the method may further comprise analyzing inhibition of tumor growth. The step of analyzing may include observing more than about 60%, 70%, 80% or about 90% inhibition of tumor growth in the subject. In an embodiment, the step of analyzing may include observing the inhibition of HER2/neu receptor signaling by suppression of Akt phosphorylation.

The phrase “therapeutically-effective amount” as used herein means that amount of a compound, material, or composition which is effective for producing some desired therapeutic effect in at least a sub-population of cells in an animal at a reasonable benefit/risk ratio applicable to any medical treatment. In connection with treating cancer, the “therapeutically effective amount” is that amount effective for preventing further development of a cancer or transformed growth, and even to effect regression of the cancer or solid tumor.

Determination of a therapeutically effective amount is generally well within the capability of those skilled in the art. Generally, a therapeutically effective amount can vary with the subject's history, age, condition, sex, as well as the severity and type of the medical condition in the subject, and administration of other agents alleviate the disease or disorder to be treated.

Toxicity and therapeutic efficacy may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. As used herein, the term ED denotes effective dose and is used in connection with animal models. The term EC denotes effective concentration and is used in connection with in vitro models.

The data obtained from the cell culture assays and animal studies may be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose may be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage may be monitored by a suitable bioassay.

The dosage may be determined by a physician and adjusted, as necessary, to suit observed effects of the treatment. Generally, the compositions may be administered so that the active agent is given at a dose from 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 g/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 tmg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

In an embodiment, the compositions may be administered at a dosage so that the active agent has an in vivo concentration of less than 500 nM, less than 400 nM, less than 300 nM, less than 250 nM, less than 200 nM, less than 150 nM, less than 100 nM, less than 50 nM, less than 25 nM, less than 20, nM, less than 10 nM, less than 5 nM, less than 1 nM, less than 0.5 nM, less than 0.1 nM, less than 0.05, less than 0.01, nM, less than 0.005 nM, less than 0.001 nM after 15 mins, 30 mins, 1 hr, 1.5 hrs, 2 hrs, 2.5 hrs, 3 hrs, 4 hrs, 5 hrs, 6 hrs, 7 hrs, 8 hrs, 9 hrs, 10 hrs, 11 hrs, 12 hrs or more of time of administration.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule may vary from once a week to daily depending on a number of clinical factors, such as the subject's sensitivity to the polypeptides. The desired dose may be administered every day or every third, fourth, fifth, or sixth day. The desired dose may be administered at one time or divided into subdoses, e.g., 2-4 subdoses and administered over a period of time, e.g., at appropriate intervals through the day or other appropriate schedule. Such sub-doses may be administered as unit dosage forms. In an embodiment, administration may be chronic, e.g., one or more doses daily over a period of weeks or months. Examples of dosing schedules may include administration daily, twice daily, three times daily or four or more times daily over a period of 1 week, 2 weeks, 3 weeks, 4 weeks, 1 month, 2 months, 3 months, 4 months, 5 months, or 6 months or more.

As used herein, the term “administer” refers to the placement of a composition into a subject by a method or route which results in at least partial localization of the composition at a desired site such that desired effect is produced. A compound or composition described herein may be administered by any appropriate route known in the art including, but not limited to, oral or parenteral routes, including intravenous, intramuscular, subcutaneous, transdermal, airway (aerosol), pulmonary, nasal, rectal, or topical (including buccal and sublingual) administration.

Exemplary modes of administration include, but are not limited to, injection, infusion, instillation, inhalation, or ingestion. “Injection” include, without limitation, intravenous, intramuscular, intraarterial, intrathecal, intraventricular, intracapsular, intraorbital, intracardiac, intradermal, intraperitoneal, trans tracheal, subcutaneous, subcuticular, intraarticular, sub capsular, subarachnoid, intraspinal, intracerebro spinal, and intrastemal injection and infusion. In an embodiment, the compositions may be administered by intravenous infusion or injection.

For administration to a subject, the nanoimmunoconjugate and/or the anti-tumor immune response stimulator may be provided in pharmaceutically acceptable compositions. Accordingly, an embodiment also provides pharmaceutical compositions comprising the nanoimmunoconjugate as disclosed herein. These pharmaceutically acceptable compositions may comprise a therapeutically-effective amount of one or more of the nanoimmunoconjugates, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, the nanoimmunoconjugate may be implanted into a patient or injected using a drug delivery system.

A variety of known controlled- or extended-release dosage forms, formulations, and devices may be adapted for use with the nanoimmunoconjugates and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, all of which are incorporated herein by reference as if fully set forth. These dosage forms may be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In an embodiment, the pharmaceutically acceptable composition may be formulated in dosage unit form for ease of administration and uniformity of dosage. The expression “dosage unit form” as used herein refers to a physically discrete unit of active agent appropriate for the subject to be treated.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zincstearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (S) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyllaurate; (13) agar; (14) buffering agents, such as magnesium hydroxide and aluminum hydroxide; (15) alginic acid; (16) pyrogen-free water; (17) isotonic saline; (IS) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants may also be present in the formulation. The terms such as “excipient”, “carrier”, “pharmaceutically acceptable carrier” or the likes are used interchangeably herein.

As used herein, the term “cancer” refers to an uncontrolled growth of cells that may interfere with the normal functioning of the bodily organs and systems. The cancer may be either a primary cancer, or a metastatic cancer, or both. Cancers that migrate from their original location and seed vital organs can eventually lead to the death of the subject through the functional deterioration of the affected organs. Metastasis is a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination.

As used herein, the term “cancer” also includes, but is not limited to, solid tumors and blood born tumors. The term cancer refers to disease of skin, tissues, organs, bone, cartilage, blood and vessels. The term “cancer” further encompasses primary and metastatic cancers. Examples of cancers that can be treated with the method of the invention include, but are not limited to solid tumors; brain cancer, including but not limited to gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas; breast cancer, including but not limited to ductal carcinoma in situ, invasive (or infiltrating) ductal carcinoma, invasive (or infiltrating) lobular carcinoma, adenoid cystic (or adenocystic) carcinoma, low-grade adenosquamous carcinoma, medullary carcinoma, mucinous (or colloid) carcinoma papillary carcinoma, tubular carcinoma, inflammatory breast cancer, Paget disease of the nipple, phyllodes tumor, triple negative breast cancer, metastatic breast cancer; carcinoma, including that of the bladder, breast, colon, kidney, lung, ovary, pancreas, stomach, cervix, thyroid, and skin, including squamous cell carcinoma; other tumors including melanoma, seminoma, tetratocarcinoma; tumors of the central and peripheral nervous system; and other tumors including, but not limited to, xenoderma, pigmentosum, keratoactanthoma, thyroid follicular cancer, and teratocarcinoma.

The methods disclosed herein are useful for treating patients who have been previously treated for cancer, as well as those who have not previously been treated for cancer. Indeed, the methods and compositions described herein may be used in first-line and second-line cancer treatments.

As used herein, the term “precancerous condition” has its ordinary meaning, i.e., an unregulated growth without metastasis, and includes various forms of hyperplasia and benign hypertrophy. Accordingly, a “precancerous condition” is a disease, syndrome, or finding that, if left untreated, can lead to cancer. It is a generalized state associated with a significantly increased risk of cancer. Premalignant lesion is a morphologically altered tissue in which cancer is more likely to occur than its apparently normal counterpart. Examples of pre-malignant conditions include, but are not limited to, oral leukoplakia, actinic keratosis (solar keratosis), Barrett's esophagus, atrophic gastritis, benign hyperplasia of the prostate, precancerous polyps of the colon or rectum, gastric epithelial dysplasia, adenomatous dysplasia, hereditary nonpolyposis colon cancer syndrome (HNPCC), Barrett's esophagus, bladder dysplasia, precancerous cervical conditions, and cervical dysplasia.

In an embodiment, the cancer may be selected from the group consisting of: breast cancer; ovarian cancer; brain cancer; gastrointestinal cancer; prostate cancer; carcinoma, lung carcinoma, hepatocellular carcinoma, testicular cancer; cervical cancer; endometrial cancer; bladder cancer; head and neck cancer; lung cancer; gastro-esophageal cancer, and gynecological cancer.

In an embodiment, the cancer may be breast cancer, including but not limited to ductal carcinoma in situ, invasive (or infiltrating) ductal carcinoma, invasive (or infiltrating) lobular carcinoma, adenoid cystic (or adenocystic) carcinoma, low-grade adenosquamous carcinoma, medullary carcinoma, mucinous (or colloid) carcinoma papillary carcinoma, tubular carcinoma, inflammatory breast cancer, Paget disease of the nipple, phyllodes tumor, triple negative breast cancer, metastatic breast cancer.

In an embodiment, the cancer may be a primary HER2+ breast cancer, triple negative breast cancer (TNBC) or their metastasis to the brain.

In an embodiment, the cancer may be brain cancer, including but not limited to gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas. In an embodiment, the brain cancer may be glioma, glioblastoma, or glioblastoma multiforme (GBM).

In an embodiment, the methods described herein may relate to treating a subject having or diagnosed as having cancer. Subjects having cancer may be identified by a physician using current methods of diagnosing cancer. Symptoms and/or complications of cancer which characterize these conditions and aid in diagnosis are well known in the art and may be, but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer, or exposure to risk factors for cancer (e.g. tobacco products, radiation, etc.) may also aid in determining if a subject is likely to have cancer or in making a diagnosis of cancer.

In an embodiment, the method may further comprise co-administering an additional therapeutic agent. The additional therapeutic agent may be selected from the group consisting of: an antibody, an enzyme inhibitor, an antibacterial agent, an antiviral agent, a steroid, a non-steroid-inflammatory agent, an antimetabolite, a cytokine, a cytokine blocking agent, an adhesion molecule blocking agent, and a soluble cytokine receptor.

In an embodiment, the method may further comprise co-administering one or more additional anti-cancer therapy to the patient. In an embodiment, the additional therapy may be selected from the group consisting of surgery, chemotherapy, radiation therapy, thermotherapy, immunotherapy, hormone therapy, laser therapy, anti-angiogenic therapy, and any combinations thereof. In an embodiment, the additional therapy may comprise administering an anti-cancer agent to the patient.

In an embodiment, the method may comprise co-administering the nanoimmunoconjugate and an anti-cancer agent or chemotherapeutic agent to the subject.

In an embodiment, the method may comprise co-administering an antineoplastic agent. The antineoplastic agents may include agents for overcoming trastuzumab resistance. A variety of agents including monoclonal antibodies, recombinant proteins, and drugs, are known to have activity in treating breast cancer, and are here contemplated to be useful agents in combination with compositions described herein.

In an embodiment, the method may include co-administering paclitaxel (taxol, Bristol-Myers Squibb); docetaxel (taxotere, Sanofi-Aventis); dasatinib, (Sprycel®, Bristol-Myers Squibb) a small-molecule tyrosine kinase inhibitor; gefitinib (Iressa, Astra Zeneca and Teva), an EGFR inhibitor; trastuzumab; an agent that decreases levels of phosphorylated HER2 and phosphorylated HEM; an agent that induces caspase-independent apoptosis as determined by the lack of an effect of caspase inhibitors on apoptosis; an agent that affects DNA repair machinery and leads to accumulation of double-stranded breaks (DSBs); erlotinib (Tarceva, Roche), an inhibitor of EGFR; an agent that affects a transcription factor associated with Williams-Beuren syndrome (WSTF, also known as BAZ 1B), a tyrosine kinase component of the WICH complex (WSTF-ISWI ATP-dependent chromatin-remodeling complex), that regulates the DNA damage response through phosphorylation of Tyr142 of H2AX; lapatinib (Tyverb®, GSK), a dual EGFR/HER2 tyrosine kinase inhibitor; pertuzumab (2c4, omnitarg, Genentech), a monoclonal antibody specific for the extracellular domain of HER2 protein; trastuzumab-DM1 comprised of trastuzumab and DM1, an agent that is an inhibitor of tubulin polymerization derived from maytansine; a PI3K pathway inhibitor; HER2 vaccines and adoptive immunotherapy targeting the HER2 extracellular domain; ertumaxomab (Rexomum, Fresenius Biotech GmbH), a bispecific antibody targeting HER2 and CD3 on T cells; defucosylated trastuzumab; or any combinations thereof.

The following list includes particular embodiments of the present invention. But the list is not limiting and does not exclude alternate embodiments, or embodiments otherwise described herein. Percent identity described in the following embodiments list refers to the identity of the recited sequence along the entire length of the reference sequence.

### EMBODIMENTS

1. A nanoimmunoconjugate comprising a polymalic acid-based molecular scaffold, at least one targeting ligand, at least one anti-tumor immune response stimulator and at least one anti-cancer agent, wherein the targeting ligand, the anti-tumor immune response stimulator and the anti-cancer agent are covalently linked to the polymalic acid-based molecular scaffold.
2. The nanoimmunoconjugate of embodiment 1, wherein the anti-tumor immune response stimulator is selected from the group consisting of: an antisense oligonucleotide (AON), an siRNA oligonucleotide, an antibody, a polypeptide, an oligopeptide and a low molecular weight drug.
3. The nanoimmunoconjugate of one or both embodiments 1 and 2, wherein the anti-tumor immune response stimulator is an antibody.
4. The nanoimmunoconjugate of any one or more of embodiments 1-3, wherein the anti-tumor immune response stimulator is selected from the group consisting of: an antibody against PD-1, an antibody against PD-L1, an antibody against PD-L2, an antibody against CTLA-4, or a combination thereof.
5. The nanoimmunoconjugate of embodiment 2, wherein the anti-tumor immune response stimulator is an antisense oligonucleotide or an siRNA comprising a sequence complementary to a sequence contained in an mRNA transcript of an immune checkpoint protein.
6. The nanoimmunoconjugate of embodiment 5, wherein the antisense oligonucleotide is a morpholino antisense oligonucleotide.
7. The nanoimmunoconjugate of embodiment 6, wherein the antisense oligonucleotide comprises a sequence with at least 90% identity to a sequence selected from the group consisting of SEQ ID NOS: 4-7.
8. The nanoimmunoconjugate of embodiment 1, wherein the anti-tumor immune response stimulator is an inhibitor of an immune checkpoint protein.
9. The nanoimmunoconjugate of any one or more of embodiments 1 and 8, wherein the anti-tumor immune response stimulator is an immunostimulatory cytokine.
10. The nanoimmunoconjugate of embodiment 9, wherein the cytokine is IL-2 or IL-12.
11. The nanoimmunoconjugate of any one or more of embodiments 1-10, wherein the anti-cancer agent is selected from the group consisting of: an antisense oligonucleotide, an siRNA oligonucleotide, an antibody, a polypeptide, an oligopeptide and a low molecular weight drug.
12. The nanoimmunoconjugate of any one or more of embodiments 1-11, wherein the anti-cancer agent is the antisense oligonucleotide comprising a sequence with at least 90% identity to a sequence selected from the group consisting of SEQ ID NO: 1, 2 and 8.
13. The nanoimmunoconjugate of any one or more of embodiments 1-11, wherein the anti-cancer agent is an antisense oligonucleotide or an siRNA comprising a sequence complementary to a sequence contained in an mRNA transcript of a human epidermal growth factor receptor (HER), or the serine-threonine protein kinase (CK2).
14. The nanoimmunoconjugate of any one or more of embodiments 1-11 and 13, wherein the anti-cancer agent is an antisense oligonucleotide comprising a sequence complementary to a sequence with at least 90% identity to the sequence of SEQ ID NO: 3.
15. The nanoimmunoconjugate of any one or more of embodiments 1-11, wherein the anti-cancer agent is an anti-HER2/neu antibody.
16. The nanoimmunoconjugate of any one or more of embodiments 1-11 and 15, wherein the anti-HER2/neu antibody is Herceptin®.
17. The nanoimmunoconjugate of any one or more of embodiments 1-16, wherein the nanoimmunoconjugate comprises at least two different anti-cancer agents covalently linked to the polymalic acid-based molecular scaffold
18. The nanoimmunoconjugate of any one or more of embodiments 1-17, wherein the targeting ligand binds specifically to a vasculature protein in a tumorigenic cell or cancer cell.
19. The nanoimmunoconjugate of any one or more of embodiments 1-18, wherein the vasculature protein comprises a transferrin receptor protein.
20. The nanoimmunoconjugate of any one or more of embodiments 1-19, wherein the targeting ligand is an antibody.
21. The nanoimmunoconjugate of any one or more of embodiments 1-20, wherein the nanoimmunoconjugate further comprises a PK modulating ligand covalently linked with the polymalic acid-based molecular scaffold.
22. The nanoimmunoconjugate of embodiment 21, wherein the PK modulating ligand is polyethylene glycol (PEG).
23. The nanoimmunoconjugate of any one or more of embodiments 1-22, wherein the nanoimmunoconjugate further comprises an endosomolytic ligand covalently linked with the polymalic acid-based molecular scaffold.
24. The nanoimmunoconjugate of embodiment 23, wherein the endosomolytic ligand comprises a plurality of leucine or valine residues.
25. The nanoimmunoconjugate of embodiment 24, wherein the endosomolytic ligand is Leu-Leu-Leu (LLL).
26. The nanoimmunoconjugate of any one or more of embodiments 1-25, wherein the nanoimmunoconjugate further comprises an imaging agent covalently linked with the polymalic acid-based molecular scaffold.
27. A pharmaceutically acceptable composition comprising an nanoimmunoconjugate of any one or more of embodiments 1-26 and a pharmaceutically acceptable carrier or excipient.
28. A method for treating cancer in a subject comprising: providing a nanoimmunoconjugate of any one or more of embodiments 1-26 and administering a therapeutically effective amount of a nanoimmunoconjugate to the subject.
29. The method of embodiment 28, wherein the step of administering results in treating, reducing the severity or slowing the progression of cancer in the subject.
30. The method of one or both embodiments 28 and 29, wherein the cancer is a primary cancer, a metastatic cancer, or both.
31. The method of any one or more of embodiments 28-30, wherein the cancer is a primary HER2+ breast cancer, triple negative breast cancer (TNBC) or their metastasis to the brain.
32. The method of any one or more of embodiments 28-30, wherein the cancer is glioma or glioblastoma.
33. A method for treating cancer in a subject, comprising: providing a nanoconjugate comprising a polymalic acid-based molecular scaffold and at least one targeting ligand and at least one anti-cancer agent covalently linked to the scaffold; and co-administering a therapeutically effective amount of an anti-tumor immune response stimulator and a therapeutically effective amount of the nanoconjugate to a subject.
34. The method of embodiment 33, wherein the anti-tumor immune response stimulator is selected from the group consisting of: an antisense oligonucleotide (AON), an siRNA oligonucleotide, an antibody, a polypeptide, an oligopeptide and a low molecular weight drug.
35. The method of one or both embodiments 33 and 34, wherein the anti-tumor immune response stimulator is an antibody, wherein the antibody is selected from the group consisting of: an antibody against PD-1 antibody, an antibody against PD-L1, an antibody against PD-L2, an antibody against CTLA-4, or a combination thereof.
36. The method of one or both embodiments 33 and 34, wherein the anti-tumor immune response stimulator is an antisense oligonucleotide or an siRNA comprising a sequence complementary to a sequence contained in an mRNA transcript of an immune checkpoint protein.
37. The method of one or both embodiments 33 and 34, wherein the anti-tumor immune response stimulator is an antisense oligonucleotide and comprises a sequence with at least 90% identity to a sequence selected from the group consisting of SEQ ID NOS: 4-7.
38. The method of one or both embodiments 33 and 34, wherein the anti-tumor immune response stimulator is an inhibitor of an immune checkpoint protein.
39. The method of any one or more of embodiments 33, 34 and 38, wherein the anti-tumor immune response stimulator is an immunostimulatory cytokine, and the cytokine is selected from IL-2 or IL-12.
40. The method of any one or more of embodiments 33-39, wherein the anti-cancer agent is selected from the group consisting of: an antisense oligonucleotide, an siRNA oligonucleotide, an antibody, a polypeptide, an oligopeptide and a low molecular weight drug.
41. The method of any one or more of embodiments 33-40, wherein the anti-cancer agent is the antisense oligonucleotide and comprises a sequence with at least 90% identity to a sequence selected from the group consisting of SEQ ID NO: 1, 2 and 8.
42. The method of any one or more of embodiments 33-40, wherein the anti-cancer agent is an antisense oligonucleotide or an siRNA comprising a sequence complementary to a sequence contained in an mRNA transcript of a human epidermal growth factor receptor (HER), or the serine-threonine protein kinase (CK2).
43. The method of any one or more of embodiments 33-40 and 42, wherein the anti-cancer agent is an antisense oligonucleotide and comprises a sequence complementary to a sequence with at least 90% identity to the sequence of SEQ ID NO: 3.
44. The method of any one or more of embodiments 33-40, wherein the anti-cancer agent is an anti-HER2/neu antibody.
45. The method of any one or more of embodiments 33-40 and 44, wherein the anti-cancer agent is Herceptin®.
46. The method of any one or more of embodiments 33-45, wherein the targeting ligand binds specifically to a vasculature protein in a tumorigenic cell or cancer cell.
47. The method of any one or more of embodiments 33-46, wherein the vasculature protein comprises a transferrin receptor protein.
48. The method of any one or more of embodiments 33-47, wherein the targeting ligand is an antibody.
49. The method of any one or more of embodiments 33-48, wherein the nanoconjugate further comprises a PK modulating ligand covalently linked with the polymalic acid-based molecular scaffold.
50. The method of embodiment 49, wherein the PK modulating ligand is polyethylene glycol (PEG).
51. The method of any one or more of embodiments 33-50, wherein the nanoconjugate further comprises an endosomolytic ligand covalently linked with the polymalic acid-based molecular scaffold.
52. The method of any one or more of embodiments 33-51, wherein the endosomolytic ligand comprises a plurality of leucine or valine residues.
53. The method of any one or more of embodiments 33-51, wherein the endosomolytic ligand is Leu-Leu-Leu (LLL).
54. The method of any one or more of embodiments 33-53, wherein the nanoimmunoconjugate further comprises an imaging agent covalently linked with the polymalic acid-based molecular scaffold.
55. The method of any one or more of embodiments 33-54, wherein the cancer is a primary cancer, a metastatic cancer, or both.
56. The method of any one or more of embodiments 33-55, wherein the cancer is a primary HER2+ breast cancer, triple negative breast cancer (TNBC) or their metastasis to the brain.
57. The method of any one or more of embodiments 33-55, wherein the cancer is glioma or glioblastoma.
58. The method of any one or more of embodiments 33-57, wherein the method further comprises co-administering an additional therapeutic agent to the subject.
59. The method of any one or more of embodiments 33-58, wherein the method further comprises co-administering one or more additional anti-cancer therapy to the subject.
60. The method of embodiment 59, wherein the additional anti-cancer therapy is selected from the group consisting of surgery, chemotherapy, radiation therapy, thermotherapy, immunotherapy, hormone therapy, laser therapy, anti-angiogenic therapy, and any combinations thereof.
61. The method of any one or more of embodiments 33-60, wherein the subject is a mammal.
62. The method of embodiment 61, wherein the mammal is selected from the group consisting of: a rodent, an experimental human-breast tumor-bearing nude mouse and a human.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

### Example 1—Breast Cancer Treatment Design

Success with CTLA-4 and PD-1 blockade in treating multiple cancers highlights that it is ever more important understanding the complexity of the immune and inflammatory systems in the development and progression of breast cancer. The microenvironmental immune system of breast cancer is dysregulated. Normal immune system is balanced with both stimulatory and inhibitory components. Cancer cells acquire the capability to evade immune surveillance by utilizing the mechanism of peripheral tolerance and by inactivating cytotoxic T lymphocytes (CTL). The following are two approaches that are of special interest.

Blockade of CTL-associated antigen 4 (CTLA-4) using antagonistic mAb ipilimumab (Yervoy®) was the first strategy to achieve a significant clinical benefit for late stage (stage IV) melanoma patients in two Phase III clinical trials, fueling the notion of immunotherapy being the breakthrough strategy for oncology in 2013. Humanized mAbs against immune system response modulators (“checkpoint inhibitors” such as CTLA-4 mAb ipilimumab, and PD-1 mAb pembrolizumab (Keytruda®) received FDA approval for melanoma therapy. Their effect is related to suppression of Treg (CD4+CD25+FoxP3+) that attenuate immune response by CTL. Although systemic CTLA-4 or programmed cell death-1 (PD-1) mAbs contribute to suppression of some tumors, they have low efficacy against brain and breast tumors and require co-treatment with radiation for the effect to appear.

Currently, it is known that both CD28 and CTLA-4 modulate T-cell activation by providing second signals that either induce or repress immune responses. CD28 is a co-stimulator of the T-cell receptor/major histocompatibility complex (TCR/MHC) interaction and leads to induction of an immune response. CTLA-4 is a co-inhibitor that leads to immunosuppression. The interaction of CTLA-4 with its ligands are of higher avidity than that of CD28, therefore, CTLA-4 out-competes the stimulatory signal resulting in decreased T-cell proliferation and IL-2 production, and ultimately suppression of the T-cell immune response including CTL activity against cancer cells. Thus, blockage of ligand binding to CTLA-4 through the use of antagonistic Abs, favors the interaction of CD28 with the ligands leading to immune activation.

PD-1(CD279) is a type I transmembrane receptor member of the immunoglobulin superfamily, expressed by activated T cells, and binds to two ligands, PD-L1 (B7-H1, CD274) and PD-L2 (B7-DC, CD273), both of which are part of the B7 immunoglobulin superfamily. Given the selective immune suppressive signals delivered by cancer, it was predicted that the blockade of PD-1/PD-L1 pathway will have greater antitumor activity and fewer side effects compared to CTLA-4 blockade. Anti-CTLA-4/PD-1 mAbs turn off the inhibitory mechanism to allow CTL to eliminate cancer cells, but the exact mechanism of anti-tumor activity of anti-CTLA-4 mAbs remains controversial and a second mechanism for CTLA-4/PD-1 mAbs has been proposed.

Subsequently, many trials have tested various immune checkpoint modulators in malignancies. Specifically in breast cancer, the elevated numbers of Tregs and decreased ratios of CD8 T cells/Treg are correlated with a poor prognosis. Higher expression of CTLA-4 at both protein and mRNA level was found in all specimens of breast tumor patients and higher mRNA level of CTLA-4 is correlated with the obvious axillary lymph node metastases and higher clinical stage. Thus, CTLA-4 blockade is a meaningful way for breast cancer therapy.

However, there are several drawbacks with CTLA-4 Ab monotherapy for treatment of cancer including significant toxicities ensuing after the suppression of Tregs as a result of autoimmune effect and lack of tumor-specific immunity, which greatly limit the application of mAb therapeutics.

As described herein, for the breast cancer treatment immunomodulators, check point inhibitors CTLA-4 and/or PD-1 and/or cytokines IL-2 and/or IL-12 were used and in combination with suppression of protein kinase CK2 and EGFR/EGFRvIII using AONs.

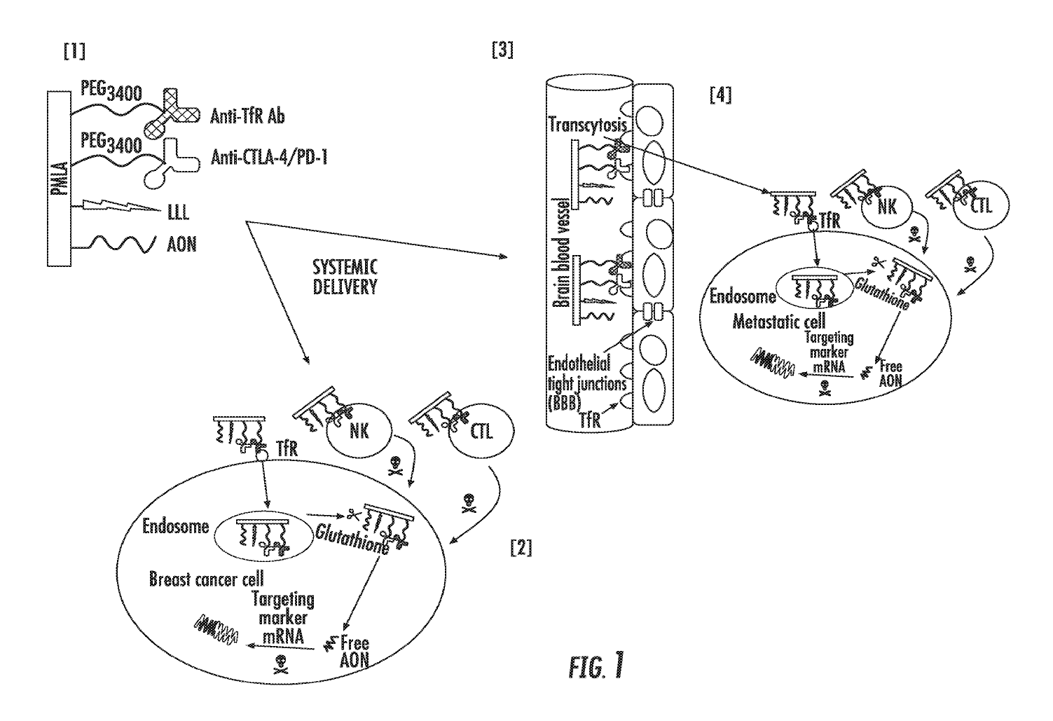

The combined power of cytotoxic AON, and checkpoint inhibitors was harnessed as part of nanoimmunoconjugates (NIC), capable of targeting breast cancer, including its most devastating stage, which is brain metastasis. This approach was designed to directly eliminate cancer cells and also elicit a local and systemic long-term broad-spectrum immune response. FIG. 1 illustrates an exemplary structure and mechanism of action of nanoimmunoconjugates (NIC) in the context of breast cancer. Referring to FIG. 1, [1] indicates a general structure of NIC that can be used alone or in combination. It has a PMLA backbone, mPEG 5000 for stability, an endosomal escape unit (LLL), an anti-TfR mAb for BBB and breast tumor targeting, and an AON against CK2 to induce tumor cytotoxicity. The latter can be replaced by or combined with AON against HER2/neu. NIC also contains the checkpoint inhibitor mAb anti-PD-1 or -CTLA-4, which can be replaced by anti-HER2/neu Ab and IL-2 for tumor targeting and immunostimulation. Free cytokines can also be directly conjugated to NIC. Preference is given to IL-2, as IL-12 is well induced by checkpoint inhibitors. Proteins (mAbs and derivatives) are bound to PMLA through a PEG₃₄₀₀ linker. In FIG. 1, [2-4] refers to the proposed mechanisms of action. After systemic intravenous (i.v.) administration, [2] NIC reaches the tumor through TfR or HER2/neu tumor associated antigens (Ags) and is delivered into the cells by receptor-mediated endocytosis. LLL allows endosomal escape, and AON (e.g., to CK2) is cleaved off by cytoplasmic glutathione to block target mRNA. The attached mAbs to checkpoint proteins interact with Treg in the tumor and in circulation eliciting CTL response. In case of breast cancer brain metastases, blood-brain barrier (BBB) is crossed by TfR-mediated transcytosis [3]. Once in the brain, the NIC cytokine on AbFP are capable of directly activating NK and CTL cells and linking innate with adaptive immunity, while the checkpoint inhibitor of NIC removes the Treg “brake” from CTL maximizing their tumor killing potential [4].

Referring to FIG. 1, PMLA is used as a scaffold for conjugation with anti-tumor immune response stimulators, anti-cancer drugs, and tumor targeting Abs. As potent immunostimulators, (1) an antagonistic Ab targeting PD-1 or CTLA-4 (checkpoint inhibitor mAbs) are used to turn off the inhibitory mechanism and allow CTL to eliminate cancer cells; and (2) AbFP composed of a mAb specific for HER2/neu genetically fused to the potent immunostimulatory cytokines IL-2 and/or IL-12 that would activate both NK and CTL cells, and boost the activity of checkpoint inhibitor mAbs. As cytotoxic anti-cancer drugs, AON are used to suppress the expression of HER2/neu and/or the master signaling regulator CK2, important survival effectors of breast cancer cells. This cytotoxic effect would increase apoptosis of the tumor cells facilitating their phagocytosis by Ag presenting cells (APC) such as dendritic cells (DC), leading to subsequent increase in adaptive CTL anti-tumor immune response. This effect would lead to antigen spreading, with an adaptive immune response against tumor Ags, minimizing the changes of tumor escape and relapse and increasing long-term broad-spectrum immunity. The combination of powerful effectors maximizes anti-tumor activity (synergistic effect).

The advantages of this approach are as follows. NIC allows the simultaneous delivery of nanodrugs to breast cancer tumors throughout a number of biological membranes including the endothelial system, cancer cell and endosomal membranes, as well as BBB to treat breast cancer metastases in the brain. NIC bears CTL-activating mAbs (PD-1 or CTLA-4) to boost systemic and local anti-tumor responses. Blocking checkpoint proteins CTLA-4 and PD-1 through gene knockdown using anti CTLA-4 and PD-1 AON allows to reduce the known toxicity of therapeutic Abs. Inclusion of Ab fused to cytokines (IL-2 or IL-12), together with the checkpoint inhibition strategies, is designed to orchestrate a strong innate and adaptive immune response. Combination of immune system activation with anti-cancer cytotoxic drugs by inhibiting protein kinase CK2 or HER2/neu, is designed to eradicate breast tumor mass directly and through the immune cytotoxic response. The process described herein is the combination of two approaches: the use of a maximum loaded NIC as single drug as well as the co-administration of a NIC partially loaded with some of the moieties plus free immunostimulators, in order to select the most efficacious treatment regimen. Overall, a NIC is designed to provide simultaneous specific cancer cell killing (internal attack) and stimulation of anti-tumor immune response (external attack), which significantly increases anti-tumor efficacy.

### Example 2—Nanoimmunoconjugates Efficiently Treat Breast Cancer in Animal Models

An anti-tumor study in immunocompetent mice bearing a syngeneic mammary tumor D2F2/E2 expressing human HER2/neu showed that only the NIC significantly improved survival and was well tolerated. This effect was observed despite the fact that only two low doses were used. Importantly, NIC significantly increased serum levels of murine anti-HER2/neu IgG1 and IgG2a, which in mice are linked to humoral (T_{H}2) and cell-mediated (T_{H}1) immune responses, consistent with the observed anti-tumor protection. The superior NIC anti-tumor activity can be explained by the activation of immune effector cells in immunocompetent mice (NK and CTL cells) and also by AON cytotoxicity. Tumor targeting occurred through HER2/neu binding and partially, through the enhanced permeability and retention (EPR) effect. However, this NIC did not have anti-mouse TfR mAb that would enhance murine tumor targeting and hence, anti-tumor activity.

A NIC with PMLA containing LLL, mPEG, IL-2, and the anti-mouse TfR mAb was developed. All conjugated components were fully bioactive.

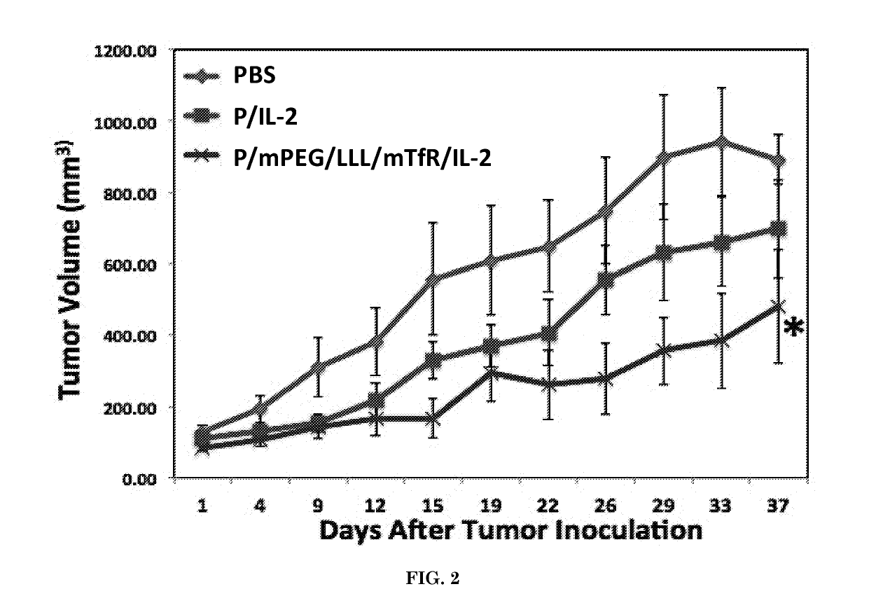

FIG. 2 is a set of line graphs illustrating anti-tumor activity of NIC (P/mPEG/LLL/mTfR/IL-2; x-mark) in a human xenograft breast cancer (BT-474) model compared to control treatments with PBS (closed diamond) and P/IL-2 (closed square). FIG. 2 illustrates anti-tumor activity of NIC in a human xenograft breast cancer (BT-474) model. Referring to FIG. 2, 10⁷ BT-474 cells were injected subcutaneously (s.c.) (right flank) on day 0 in female nude mice. Therapy began when tumors reached an average of 160 mm³ (day 20). Mice were divided into 3 groups (n=6 each), and given PBS, P/IL-2, or NIC i.v. twice a week. The dose for P/IL-2 is 2 mg/kg (˜50 μg, free or bound to the NIC) and treatment was performed 8 times. Average tumor volumes are significantly lower only in NIC group compared to PBS at day 37 when mice were euthanized (* p<0.01, two-way ANOVA, shown with SD). Referring to FIG. 2, the NIC showed significantly improved survival in nude mice bearing BT-474 human breast cancer and was well tolerated.

Nanoimmunodrugs containing anti-CTLA-4 mAb: a version of PMLA containing LLL, mPEG, anti-mouse TfR mAb, and an anti-mouse CTLA-4 mAb (BioXcell) was developed. The antibodies may be obtained also be from other companies. The activity of this NIC was tested BALB/c mice bearing s.c. syngeneic murine mammary carcinoma cells D2F2. This cell line was the parental of the D2F2/E2 not expressing human HER2/neu, described above.

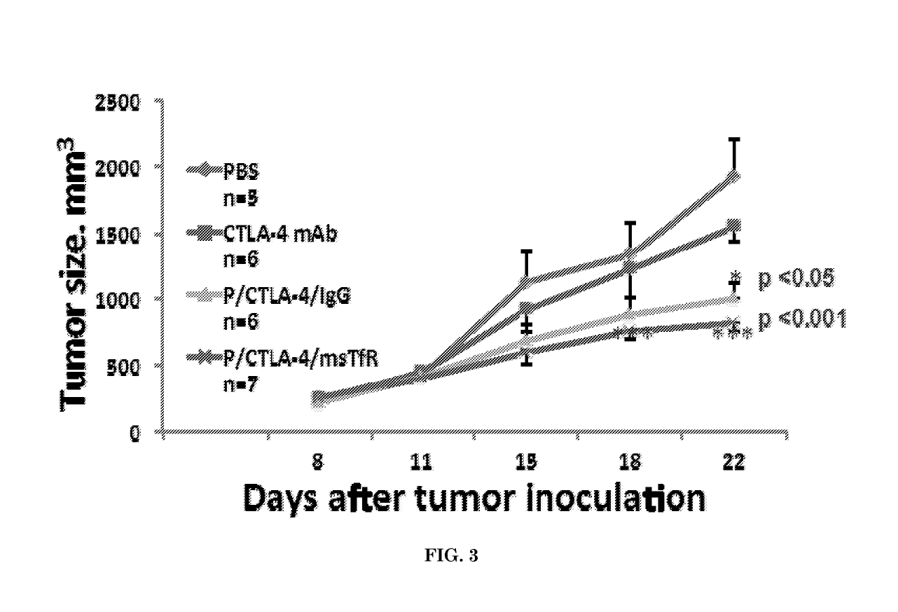

FIG. 3 illustrates anti-tumor activity of NIC in BALB/c mice bearing s.c. D2F2 syngeneic mammary tumors. Referring to FIG. 3, 10⁶ D2F2 cells were injected s.c. (right flank) on day 0. Therapy began when tumors reached an average size of ˜160 mm³ (Day 8). Mice were treated i.v. with PBS (n=5), 5 mg/kg of free anti-CTLA-4 mAb (n=6), NIC conjugated with anti-CTLA-4 Ab and an IgG negative control mAb (n=6) or NIC conjugated to anti-CTLA-4 and anti-TfR mAbs (n=7), on days 8, 11, 15, 18, and 22. On day 23 mice were euthanized and sera collected. Average tumor volumes are indicated with SD. *p<0.05, ** p<0.001 (two-way ANOVA). Referring to FIG. 3, tumor growth was significantly inhibited in animals treated with the NIC containing the anti-CTLA-4 Ab compared to free anti-CTLA-4 Ab, which can be explained by superior tumor targeting due to the EPR effect and targeting of tumor infiltrating T cells. However, tumor growth was inhibited to a greater extent in mice treated with NIC containing both anti-CTLA-4 and anti-TfR Abs.

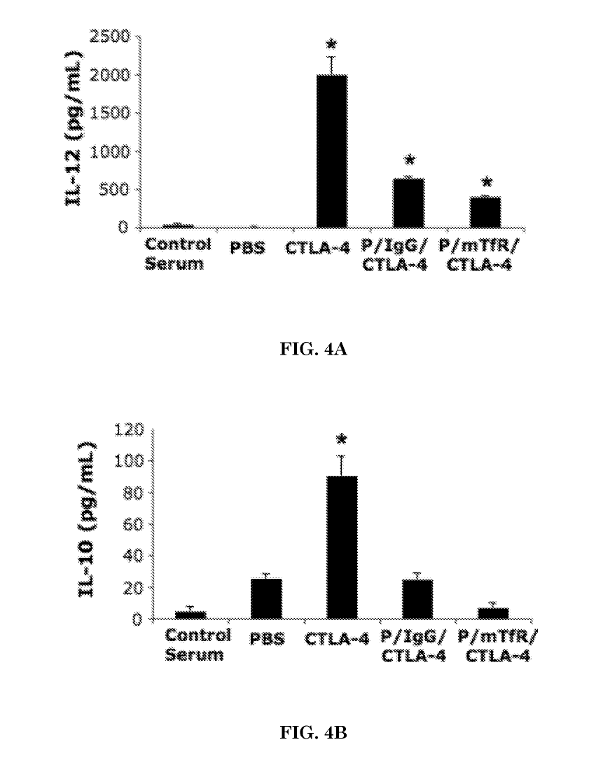

FIGS. 4A-4B illustrate preferential IL-12 (FIG. 4A) and IL-10 (FIG. 4B) activation induced by anti-CTLA-4 in BALB/c mice with s.c. D2F2 syngeneic mammary tumors. Referring to these figures, pooled serum samples from 4 randomly selected animals in each i.v. treated group were diluted 1:2, and tested in the Magnetic Luminex Screening Assay. Data for murine IL-12 and IL-10 are averages of 2 independent experiments in duplicate. Error bars indicate SEM. *p<0.05 vs. control sera from naïve mice and mice treated with PBS (Student's t-test). Referring to FIG. 4A, all treatments with anti-CTLA-4 mAb elicited significant IL-12 increase. Referring to FIG. 4B, IL-10 increase was seen only with free anti-CTLA-4 mAb. It was observed that IL-12 response is over 20-fold higher than IL-10. Referring to FIG. 4A, increased levels of serum murine IL-12 were observed in both NIC groups, consistent with preferential induction of a T_{H}1 cell-mediated immune response. These data suggest that the anti-CTLA-4 mAb bound to the PMLA was active and the anti-cancer activity was mediated by CTL. High serum IL-12 and IL-10 (although much less than IL-12) levels were observed in animals treated with the anti-CTLA-4 mAb alone. IL-10 is produced by T_{H}2 clones. Thus, these data are consistent with the induction of both a T_{H}1 cell-mediated and T_{H}2 humoral immune response. The presence of anti-CTLA-4 mAb on NIC resulted in a small but significant increase in both IL-12 and IL-10 levels. The fact that, in contrast to NIC, anti-cancer activity was not observed in mice treated with free anti-CTLA-4 mAb can be explained by NIC's effective induction of local CTL response in the tumor microenvironment as a result of tumor targeting. In addition, NIC and free anti-CTLA-4 Ab have different pharmacokinetics, and the serum samples were taken 1 day after the last treatment. Thus, higher levels of IL-12 and IL-10 (early activation markers) may have been detected at earlier time points in the NIC groups. In fact, measuring murine Abs (late activation markers) against D2F2 cells, the IgGa levels (associated with T_{H}2 response) were found to be consistent with the level of anti-tumor activity in different groups, being the highest in case of NIC conjugated with anti-CTLA-4 and anti-mTfR mAbs.

Using the above strategy, an initial study growing D2F2 cells intracranially was conducted to mimic breast cancer brain metastasis. This study was terminated at day 12 and mice received 2 treatments instead of 5. Mice had to be euthanized at day 12 because with the initial high dose of these aggressive cells in the brain (10⁵) they were exhibiting neurological symptoms.

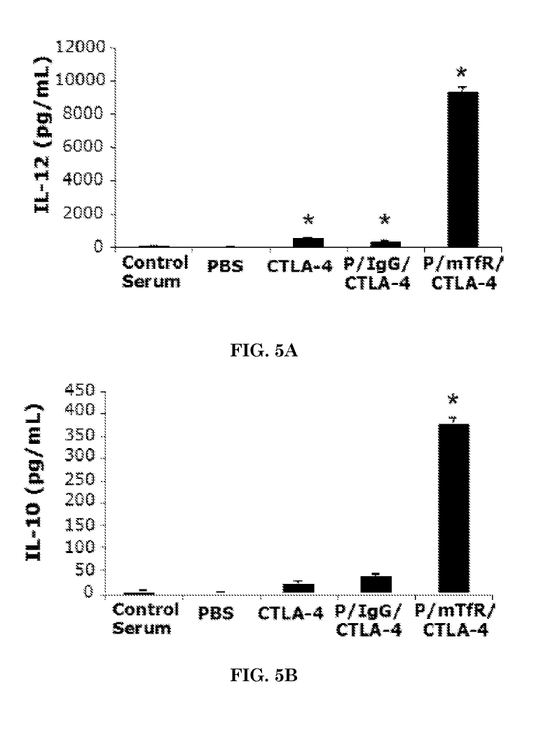

FIGS. 5A-5B illustrate immunostimulation in animals with intracranial D2F2 tumors (brain metastatic model). Mice (n=4 per group) were inoculated i.c. with 105 D2F2 cells (Day 0) and treated i.v. on days 5 and 8 with PBS or 5 mg/kg of free anti-CTLA-4 Ab or the same Ab conjugated with either control IgG Ab or mTfR Ab. On day 12 mice were euthanized, sera collected and pooled, and tested for murine IL-12 (FIG. 5A) and IL-10 (FIG. 5B) levels. Referring to these figures, it was observed that only tumor-targeted NIC passing BBB triggered high cytokine response. IL-12 response was over 20-fold higher than IL-10. It was observed that at this early time, high IL-12 levels were found in mice treated with NCI loaded with anti-CTLA-4 and anti-mTfR mAbs, consistent with superior tumor targeting and CTL activation. Referring to FIG. 5B, this group also showed early IL-10 signal, although at much lower levels compared to IL-12 (e.g., the difference in the figure's Y axis scales). Based on this initial experience a second preliminary study was performed, initially inoculating the animals with less tumor cells (10⁴ instead of 105) allowing more therapeutic administrations and longer survival.

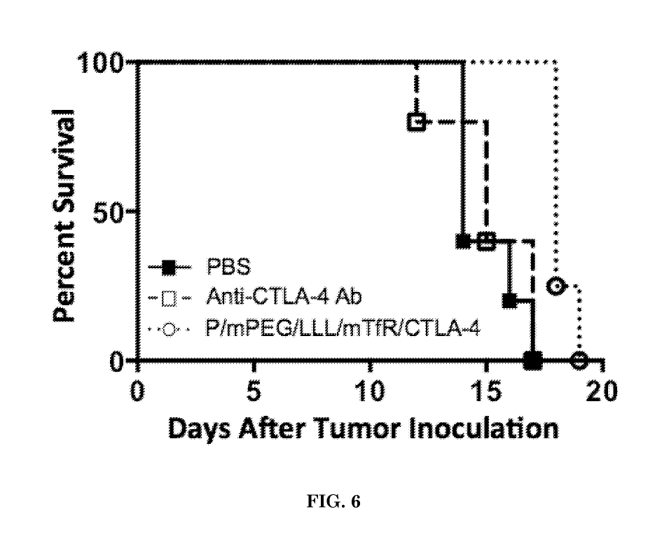

FIG. 6 illustrates Kaplan-Meier survival curves for BALB/c mice bearing intracranial mammary D2F2 tumors (brain metastatic model). 10⁴ D2F2 cells were injected intracranially on day 0. Systemic therapy was on days 3, 7, 10, 14 because D2F2 cells are very aggressive for brain tumors survival. Mice were treated with PBS, (n=5), 5 mg/kg or free anti-CTLA-4 Ab (n=6) or NIC conjugated to anti-CTLA-4 Ab and anti-TfR Ab (n=7). Survival in PBS and free CTLA-4 Ab groups were similar, but survival with NIC bearing anti-CTLA-4 and anti-TfR Abs was significantly longer (p<0.006, log-rank test). Tumor local delivery of CTLA-4 Ab as part of NIC is critical for immune system response to brain tumors.

Referring to FIG. 6, significant survival of mice with this particularly aggressive tumor was observed following treatment with the NIC conjugated to anti-CTLA-4 Ab and anti-TfR mAb.

All the data described herein were obtained with the NIC partially (single anti-cancer component) loaded and used alone. A much stronger anti-tumor activity is observed with a fully loaded NIC as well as NIC partially loaded and co-administered with free immunostimulators.

### Example 3—Synthesis and In Vitro Characterization of the Nanoimmunoconjugates for Treatment of Breast Cancer

NIC versions containing multi-pronged anti-cancer functions with the capacity of targeting breast cancer were developed. NIC containing multiple functional groups were synthesized in a controlled way with high reproducibility. The designed NICs are designed to deliver two different kinds of anti-cancer agents: immunostimulators (AbFP and/or checkpoint inhibitor mAb) and cytotoxic AON. The AON need to enter the cancer cell cytoplasm to function through endosome escape mechanism. In a thorough study, the effective endosome membranolysis by PMLA copolymer was confirmed when using pH-sensitive LLL. The P/LLL was found to permeate biological membrane through a “barrel-stave” mechanism, which allows more efficient endosomal release into the cytoplasm.

Methodology

Production of PMLA:

Because NICs contain multiple components, the success of the synthesis and its reproducibility was monitored. A synthesis with controlled conjugation of each component was developed. Each step of conjugation was verified with SEC-HPLC. Production and purification of PMLA from the slime mold Physarum polycephalum for NIC synthesis was performed as described. The PMLA was highly purified and characterized for reproducible synthesis of NIC. PMLA-based nanodrug synthesis is well established and is highly reproducible. The NIC variants can be synthesized similar to that described herein. PMLA of m.w. (weight-averaged molecular weight)=50,000 Da (polydispersity P=1.1) was prepared by fractionation on Sephadex G25 fine.

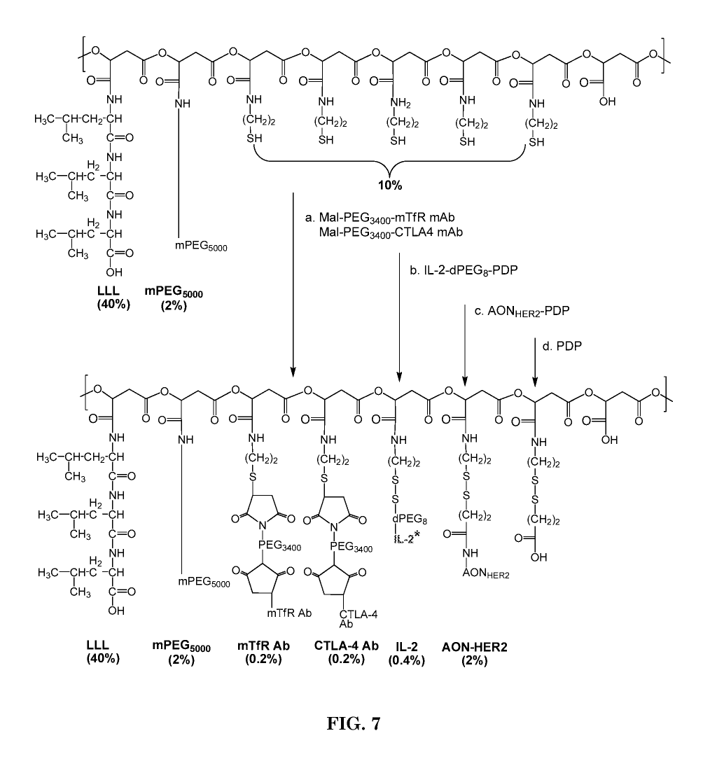

FIG. 7 illustrates the synthesis of an exemplary PMLA NIC containing 40% LLL, 2% mPEG, 0.2% mTfR Ab, 0.2% CTLA4 mAb, 0.4% IL-2, and 2% Morpholino AON-HER2/neu. First, a pre-conjugate was synthesized containing 40% LLL, 2% mPEG and 10% of MEA (upper structure). This pre-conjugate was sequentially conjugated with (a) mixture of Mal-PEG3400-TfR Ab and Mal-PEG3400-CTLA4 mAb, (b) Mal-PEG3400-[Ab-(IL-2)], (c) Morpholino AON-HER2/neu (or CK2), and (d) PDP to block remaining free thiol groups to obtain the final product (lower structure).

Referring to FIG. 7, upper structure, a pre-conjugate (P/mPEG/LLL/MEA) was synthesized in a one-pot reaction. PMLA was fully activated with N-hydroxysuccinimide (NHS) in the presence of dicyclohexylcarbodiimide (DCC) in 2 hours. Functional groups including mPEG₅₀₀₀-NH2, H-Leu-Leu-Leu-OH (LLL), and MEA were added sequentially after the completion of each prior amidation. The completion of the reaction was confirmed by thin layer chromatography (TLC; Ninhydrin test). After completion of all reactions (TLC, Ninhydrin test), the unreacted polymer-bound NHS group was decomposed with water. The pre-conjugate was then purified on PD-10 column to remove small molecules, lyophilized and stored at −20° C.

### Synthesis of NIC

Conjugation with AON, Abs, and AbFP.

Synthesis of 3-(2-pyridyldithio)propionyl Morpholino AON (PDP-Morph-AON). Referring to FIG. 7, morpholino-AON 5′-CATGGTGCTCACTGCGGCTCCGGC-3′ [SEQ ID NO: 1] (GeneTools) was designed for the inhibition of human HER2/neu and 5′-CGGACAAAGCTGGACTTGATGTTT-3′ [SEQ ID NO: 2] for inhibition of human and mouse CK2. The 3′-Morpholino-NH2 residue of the AON was conjugated with -succinimidyl-3-(2-pyridyldithio)-propionate (SPDP) and AON-PDP purified using LH-20 column with methanol as eluent. S-succinimidyl-PE G3400-maleimide mAb conjugates were synthesized. Susceptible disulfide bonds of the mAbs (at 1 mg/ml) in phosphate buffer were reduced with 5 mM Tris(2-carboxy ethyl) phosphine hydrochloride (TCEP) followed by purification on PD-10 column to remove free TCEP. The reduced mAbs were conjugated with maleimide-PEG₃₄₀₀-maleimide followed with size-exclusion Sephadex G75 column. Purified mAb(S-succinimidyl-PEG₃₄₀₀-maleimide) was concentrated by diafiltration (30 kDa cutoff) prior to conjugation to preconjugate. Successful conjugation of maleimide-PEG₃₄₀₀-maleimide to mAbs was verified by SEC-HPLC. The synthesized Ab-PEG3400-Mal is used on the same day. The nanoimmunoconjugate PMLA/mPEG/LLL/CTLA-4(PD-1) mAb/TfRmAb/AON was synthesized as follows. Preconjugate P/mPEG/LLL/MEA dissolved in phosphate buffer (pH 6.3, 100 mM) was added to a mixture of mAb-PEG3400-Mal (usually 1 or 2 molecules of each kind of mAb per 1 PMLA molecule) in phosphate buffer (pH 6.3) at room temperature, resulting in the desired stoichiometry, usually 1 or 2 molecules of each kind of mAb per PMLA chain. Complete mAb conjugation was verified by SEC-HPLC. Finally, to a mixture of AON-PDP (each at an equal molar ratio) PMLA/mPEG/LLL/CTLA-4(PD-1) mAb/TfR mAb/MEA was added to conjugate AON via disulfide bond cleavable by glutathione. Unreacted free sulfhydryl groups were blocked with PDP. The final product PMLA/mPEG/LLL/CTLA-4(PD-1) mAb/TfR mAb/AON was purified on size-exclusion Sephadex G75 column.

To understand the synergistic effect between checkpoint inhibitor Abs, AbFP, and AON, 2 subsets of different NICs fully and partially loaded were designed. The NIC versions of Subset 1 was used for treating murine tumors in syngeneic mice and target complete repertoire of immune cells able of responding to all NIC versions. An anti-mouse TfR (mTfR) was used for both BBB transcytosis (metastasis) and for targeting both tumor cells and tumor vasculature (primary breast tumors). The NIC versions of Subset 2 were used for targeting human xenograft tumors in nude mice. Since this model does not have T cells the use of checkpoint inhibitor mAbs was not justified. However, the model was valuable to test anti-tumor activity of IL-2 and IL-12 through NK activation and anti-angiogenic properties of IL-12, as well as dosing. Given the species specificity of anti-TfR Abs, anti-mouse TfR (mTfR) and anti-human TfR (hTfR) mAbs were combined to target both the murine (BBB and tumor vasculature) and human (cancer cells) TfR. The mAbs targeting murine CTLA-4 and TfR were those described herein. To target human TfR, the ch128.1 mouse/human chimeric IgG3 Ab was developed and successfully used to deliver different compounds including viral particles into cancer cells. AON was also used to block CTLA-4 and/or PD-1 by suppressing their synthesis at the mRNA level as was described for HER2/neu receptor. The list of drugs to prevent HER2/neu positive cancer growth is presented in Tables 1 and 2 as follows.

TABLE 1
Exemplary Drugs for Syngeneic mice treatment

| | |
|---|---|
| Group 1 | aCTLA-4 mAb |
| Group 2 | aPD-1 mAb |
| Group 3 | P/mPEG2%/LLL/aCTLA-4/IgG |
| Group 4 | P/mPEG2%/LLL/aCTLA-4/msTfR |
| Group 5 | P/mPEG2%/LLL/aPD-1/msTfR |
| Group 6 | P/mPEG2%/LLL/aPD-1/msTfR + P/mPEG2%/LLL/aCTLA- |
| | 4/msTfR |
| Group 7 | P/mPEG2%/LLL/aCTLA-4/IL-2 |
| Group 9 | P/mPEG2%/LLL/aCTLA-4/IL-12 |
| Group 10 | P/mPEG2%/LLL/aPD-1/IL-2 |
| Group 12 | P/mPEG2%/LLL/aPD-1/IL-12 |
| Group 13 | P/mPEG2%/LLL/aCTLA-4/msTfR/AON-CK2-HER2 |
| Group 14 | P/mPEG2%/LLL/aPD-1/msTfR/AON-CK2-HER2 |
| Group 15 | P/mPEG2%/LLL/AON-CTLA-4/IgG |
| Group 16 | P/mPEG2%/LLL/AON-CTLA-4/msTfR |
| Group 17 | P/mPEG2%/LLL/AON-PD-1/msTfR |
| Group 18 | P/mPEG2%/LLL/AON-PD-1/msTfR + P/mPEG2%/LLL/ |
| | aCTLA-4/msTfR |
| Group 19 | P/mPEG2%/LLL/AON-CTLA-4/IL-2 |
| Group 21 | P/mPEG2%/LLL/AON-CTLA-4/IL-12 |
| Group 23 | P/mPEG2%/LLL/AON-PD-1/IL-2 |
| Group 25 | P/mPEG2%/LLL/AON-PD-1/IL-12 |
| Group 27 | P/mPEG2%/LLL/AON-CTLA-4/msTfR/AON-CK2-HER2 |
| Group 28 | P/mPEG2%/LLL/AON-PD-1/msTfR/AON-CK2-HER2 |
| Group 29 | P/mPEG2%/LLL/aCTLA-4/msTfR/AON-CK2 |
| Group 30 | P/mPEG2%/LLL/aPD-1/msTfR/AON-CK2 |
| Group 31 | P/mPEG2%/LLL/aCTLA-4/msTfR/AON-HER2 |
| Group 32 | P/mPEG2%/LLL/aPD-1/msTfR/AON-HER2 |
| Group 33 | P/mPEG2%/LLL/aCTLA-4/IL-2/AON-CK2 |
| Group 34 | P/mPEG2%/LLL/aPD-1/IL-2/AON-CK2 |
| Group 35 | P/mPEG2%/LLL/aCTLA-4/IL-12/AON-CK2 |
| Group 36 | P/mPEG2%/LLL/aPD-1/IL-12/AON-CK2 |

TABLE 2
Exemplary drugs for xenogeneic mice treatment

| | |
|---|---|
| Group 37 | P/mPEG2%/LLL/msTfR/AON-CK2 |
| Group 38 | P/mPEG2%/LLL/msTfR/AON-HER2 |
| Group 39 | P/mPEG2%/LLL/msTfR/AON-CK2-HER2 |

Optionally, the backbone of PMLA can be labeled with AlexaFluor 680 or other dyes for in vivo imaging or fluorescent microscopy. Estimated average MW was 973 kDa for nanodrugs consisting of 50 kDa PMLA, 2 mAb molecules, 18 AON molecules, 344 LLL molecules and 18 PEG molecules (size about 20 nm).

Physico-Chemical Characterization of NIC.

### Synthesis Monitoring

The preparation of pre-conjugate (P/mPEG/LLL/MEA) was confirmed by TLC for monitoring the completion of each amidation. Each batch of pre-conjugate was verified for pH sensitivity using liposome leakage assay (Ding et al., 2010, Proc Natl Acad Sci USA, 107: 18143-18148, which is incorporated herein by reference as if fully set forth). Thiol residues available for mAb and AON conjugation were assayed by Ellman assay. The successful conjugation of mAb and AON was monitored with SEC-HPLC. Each final NIC was characterized in solution with regard to their size and zeta-potential, using Zetasizer Nano-ZS90 (Malvern). Nanoimmunoconjugates sizes as were on average 20 nm.

Quantitative Analysis of Each Component of Nanoimmunoconjugates in Solution:

Total amount of malic acid was assessed with malate dehydrogenase assay after complete hydrolysis of the nanoconjugate in 6N HCl in sealed ampoule at 116° C. for 16 h (Mossman and Coffman, 1989, Adv Imunol 46: 11-147, which is incorporated herein by reference as if fully set forth). Total amount of PEG was determined by the specific assay (Ding et al., 2015, Int J Mol Sci, 16: 8607-8620, which is incorporated herein by reference as if fully set forth). Total mAb and AON amount was analyzed with a method for simultaneous determination of Ab and AON by selective cleavage of PMLA backbone, which is more reliable for proteins than BCA method. The NIC synthesis process was optimized for reproducible loading of AON and mAb.

In Vitro Biological Activity of NIC

Binding Specificity:

Binding of mAb and AbFP to their antigens were confirmed by ELISA using plates coated with soluble mTfR or hTfR as well as the extracellular domain of HER2/neu (ECD^{HER2}) as was described (Helguera et al., 2006, Vaccine, 24: 304-316; Del Prete et al., 1993, J Immunol, 150: 353-360, both of which are incorporated by reference as if fully set forth). Recombinant mouse CTLA-4-Fc (Biolegend) or PD-1-Fc (R&D Systems) were used to determine binding activity of anti-CTLA-4/PD-1. The binding was also confirmed by flow cytometry or cell-based ELISA using cells expressing the antigens.

Bioactivity Assays of mAbs:

The bioactivity of human IL-2 was determined in proliferation assay using the murine CTLL-2 cell line and that of murine IL-12 in T-cell proliferation assay using human peripheral blood mononuclear cells (PBMC) as was reported (Helguera et al., 2006, Vaccine, 24: 304-316; Ding et al, 2013, J Control Release, 322-339, both of which are incorporated herein by reference as if fully set forth). This latter was possible because although human IL-12 is not active in murine T cells, murine IL-12 is active in human T cells. The ability of IL-12 to induce interferon gamma (IFN-γ) secretion using the murine NK cell line KY-1 was tested and the ability of IL-12 to induce lymphokine activated killer (LAK) cell activation human PBMC as substrates and the human K562 or Daudi cells as preferred targets for LAK cells. Free (non-conjugated) AbFP was used as controls. Effects of anti-mouse anti-CTLA-4 Ab on murine T cells were confirmed by decreased expression of phosphorylated STAT5 and ERK1/2 on Western blots.

Target Protein Inhibition and Cytotoxicity in Cancer Cells:

Cell lines expressing HER2/neu BT-474 (human) and D2F2/E2 (murine) were used as target cells for AON-HER2/neu (human HER2/neu inhibition) and for AON-CK2, AON-EGFR/EGFRvIII (human or murine CK2 inhibition).

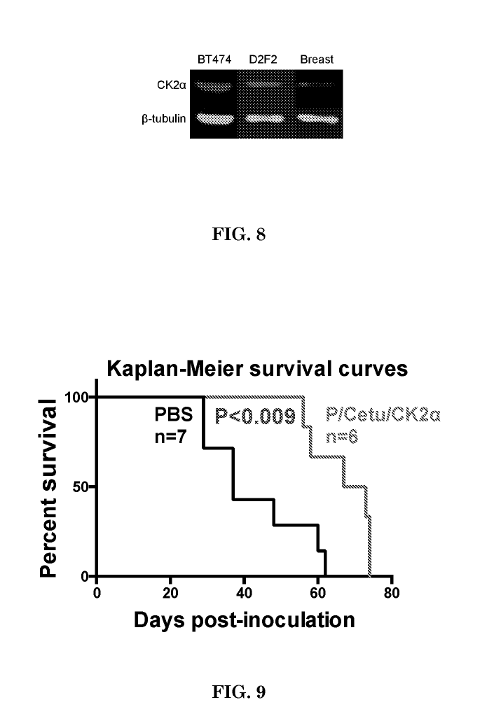

FIG. 8 illustrates Western blot for CK2α and 8-tubulin in human breast cancer BT-474, mouse breast cancer D2F2 and normal human breast tissue. Strong CK2α expression was observed in human breast cancer BT-474, mouse breast cancer D2F2 and low expression was observed in normal human breast tissue.

Referring to FIG. 8, it was found that AON-CK2 targets a consensus sequence 5′-CGGACAAAGCTGGACTTGATG TTT-3′ [SEQ ID NO: 3] of both human and mouse CK2, and that D2F2 cells (only differing from D2F2/E2 by low expression of HER2/neu), similar to the human breast cancer cells such as BT-474, express CK2 at high levels. The ability of different versions of NIC loaded with AON-HER2/neu or AON-CK2 were tested to inhibit proliferation and induce apoptosis (Apopnexin kit, EMD Millipore) in the above mentioned target cells. Given the cell survival role of HER2/neu and CK2 cytotoxicity was observed in all cell lines, with the possible exception of D2F2/E2 in which human HER2/neu was artificially expressed in previously malignant cells (D2F2).

Statistics:

In all studies, samples are tested in triplicate or quadruplicate (depending on the specific test) and experiments repeated 3 times. Statistical analysis will be performed by Student's t-test or ANOVA using Prism5 software (GraphPad Software). p<0.05 is considered significant.

### Example 4—Determining the Efficacy of the Nanoimmunoconjugates in Immunocompetent Mice Bearing Syngeneic Tumors and in Nude Mice Bearing Human Tumor Xenografts

Immunocompetent Mice Bearing Syngeneic Tumors:

This model provides a complete repertoire of immune cells able of responding to all proposed immunostimulators. Given the species specificity of the AbFPs with trastuzumab variable regions, they do not cross-react with murine HER2/neu. To overcome this limitation, the BALB/c or C57 syngeneic murine mammary carcinoma cell line D2F2, expressing human HER2/neu (D2F2/E2), was used. D2F2/E2 grows in immunocompetent BALB/c mice despite the expression of human HER2/neu (highly homologous to the mouse counterpart), a model that has been used to study different immunotherapies including vaccination and passive Ab immunotherapy. In fact, the NIC with an AON confers protection to mice with D2F2/E2, and a NIC loaded with anti-mouse CTLA-4 and anti-mouse TfR Abs also confer protection against the parental cell line D2F2.

Nude Mice Bearing Human Tumor Xenografts:

Although nude (athymic) mice do not have functional T cells and thus, lack adaptive immune response, this model allows the use of human cancer cells expressing HER2/neu that would respond to AON-HER2/neu and AON-CK2 delivery.

It was observed that using xenogeneic mouse models, brain metastases were blocked using nanodrugs that deliver CK2 or HER-2 inhibitors.

FIG. 9 illustrates human brain glioma LN229 growth inhibition by nanoconjugate crossing BBB and blocking CK2α in a xenogeneic animal model. Kaplan-Meier curves show significantly increased (p<0.009, log-rank test) animal survival upon treatment with nanoconjugate with AON to CK2α vs. PBS. Tumor targeting was achieved by cetuximab (Cetu), an EGFR antibody. Median survival was for 70 days vs. 37 days in PBS group. Referring to FIG. 9, it was observed, that the longevity of mice was significantly prolonged when CK2 was blocked, showing a surprising mechanism for this treatment against cancer.

One of the clinically important problems is tumor stem cells. They not only contribute to tumor growth, but also are more resistant to therapies than differentiated cancer cells, and their survival is an important factor of tumor recurrence. For this reason, successful cancer therapies can be directed towards efficient elimination of cancer stem cells.

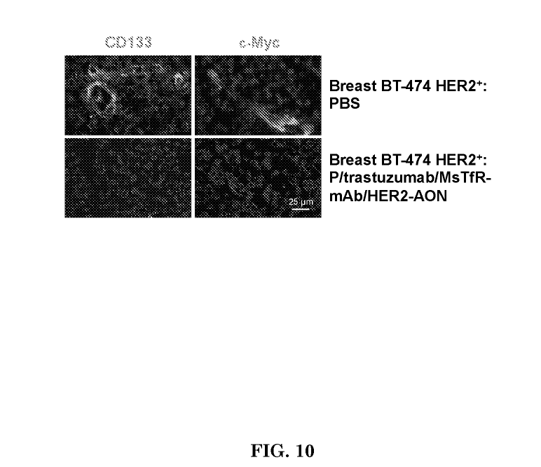

FIG. 10 is a set of photographs illustrating expression of cancer stem cell markers CD133 and c-Myc in BT-474 HER2/neu positive i.c. tumors (brain metastatic model) treated with P/trastuzumab/MsTfR-mAb/HER2-AON and PBS.

High expression of CD133 and c-Myc was observed in PBS-treated tumors and its significant decrease was observed upon treatment with nanodrugs inhibiting CK2α. Nuclei were counterstained with DAPI. Immunofluorescent staining of tissue sections was performed. An immunohistochemical study of treated xenogeneic LN229 brain tumors were conducted using several cancer stem cell markers, CD133, c-Myc, and nestin. All three markers were well expressed in PBS-treated tumors. It was observed that treatment with nanodrugs bearing AON to CK2α caused a dramatic decrease in the expression of all markers.

Checkpoint inhibitor mAbs are not active in nude mice, but the model is valuable to test anti-tumor activity of IL-2 and IL-12 through natural killer (NK) activation and anti-angiogenic properties of IL-12. Human tumor xenografts in nude mice are recommended by FDA to address Ab efficacy against human tumors. As target cells, the human breast cancer cell lines: BT-474 (high levels of HER2/neu), MDA-MB-231 (low HER2/neu), and MDA-MB-468 (no HER2/neu, negative control, positive for EGFR) obtained from ATCC can be used. The inclusion of a HER2/neu negative cell line is particularly attractive given the finding of HER2/neu overexpression on cancer stem cells in HER2/neu negative tumors.

Subcutaneous Tumors:

The advantage of this model using D2F2/E2 or BT-474, widely used in immunocompetent and in nude mice, is the easy monitoring of tumor latency and growth by caliper measurement and by imaging. This model also mimics a s.c. metastasis of breast cancer. Tumors discovered upon orthotopic implantation of cells in the mammary fat pad were also studied.

Tumors in the Brain:

Even though the brain is an immune privileged site with limited access of many systemic immune cells due to the BBB, immune responses can still occur. Brain-resident APC that can travel to peripheral lymph nodes and stimulate T cells that then migrate back to the brain. Microglial cells are brain resident immune regulatory cells and act as APC, expressing the IL-2 receptor upon stimulation, and, thus, can potentially respond to the AbFP stimuli together with T cells. IL-2, IL-12, and AON were designed to have anti-tumor activity in nude mice. Thus, brain tumors as metastatic models were relevant for both animal models. Tumor cells were inoculated intracranially. There are several mouse models for brain metastases (BM) treatment: 1. Environmentally-induced, genetically engineered, including transgenic mutant mice. 2. Intracardiac/intracarotid tumor cell injection that produces both large and small (micro) metastases. 3. Most common intracranial cancer cell inoculation. For BM treatment and BBB delivery of imaging and therapeutic agents, reliable BM models for HER2/neu positive breast cancer with 100% metastasis formation in 2-3 weeks after cancer cell inoculation were developed. The model to achieve this was through intracranial injections, as the other models have low incidence (3-15%) of BM formation.

In all studies, both animal models were used since their different physiology and effector and target cell populations will influence the pharmacokinetics, toxicity, and therapeutic efficacy. Both models complement each other. In terms of tumors, both s.c. and intracranial tumors in all studies were used.

### Methodology

For BALB/c or C57 mice, fully loaded NIC [anti-CTLA-4 (PD-1) mAb, AON-HER2/neu or AON-CK2, anti-HER2/neu Ab-(IL-2) or -(IL-12), and anti-TfR mAb] and partially loaded NIC [anti-CTLA-4 (PD-1) mAb, AON-HER2/neu or AON-CK2, and anti-TfR Ab] were used. In some experiments, free anti-HER2/neu Ab-(IL-2) or -(IL-12) were co-administered. For nude mice, NIC with [AON-HER2/neu or AON-CK2, anti-HER2/neu Ab-(IL-2) or Ab-(IL-12), and anti-TfR mAb] alone or with both AON and anti-TfR mAb, with possible co-administration with free anti-HER2/neu Ab-(IL-2) or Ab-(IL-12) were used. Details in the constructions of experimental NIC and controls to be used in vitro and in vivo are as set forth herein (Conjugation of AON, mAbs, and AbFP; see NIC Subsets 1 and 2).

Studies on pharmacokinetics, tumor targeting, and toxicity were only conducted with the new NIC versions since this information was available for previous versions and for free AbFP. The use of free AbFP was relevant for s.c. and brain tumors. Although the BBB is an obstacle for Ab therapy of brain cancer, brain tumors show some alterations in BBB tight junctions leading to increased permeability. Thus, free AbFP were used to also target brain metastatic breast cancer, igniting an anti-tumor immune response and enhancing the effect of the co-administered NIC. Importantly, since activated T cells can cross BBB, T cells activated in the periphery by the systemic administration of checkpoint inhibitor mAbs could also target intracranial tumors.

To examine mechanisms of action of new NICs, immunohistochemical and western analysis of downstream signaling pathways and cell death were performed. The focus was on pro-survival signaling (phosphorylated Akt vs. total Akt for AON action), activated STAT5 and ERK (for checkpoint inhibitor effects), and tumor cell death (Apopnexin kit staining, and/or cleaved PARP western blot assay). Stem cell marker staining was performed as shown on FIG. 10. It was observed that treatments result in the inhibition of respective signaling and increased death of tumor cells including stem cells.

Anti-tumor efficacy and the mechanisms of drug action was studied by using 8 mice/group in both animal models.

Immunocompetent Mice Bearing Syngeneic Tumors:

Mice were inoculated with 5×10⁵ D2F2/E2 s.c. or other breast cancer cell lines (TNBC) in 0.15 ml Hank's balance salt solution in the right flank (day 0). Either on days 1, 2, and 3 (to mimic micrometastatic s.c. conditions), or on days 6, 7, and 8 (to mimic a well-established tumor), mice were treated with i.v. injections of NIC or controls alone or co-administered with free AbFP. However, other doses and/or schedules may be considered. Tumor growth (s.c.) was monitored with a caliper in two dimensions and the volume calculated according to the formula: tumor volume=(length×width²)/2. Survival was considered as the period of time from tumor challenge until the tumor diameter reaches 1.5 cm when mice were euthanized. Parallel studies were conducted under the same conditions but injecting 10⁴ tumor cells intracranially to mimic brain metastasis.

The tumor sections for tumor infiltrating cells such as NK, CD4 T and CD8 T cells were further stained. Tumors samples were also tested for targeted protein AON inhibition (HER2/neu and CK2) by Western Blot. In addition, spleen cells were isolated and tested in ELISPOT and for Ag-specific CTL using the Calcein AM release assay against D2F2/E2 and D2F2 tumors. Blood samples were analyzed for murine anti-HER2/neu isotype (IgG1/IgG2a) profiling by ELISA as well as murine cytokine profiling using Luminex technology to determine the presence of T_{H}1 and T_{H}2 immune responses (described in Preliminary Data). The approach to inhibit CTLA-4/PD1 without anti CTLA-4/PD1 antibody, but CTLA-4, PD-1 AON Morpholino antisense was used on syngeneic mouse models. Two AON sets for each checkpoint inhibitors were created by GeneTools:

CTLA-4, 5′ to 3′:
[SEQ ID NO: 4]
1. GTCCTCAGGGAGCAGAGTAAAACCC;
and
[SEQ ID NO: 5]
2. TCCAGAAGCCTTGAGATGTGTTTGA.
PD-1, 5′ to 3′:
[SEQ ID NO: 6]
1. TACCTGCCGGACCCACATGCCCAGA;
and
[SEQ ID NO: 7]
2. CCTGGCAGTGTCGCCTTCAGTAGCA.

Nude Mice Bearing Human Tumor Xenografts:

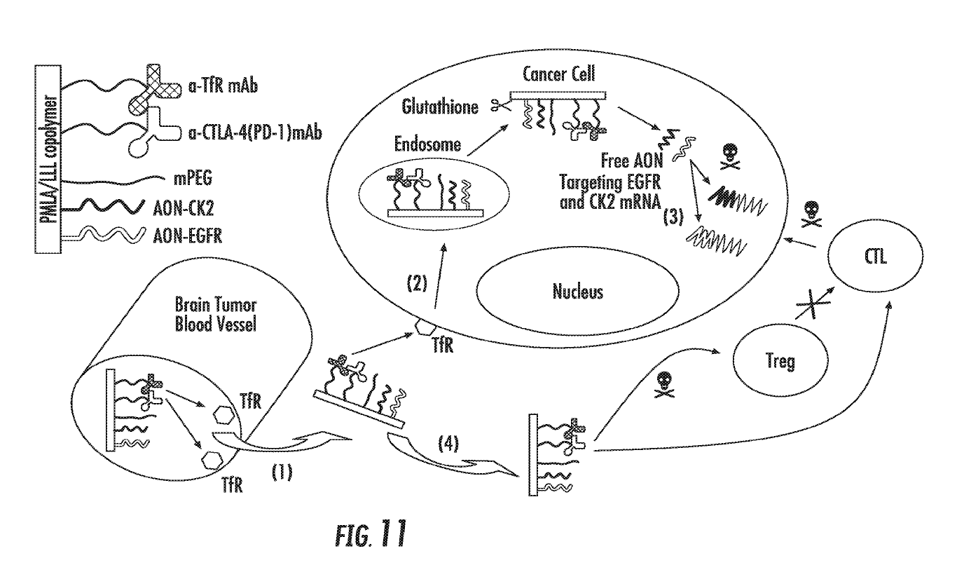

To study the effect of CK2 and HER2/neu AON, BT-474 was selected. Since mice were immunosuppressed, the induction of adaptive immunity was not feasible to study. However, histological and immunohistochemical studies in tumors were conducted to monitor apoptotic/necrotic tumor cells, and tumor infiltrating cells such as NK. AON HER2/neu and CK2 inhibition are assessed by western blot in tumors. Stem cells known for primary and metastatic breast cancer were evaluated after the treatment as a mirror of the treatment effect as was shown for the primary brain cancer with CK2 AON as shown in FIG. 11. PD-1 expression level was associated with the mesenchymal features of breast cancer. Mesenchymal cells have a CD44^{high}/CD24^{low}, vimentin⁺ and E-cadherin⁻ phenotype, whereas epithelial cells normally have CD24^{high}, vimentin⁻, and E-cadherin⁺ phenotype. To understand the mechanism of NIC action, the expression of stem cell markers, CD 44, CD24, CD133, C-myc, notch1 and 3, and nestin, was determined by immunohistochemistry on tumor sections and FACS analysis using fresh tumor cells in vitro and ex vivo after treatment with checkpoint inhibitors and cytotoxic inhibitors for HER2/neu and CK2.

Statistics:

The significance of the differences in blood testing and in tumor volume was determined using the two-tailed Student's t-test or ANOVA for three or more groups, and that in survival (Kaplan-Meier plot) was analyzed by the non-parametric log-rank test.

An alternative of fluorescence imaging analysis in vivo is the PET scan, a highly sensitive and quantitative approach used to study the biodistribution of ¹²⁴I-labeled Abs targeting tumor cells in mice. The MRI for small animals and micro-PET/CT scanners facilities can be used.

In addition to D2F2/E2, it was possible to use the murine cell line expressing human HER2/neu CT26 (CT26-HER2/neu), a carcinoma syngeneic to BALB/c. This model was used to test the efficacy of Abs alone or combined with cytokines and AbFP targeting HER2/neu, including trastuzumab. Similarly, the murine carcinoma cells MC38-HER2/neu syngeneic to C57BL/6 mice was used.

AbFP bearing IL-2, anti-HER2/neu AbFP with IL-2 and IL-12, were used in a nanomedicine. Other anti-HER2/neu AbFP such as IgG3-(GM-CSF) and the bi-functional AbFP (IL-12)-IgG3-(IL-2) and (IL-12)-IgG3-(GM-CSF) were also used for treatments. AbFP were created based on scFv C6MH3-B1. An anti-HER2/neu IgE Ab with scFv C6MH3-B1 variable regions has been developed. This IgE Ab with strong immunostimulatory activity was added.

Pharmacokinetic and Toxicological Studies of NIC

Methodology

Pharmacokinetics and tumor targeting was studied using 8 mice/group in both animal models.

Plasma Drug Concentration, Half-Life Measurement:

Groups of mice not bearing tumors for each NIC variant were injected i.v. with ¹²⁵I-labeled nanoimmunodrug and at designated times blood samples were drawn to measure associated radioactivity using scintillation counting detecting ¹²⁵I-labeled NIC. Half-life was determined by clearance (CL) and volume of distribution (Vd) and the relationship is described by the following equation: t_{1/2}=logₑ 0.5 Vd/CL. Clinical biochemistry testing to evaluate the drug clearance/half life and distribution in tissues and body fluids was performed according to the standard procedures. Fluorescence imaging analysis in vivo: For Alexa Fluor 680-conjugated drug distribution and tumor targeting in vivo was studied using the Xenogen IVIS 200 Imaging System.

Toxicology studies were performed using 8 mice/group in both animal models. Mice were injected i.v. in 160 μl, in the tail vein with NIC. Doses in the range of 2-5 mg/kg body weight equivalent to the loaded Abs were tested. The NIC in the dose range of 5 to 25 mg/kg body weight were tested. These doses cover the range commonly used for most Abs and their derivatives in the clinic. The goal was to find powerful and effective NCI treatment dose, however non-toxic, that can be used in tumor efficacy studies in both mouse models. Animals were checked regularly for clinical manifestations of toxic effects including: activity (hyper or hypo); slow moving; loss of interest; nutrition; neurological score: (Grade 1: tail weakness or tail paralysis; Grade 2: hind leg/limb paresis or hemiparesis; Grade 3: hind leg/limb hemiparalysis; Grade 4: complete paralysis (tetraplegia), moribund stage or death. Blood biochemical analyses were routinely performed as follows: transaminases (AST, ALT)—liver function; bilirubin (direct, indirect); creatinin; blood urea nitrogen; blood: white blood cells; red blood cells; platelets; hemoglobin; and inflammation: C-reactive peptide. At the end of the experiment, animals were euthanized and organs subjected to macro- and microscopic examination.

Treatment data were developed with CTLA-4 and PD-1 Ab concentration of 5 mg/kg that allow using escalating dosages of 3, 5, and 10 mg/kg. However, concentrations of anti-PD-1 at 1 mg/kg and CTLA-4 at 3 mg/kg were also used due to relatively high toxicity. Decreased concentrations of Treg mAbs as part of NIC were also explored with all appropriate evaluations. Lower concentrations allowed reducing toxicity known from clinical use without compromising the anti-tumor effects.

### Example 5—Treatment Design for Brain Cancer

The strategies for blocking CTLA-4 and PD-1 including use of antibodies against these targets for treating breast cancer described in Example 1 are also applicable for treating brain cancer.

Moreover, treatment of gliomas with combination of these antibodies was not successful because as other antibodies they do not cross the BBB. Upon this treatment, only systemic immune response was activated.

The present disclosure provides, in part, nanoimmunotherapeutics that are able to pass through the endothelial system and the BBB, and deliver drugs and immunostimulatory antibodies directly to the tumor and to the immune cells in its microenvironment, thus activating the general immune response together with brain tumor local immune response.

An efficient approach for brain cancer treatment was developed using nanoimmunoconjugates for (1) dual (systemic and local) stimulation of anti-tumor immune response and (2) inhibition of tumor cell proliferation through blocking the synthesis of common GBM targets CK2 and EGFR. Local immunostimulation is important for GBM because of brain immune privilege and lack of efficacy of CTL-activating mAbs against brain cancer as they cannot pass through BBB (Muldoon et al., 2013, J Cereb Blood Flow Metab, 33:13-21 and Oh et al., 2014, J Transl Med, 12:107, both of which are incorporated herein by reference as if fully set forth). This strategy takes advantage of BBB-crossing nanoimmunoconjugates to boost anti-tumor response by removing functional constraints imposed on CTLs by Treg using mAbs to CTLA-4 and/or PD-1. The targeted cancer treatment combines multiple therapeutic agents in one nanoimmunoconjugate. The main advantages are as follows. The cancer treatment achieved by the ability of nanoimmunoconjugates of boosting local and systemic anti-cancer immunity in immune privileged brain tumors. The BBB-crossing nanoimmunoconjugate is engineered by the polymeric linear platform for targeting tumors. The nanoimmunoconjugate bears CTL-activating mAbs to CTLA-4 or PD-1 to boost local and systemic anti-tumor response, together with drugs blocking GBM proliferation by inhibiting CK2, and both wild-type EGFR and mutated EGFRvIII, the two major cancer markers of gliomas. As a result, it is able to eradicate brain tumors directly and through the immune cytotoxic response. Combining active cytokines (e.g., IL-2) stimulating local CTL anti-tumor response and mAbs stimulating systemic and local anti-tumor immunity on one nanoimmunoconjugate is advantageous for GBM therapy.

FIG. 11 is a schematic drawing illustrating the effects (mechanism of action of combination therapy when the anti-tumor immune response is activated, together with inhibition of tumor specific molecular markers, EGFR and CK2) of a nanoimmunoconjugate that includes a PMLA backbone, LLL, a TfR mAb, a-CTLA-4 (PD-1), AON-CK2, and AON-EGFR on brain tumors. Referring to FIG. 11, in step (1) the nanoimmunoconjugate binds to mouse transferrin receptor (TfR) enriched on tumor cells and endothelium, and gets transcytosed through BBB to the tumor interstitium. In step (2), the nanoimmunoconjugate binds to TfR on mouse GBM and gets internalized. It proceeds to the endosome, disrupts its membrane, the drug is released in the cytoplasm and AONs are cleaved off by cytoplasmic glutathione. In step (3), free AON inhibits target (EGFR and/or CK2) translation and causes cancer cell death. In step (4), the nanoimmunoconjugate that cannot enter the cancer cells binds to and inactivates Treg through CTLA-4(PD-1) antibody removing their block on CTL, which allows the CTL to attack and kill cancer cells.

Nanoimmunoconjugates were observed to pass through BBB by active targeting and provide simultaneous specific brain cancer cell killing (internal attack) and stimulation of anti-tumor immune response (external attack), which significantly increased anti-tumor efficacy.

### Example 6—Synthesis and In Vitro Characterization of Nanoimmunoconjugates for Treatment of Brain Cancer

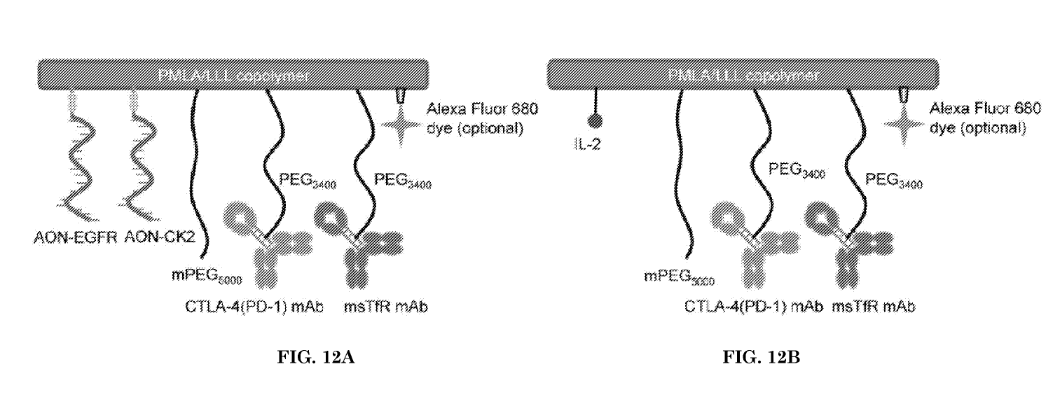

Intracranial brain tumor-bearing mice was used as a model for testing efficiency of the PMLA-based nanoimmunoconjugates for brain tumor treatment. FIGS. 12A-12B are schematic drawings of the PMLA-based nanoimmunoconjugates designed for syngeneic mouse models. FIG. 12A illustrates a nanoimmunoconjugate containing a PMLA-backbone, LLL, mPEG, CTLA-4(PD-1) mAB, msTfR mAb, AON-EGFR, AON-CK2, and optionally Alexa Fluor 680 dye designed for suppression of tumor cell growth by blocking EGFR and CK2 with AON. Tumor targeting was achieved by anti-msTfR mAb. Treg targeting was achieved by anti-CTLA4 or PD-1 mAb. Optional fluorescent dye allowed conjugate detection in cells and tissues including live imaging. FIG. 12B illustrates an immunostimulatory nanoimmunoconjugate containing a PMLA-backbone, LLL, mPEG, CTLA-4(PD-1) mAB, msTfR mAb with attached active cytokine (IL-2) for additional immune stimulation and optionally Alexa Fluor 680 dye. For xenogeneic mouse models, anti-lymphocyte mAb was substituted by anti-human TfR to target tumor cells.

It was observed that the formulation of Polycefin with AON to laminin-411 inhibited tumor angiogenesis. PMLA-based drug delivery system targeting frequent GBM markers EGFR and CK2, was utilized in nanoimmunoconjugates. These compounds mount a double attack on brain tumor cells, that is, blocking their growth by AON and boosting anti-tumor immune response. Another advance was to combine AON with active anti-tumor cytokines (e.g., IL-2) on the nanoplatform to increase anti-tumor effect. The polymalic acid based drug delivery system is advantageous as a scaffold because it (1) can cross BBB, (2) can be modifiable to attach additional drug moieties including AONs and mAbs, and (3) can to specifically target brain tumors. The PMLA-based nanoimmunoconjugates could simultaneously carry AONs to different tumor targets (CK2 and EGFR), and functional antibodies, thereby increasing the efficacy of tumor suppression. By simultaneously increasing CTL response to glioma, and killing tumor cells by targeting EGFR and CK2, it was possible to develop the surprisingly efficacious nanotherapy for brain cancer with low if any systemic toxicity. Nanoimmunoconjugates variants were synthesized, thorough physico-chemical characterization and synthesis optimization was provided. The variants were tested in tumor cells in vitro.

Nanoimmunoconjugates were characterized for purity (free of endotoxin or contaminating other material), and by HPLC, spectrophotometry, ELISA, and new detailed quantitative chemical and imaging analysis (Ljubimov et al., 2004, Invest Ophtalmol Vis Sci, 45: 4583-4591, which is incorporated herein by reference as if fully set forth). In vitro cell viability was tested to select out toxic nanoconjugates. In vitro measurement of inhibition of the target proteins (CK2, EGFR) and functional assays of anti-CTLA-4, and PD-1 antibodies and cytokines were performed by Western blot analysis, immunohistochemistry, ELISA, FACS, and apoptosis assays.

GBM Cells Express CK2 and EGFR, which were Downregulated by Nanodrugs

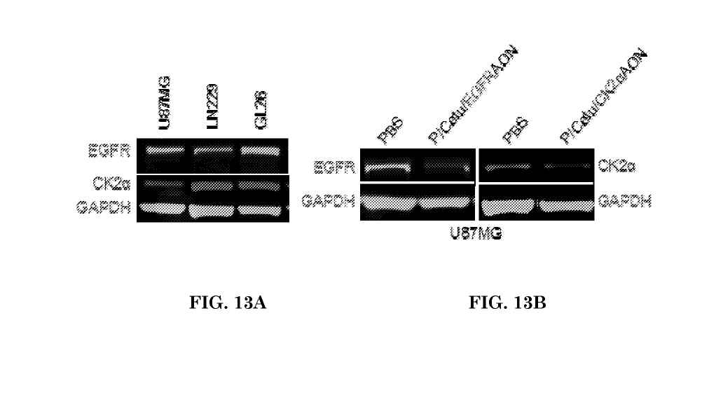

FIGS. 13A-13B are photographs of Western blots showing EGFR and CK2α expression in GBMs and their inhibition by nanodrug-conjugated AONs. FIG. 13A illustrates that both EGFR and CK2α were expressed in three cell lines U87MG, LN229, and GL26. FIG. 13B illustrates that compared to PBS, the expression of EGFR and CK2α was markedly reduced upon cell treatment with P/Cetu/EGFR-AON (left panel) and P/Cetu/CK2α-AON (right panel) using anti-EGFR mAb cetuximab (Cetu) for cellular uptake. GAPDH was used a housekeeper to normalize gel loading for Western blots. Referring to FIG. 13A, it was observed that both proteins were expressed in human (U87MG and LN229) and mouse (GL26) GBMs. Referring to FIG. 13B, it was observed that PMLA-based nanodrug with anti-EGFR cetuximab mAb targeting cancer cells effectively inhibited both CK2 and EGFR expression with respective AON having sequences of SEQ ID NO: 2 and 8.

Experimental Design

Nanoimmunoconjugates were synthesized as described herein. In vitro function of pre-selected AON cross-reacting with human and mouse were tested in human U87MG, LN229, and mouse GL26/G1261 GBM cultures, and compared to normal HAST 40 astrocytes. CK2 and EGFR inhibition were confirmed by Western analysis. ELISA, binding/FACS and proliferation assays tested the activity of function-blocking mAbs to CTLA-4 and PD-1, as well as of IL2 and/or IL-12 as described (Peggs et al., 2009, J Exp Med, 206: 1717-1725, which is incorporated herein by reference as if fully set forth). Cell death after treatments was assessed by Apopnexin assay (EMD Millipore). Chemical and functional complexities were tested by specific quantitative assays (Ljubimova et al., 2014, J Vis Exp, 88, and Ding et al, 2015, Int J Mol Sci, 16: 8607-8620, both of which are incorporated herein by reference as if fully set forth). Data were statistically analyzed in the Cancer Center Biostatistics core. In vitro experiments were routinely performed in triplicate, with relevant specificity controls.

Preparation of Pre-Conjugates

Because the nanoimmunoconjugates contain multiple components, the success and reproducibility of the synthesis was monitored. A sequential synthesis procedure with controlled conjugation of each component was developed, and verification of each step by SEC-HPLC was performed (Lee et al., 2006, Bioconjug Chem, 17: 317-326, and Fujita et al, 2007, J Control Release, 122: 356-363, both of which are incorporated herein by reference as if fully set forth). PMLA for drug synthesis was produced and purified from myxomycete Physarum polycephalum. Purified PMLA was characterized prior to synthesis of nanoimmunoconjugates. PMLA of m.w. (weight-averaged molecular weight)=100 KDa (polydispersity P=1.1) was prepared by fractionation on Sephadex G25 fine.

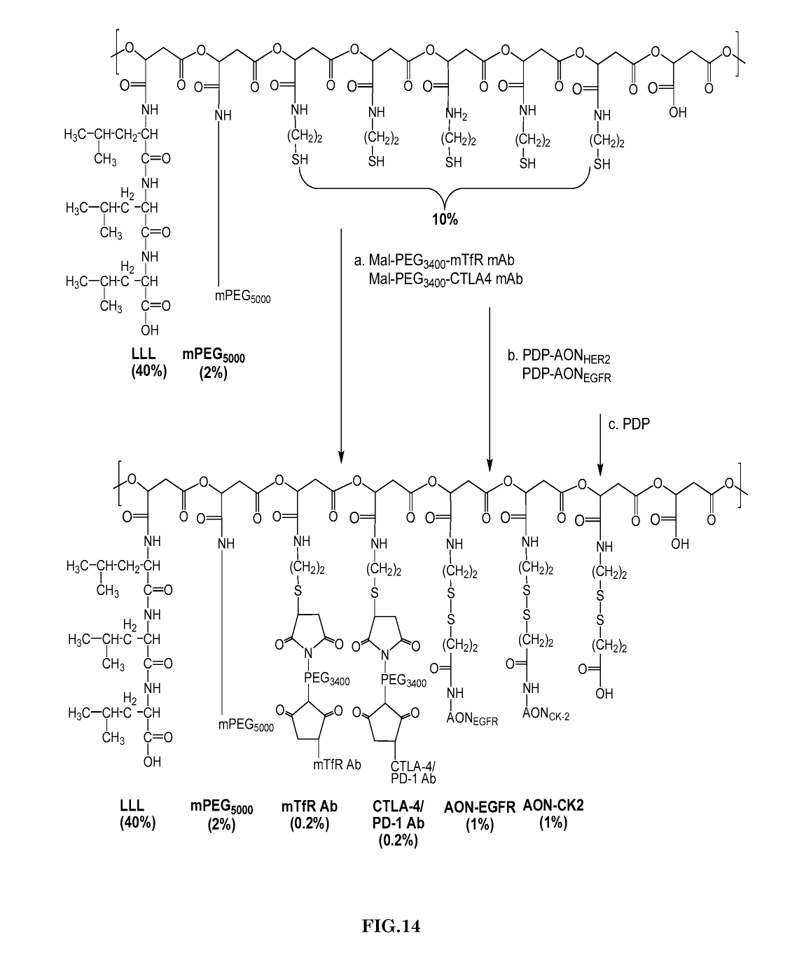

FIG. 14 illustrates the synthesis of an exemplary nanoimmunoconjugate that contains a PMLA backbone, 40% LLL, 2% mPEG, 0.2% TfR Ab, 0.2% CTLA-4/PD-1 Ab, 1% AON-EGFR, and 1% AON-CK2α. First, a pre-conjugate was synthesized with 40% LLL, 2% mPEG and 10% MEA (upper structure). It was sequentially conjugated with (a) mixture of Mal-PEG3400-anti-TfR mAb and Mal-PEG3400-anti-CTLA-4 or anti-PD-1 mAb, (b) mixture of PDP-AON-CK2, PDP-AON-EGFR, and (c) PDP to block remaining free thiol groups to obtain the final product (lower structure). Referring to FIG. 14, upper structure, first, a pre-conjugate (P/mPEG/LLL/MEA) was synthesized in a one-pot reaction. PMLA was fully activated with NHS in the presence of DCC for 2 hr. Functional groups including mPEG5000-NH₂, H-Leu-Leu-Leu-OH, and MEA (2-mercapto-1-ethylamine) were added sequentially after completion of each prior amidation confirmed by thin layer chromatography (TLC; Ninhydrin test). Unreacted polymer-bound NHS group was decomposed with water. The pre-conjugate was purified on PD-10 column to remove small molecules, lyophilized and stored at −20° C.

### Synthesis of Nanoimmunoconjugates (P/mPEG/LLL/Anti-msTfR mAb/Anti-CTLA-4 mAb or Anti-PD-1 mAb/AON(CK2, EGFR)

Synthesis of 3-(2-pyridyldithio) propionyl Morpholino AON (PDP-Morph-AON) is described herein as an example. Morpholino-EGFR-AON: 5′-TCGCTCCGGCTCTCCCGATCAATAC-3′ [SEQ ID NO: 8] and CK2α-AON: 5′-CGGACAAAGCTGGACTTGATG TTT-3′ [SEQ ID NO: 3] were from Gene Tools and had been functionally verified for efficacy with nanoimmunoconjugates as shown in FIG. 13B. The 3′-NH₂ AON terminus was conjugated with succinimidyl-3-(2-pyridyldithio)-propionate (SPDP) and PDP-AON purified on LH-20 column with methanol as eluent. The PDP-AON was stored at −20° C. IL-2 was conjugated with SPDP similar to AON and purified on PD-10 column to obtain IL-2-PDP.

### Synthesis of S-succinimidyl-PEG3400-maleimide mAb Conjugates

Susceptible mAb S—S bonds were reduced in phosphate buffer with 5 mM Tris(2-carboxy ethyl) phosphine hydrochloride (TCEP) at room temperature (RT) for 30 min and free TCEP was removed on PD-10 column. mAbs were conjugated with maleimide-PEG3400-maleimide (mPEGm) for 30 min at RT followed with size-exclusion on Sephadex G75 column to remove unreacted mPEGm. Purified mAb(S-succinimidyl-PEG3400-maleimide) was concentrated by diafiltration (30 kDa cutoff) prior to conjugation to the preconjugate. Conjugation of mPEGm to mAbs was verified by SEC-HPLC. The synthesized mAb-PEG3400-Mal was used on the same day.

### Synthesis of Full Nanoconjugate P/mPEG/LLL/Anti-msTfR mAb/Anti-CTLA-4 mAb or Anti-PD-1 mAb/AON(CK2, EGFR/EGFRvIII)

Preconjugate P/mPEG/LLL/MEA in phosphate buffer (pH 6.3, 100 mM) was added to a mixture of mAb-PEG3400-Mal containing an equivalent of 2 molecules of each mAb per PMLA molecule, in phosphate buffer pH 6.3 at RT. Conjugation of mAbs was verified by SEC-HPLC. Then, PMLA/mPEG/LLL/anti-msTfR mAb/anti-CTLA-4 mAb or anti-PD-1 mAb/MEA was added to an equimolar mixture of PDP-AONs by forming S—S bonds. Similarly, IL-2-PDP was attached to the nanoconjugate. Unreacted free sulfhydryl groups were blocked with PDP. The final drug P/mPEG/LLL/anti-msTfR mAb/anti-CTLA-4 mAb or anti-PD-1 mAb/AON(CK2,EGFR) was purified on Sephadex G75 column.

IgG and scrambled AON were standard negative controls. Anti-mouse TfR mAb was used for BBB transcytosis and tumor cell targeting. Nanoimmunoconjugates for treating human xenograft tumors in nude mice shown Tables 3 and 4 have no anti-CTLA-4 mAb or anti-PD-1 mAbs, as this model does not have T cells. However, the model was used to test IL-2 or IL-12 anti-tumor activity through activation of NK and anti-angiogenic property of IL-12. Given species specificity, anti-msTfR to target murine BBB was combined with anti-huTfR to target human cancer cells. CTLA-4, PD-1 and TfR mAbs were described in herein. For imaging, PMLA was labeled with Alexa Fluor 680 dye (Inoue et al., 2012, PLoS One, 7: e31070, which is incorporated herein by reference as if fully set forth). Estimated average MW is 973 kDa for nanodrugs consisting of 100 kDa PMLA, 2 mAb molecules, 18 AON molecules, 344 LLL molecules and 18 PEG molecules.

TABLE 3
Nanoimmunoconjugates for syngeneic mouse treatment

P/mPEG/LLL/CTLA-4/mTfR/AON** (fully
loaded)
P/mPEG/LLL/CTLA-4/IgG/AONs
P/mPEG/LLL/mTfR/CTLA-4/AON-scrambled
P/mPEG/LLL/CTLA-4
P/mPEG/LLL/mTfR/CTLA-4/IL-2
P/mPEG/LLL/IgG
P/mPEG/LLL/PD-1/mTfR/AON (fully loaded)
P/mPEG/LLL/PD-1/IgG/AONs
P/mPEG/LLL/mTfR/PD-1/AON-scrambled
P/mPEG/LLL/PD-1
P/mPEG/LLL/mTfR/PD-1/IL-2
**anti-mouse AON-EGFR/EGFRvIII or AON-CK2;
LLL, trileucine;
IgG and AON-scrambled are negative controls

TABLE 4
Nanoimmunoconjugates for xenogeneic mouse treatment for
dosing and toxicity studies

P/mPEG/LLL/mTfR/hTfR/AON** (fully
loaded)
P/mPEG/LLL/mTfR/hTfR/AON-scrambled
P/mPEG/LLL/AON
***anti-human AON-CK-2 and AON-EGFR/EGFRvIII;
LLL, trileucine;
IgG and AON-scrambled are negative controls.

### Physico-Chemical Characterization of Nanoimmunoconjugates

### Synthesis Monitoring

The preparation of pre-conjugate (P/mPEG/LLL/MEA) was confirmed by TLC to monitor the completion of each amidation. Each batch of preconjugate was verified for pH-sensitivity using liposome leakage assay. Successful mAb and AON conjugation was monitored by SEC-HPLC. Each nanoimmunoconjugate's size and zeta-potential were characterized in solution using Zetasizer Nano-ZS90 (Malvern). Sizes of the nanoimmunoconjugates, such as shown on FIGS. 12A-12B, were on average 20 nm.

Quantitative Analysis of Each Nanoimmunoconjugate Component in Solution

Total malic acid was assessed with malate dehydrogenase assay after complete nanodrug hydrolysis in 6N HCl in sealed ampoule at 116° C. for 16 hr. The amount of PEG was determined by a specific ammonium ferrothiocyanate assay. mAb and AON content was analyzed by a method for simultaneous determination of mAb and AON after selective cleavage of the PMLA backbone with ammonium hydroxide.

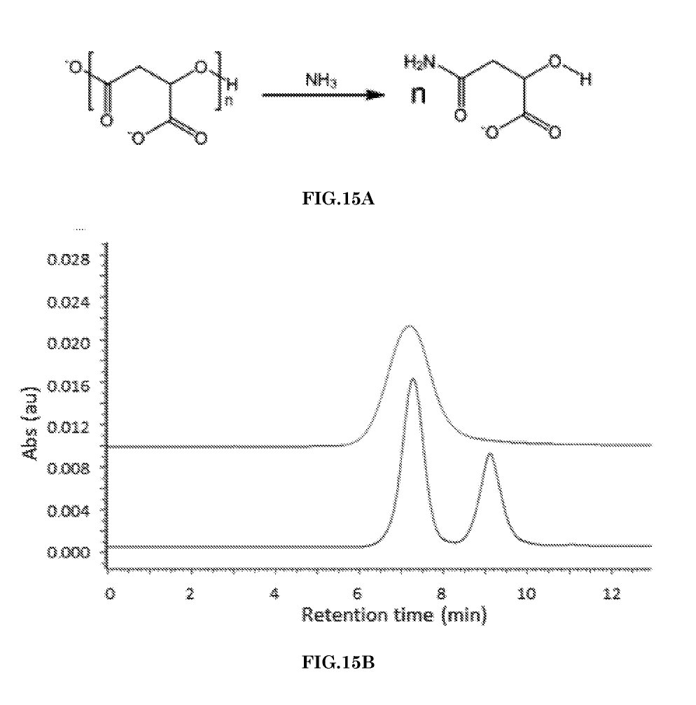

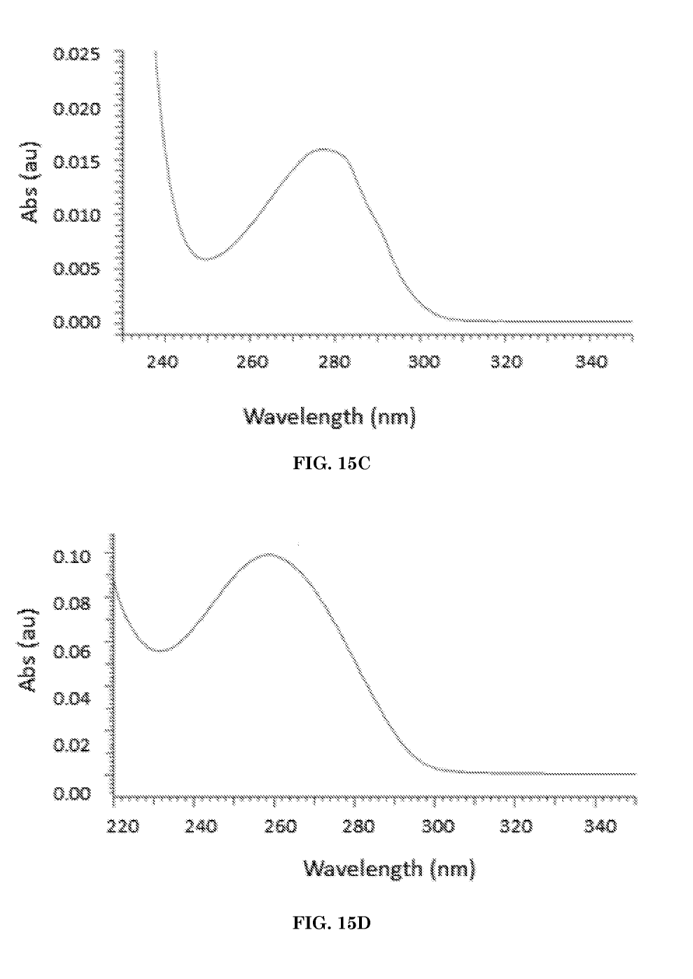

This method allows quantifying mAb and AON in nanoconjugate together using SEC-HPLC. FIGS. 15A-15D illustrate selective cleavage of a PMLA nanoimmunoconjugate. FIG. 15A is a schematic drawing of selective cleavage of the PMLA nanoconjugate by ammonia. FIG. 15B is an HPLC profile of the PMLA nanoimmunoconjugate before (upper curve) and after cleavage (lower curve). Referring to this figure, the PMLA nanoimmunoconjugate was first analyzed before cleavage (upper curve) with SEC-HPLC shown as a single broad peak and after cleavage (lower curve) shown as two separated peaks. FIG. 15C is a profile of the first peak identified as mAb with maximum spectrum wavelength of 280 nm. FIG. 15D is a profile of the second peak identified as AON at 260 nm. It was reported that cleavage does not affect mAb and AON integrity and biological activity (Ding et al., 2015, Int J Mol Sci, 16: 8607-8620, which is incorporated herein by reference as if fully set forth). In contrast to bicinchoninic acid (BCA) method for assessing antibody amount, a method described herein yielded consistently reliable results. ELISA data demonstrated that mAb function was not appreciably affected during conjugation to PMLA platform. Liposome/calcein fluorimetric assay was conducted to assess membrane disrupting activities of nanoimmunodrugs. It was observed that this assay was more reliable than the hemolytic test previously performed, as the results were not obscured by interactions of nanoimmunoconjugaes with red blood cell proteins.

Test of AON Releasing Module of Nanoimmunoconjugates

In quality controls, the activity of AON releasing module and the amount of AON binding to nanoimmunodrugs were assessed. Nanoimmunoconjugates (0.25 mM bound AON) were incubated with 5 mM GSH (γ-L-glutamyl-L-cysteinylglycine), in 50 mM phosphate buffer pH 7.4 at 37° C., and the reactions at various times until completion were stopped with 20 mM N-ethylmaleimide. The liberated reduced AONs were detected as N-ethylmaleimidyl derivatives by SEC-HPLC, A₂₆₀. Complete release was obtained in the presence of 50 mM dithiothreitol (DTT) and was 100% complete by 60 min at 37° C.

Analysis of Function and Conjugation of mAbs to Nanoimmunoconjugates Variants

The reaction mixtures were carefully purified by SEC-HPLC and antibody attachment verified by SDS-PAGE. Quantitative testing of the IgGs ratio was done by ELISA with available antibodies. Antibody activity and presence of two mAbs on nanoimmunodrugs was determined by pull-down ELISA using immobilization of nanoimmunodrugs with one mAb and testing with specific antibodies for another mAb or IL protein(s). Time course of drug accumulation in cells was monitored by confocal microscopy and target inhibition, by Western blotting. The bioactivity of IL-2 was determined in proliferation assay using murine CTLL-2 cell line, and that of murine IL-12, in T-cell proliferation assay with human peripheral blood mononuclear cells (PBMC). This was possible because murine IL-12 was active in human T cells. The ability of IL-12 to induce interferon gamma (IFN-γ) secretion in murine NK cell line KY-1 was tested, as well as the ability of IL-12 to induce lymphokine activated killer (LAK) cell activation in human PBMC as substrates and human K562 or Daudi cells as targets for LAK cells. Effects of anti-mouse anti-CTLA-4 and anti-PD-1 mAbs on murine PBMC were confirmed by proliferation assay and decreased phosphorylation of STAT5 and ERK1/2 on Western blots as described (Harvill and Morrison, 1995, Immunotechnology, 1: 95-105; Comin-Anduix et al., 2010, PLoS One, 5: e12711; Liston and Kim, 2009, Immunol Cell Biol, 87: 443-444; and Kramerov et al., 2011. Mol Cell Biochem, 349: 125-137, all of which are incorporated herein by reference as if fully set forth). Serum IL-2 and IL-12 levels were measured by Luminex assay. Mice with GL26 brain tumors were treated I.V. 5 times with naked mAbs, mAbs on nanoimmunoconjugate or a combination of nanoimmunoconjugates. It was observed that only polymer-attached mAbs prolonged animal survival because (1) nanoimmunodrug was able to cross BBB, and (2) nanoimmunodrug activated tumor local immune response whereby anti-CTLA-4/PD-1 mAbs block Treg from preventing CTL to attack brain cancer cells inside the tumor as shown on FIG. 11.

Analytical Methods

The substitution of N-hydroxysuccinimidyl (NHS) residues was followed by RP-HPLC analysis of the reaction mixtures. Thiol residues were assayed by Elman's method after removal of free 2-MEA by diafiltration (5 kDa cutoff). Amounts of maleimido groups were quantified by their reaction with 2-MEA and back titration using Elman's method. Amino acids were quantified by RP-HPLC after hydrolysis of conjugates in 6 M HCl at 100° C. and colorimetry with trinitrofluorobenzene (TNBS) following standard protocols. Reducing SDS-PAGE on 10% polyacrylamide gels and Western analysis were carried out as described (Ljubimova et al., 2013, J Drug Target, 21: 956-967, which is incorporated herein by reference as if fully set forth).

Culture of Glioma Cells

Human GBM cell lines U87MG and LN229 (from ATCC), and mouse GL26 and GL261 lines were used. Cells were grown as described (Ding et al., 2010, Proc Natl Acad Sci USA, 107: 18143-18148, which is incorporated herein by reference as if fully set forth). Treatment with nanoimmunodrug variants was performed. AON-scrambled variants and PBS (drug solvent) served as controls. Experiments were performed in triplicate and repeated at least three times, with appropriate statistical analysis as described herein.

In Vitro Cell Viability Tests

Viable cells were quantified by CellTiter 96 AQueous MTS Assay (Promega). Cell death was assayed by Apopnexin kit (EMD Millipore) as recommended by the manufacturer.

Statistics

Whenever appropriate, quantitative data from different groups were compared statistically using Prism5 program (GraphPad Software). For two groups, Student's t test was used, for three and more groups, two-way ANOVA with appropriate post-hoc tests was used.

### Example 7—Examination of Inhibitory Effects of Nanoimmunoconjugates on Brain Tumor Growth

Preclinical in vivo efficacy was assessed, to select lead nanoimmunoconjugates and treatment regimens. Syngeneic mouse models of GL26 and GL261 intracranial glioma, and xenogeneic models of human intracranial U87MG and LN229 GBMs in nude mice were used. Data showed feasibility of these models for assessing therapeutic efficacy of nanodrugs. In these intracranial models BBB functioned strongly, precluding free antibody and other drugs from reaching brain tumor (Agarwal et al., 2013, Drug Metab Dispos, 41: 33-39, which is incorporated herein by reference as if fully set forth). An important advantage of this system is simultaneous action of AON drugs on tumor cells and immune system stimulation provided by all-in-one nanoimmunodrug that has not been used before in nanomedicine. Another advantage of the system is its ability to pass through BBB, as local immune stimulation appears to be critical for brain tumor treatment. Data showed promise of targeted nanodrugs blocking CK2 and EGFR to treat brain gliomas. Nanoimmunoconjugates blocking EGFR and CK2 in tumor cells was observed to significantly suppress brain tumor growth and increase animal survival, and that this effect was markedly enhanced by simultaneous stimulation of local and systemic anti-tumor immune response with mAbs to CTLA-4 and PD-1, and/or nanoimmunodrug-attached active cytokine IL2 for additional tumor immune modulation.

GBM Suppression by Nanodrugs Targeting EGFR and/or CK2.

Referring to FIGS. 13A-13B, nude mice with human intracranial GBMs LN229 and U87MG were treated I.V. six times with nanodrugs effective in vitro. It was observed that treatment resulted in a near doubling of animal survival compared to PBS injections.

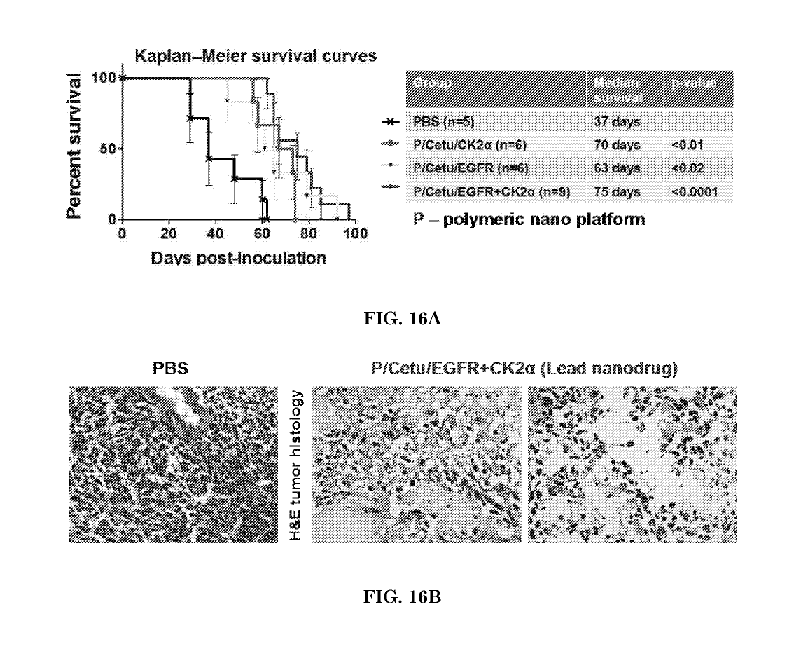

FIGS. 16A-16B illustrate that nanoimmunoconjugates containing AONs specific to EGFR and/or CK2α inhibited LN229 GBM growth and prolonged tumor-bearing animal survival. FIG. 16A (left) is a set of Kaplan-Meier curves showing animal survival upon treatment with nanoimmunoconjugates P/Cetu/AON-CK2α (closed square), P/Cetu/AON-EGFR and P/Cetu/AON-CK2α/AON-EGFR compared to control treatment with PBS (x-mark), and (right) is a table showing quantitation of median survival. It was observed that treatment of animals with AON to CK2α, EGFR, and their combination on one nanoimmunoconjugate targeted to the tumor by Cetuximab (Cetu) significantly increased survival of the animals compared to the treatment with PBS. FIG. 16B are photographs of tumor morphology following treatments with nanoimmunoconjugates and PBS. It was observed that PBS-treated tumors are well developed; nanodrug-treated ones have large necrotic areas. Referring to FIG. 16A, it was observed that inhibition of CK2 or EGFR was similarly effective. Their combination on one nanodrug produced a small increase in survival shown for LN229. Similar results were obtained for U87MG GBM. Histological H&E analysis revealed florid tumor growth in PBS-treated animals, whereas nanodrug-treated tumors had large areas of necrosis. Labeled nanodrug was detected inside tumor cells attesting to its ability to cross BBB.

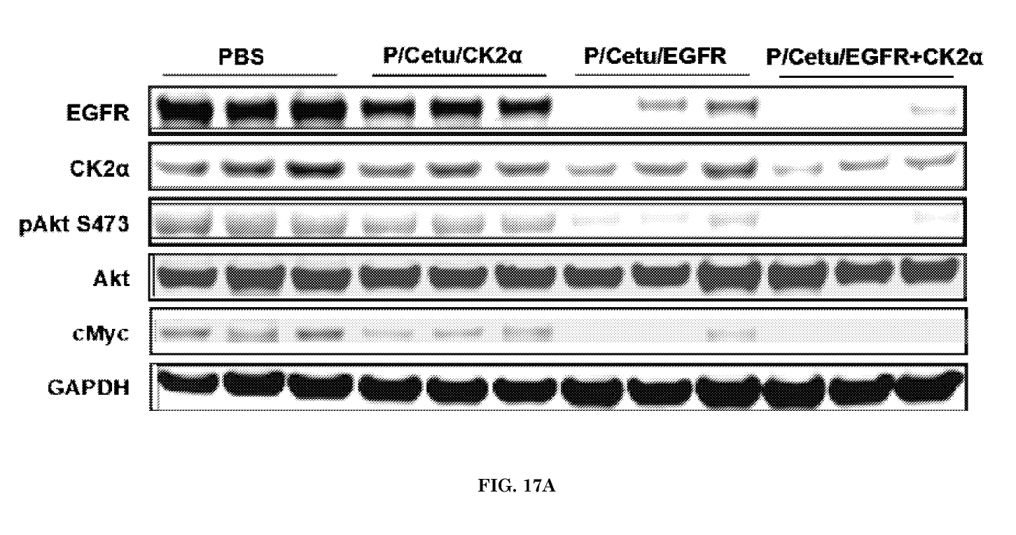

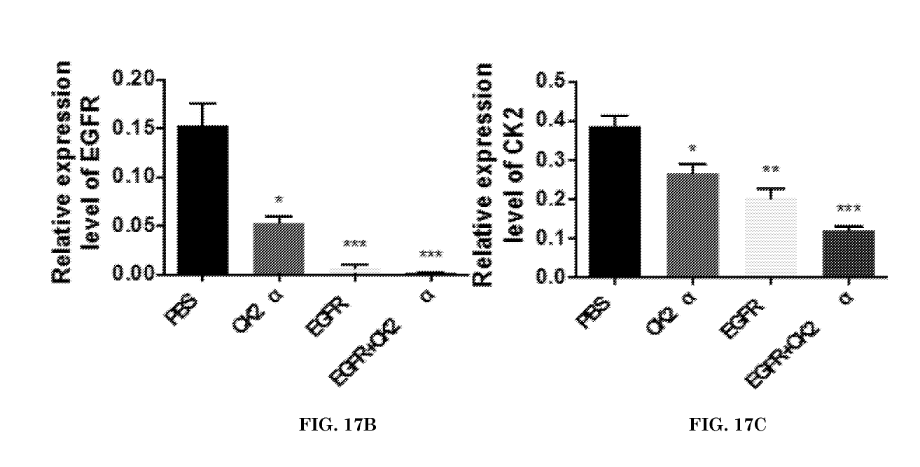

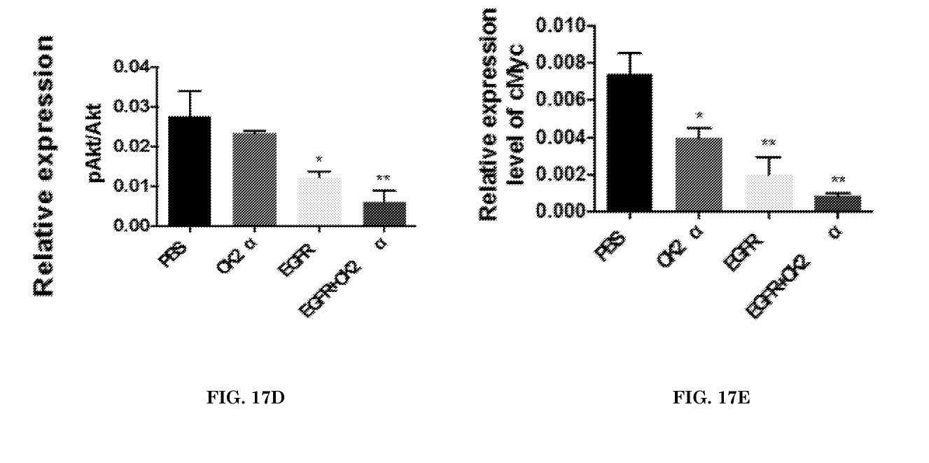

FIGS. 17A-17E illustrate effects of nanoimmunoconjugates P/Cetu/AON-CK2α, P/Cetu/AON-EGFR, and P/Cetu/AON-EGFR/AON-CK2α on pro-survival and proliferative signaling in intracranial LN229 xenogeneic tumors compared to control treatment with PBS. FIG. 17A is a set of photograph of Western blots showing reduction of EGFR, CK2α, as well as of phosphorylated/activated Akt (pAkt) and c-Myc in treated tumors. FIG. 17B is set of bar graphs showing relative expression levels of EGFR in treated tumors. FIG. 17C is set of bar graphs showing relative expression levels of CK2α in treated tumors. FIG. 17D is set of bar graphs showing relative expression levels of pAkt/Akt in treated tumors. FIG. 17E is set of bar graphs showing relative expression levels of cMyc in treated tumors. Referring to FIGS. 17A-17E, significant changes were observed in relative expression levels of EGFR, CK2α, pAkt/Akt, and cMyc following treatment with NICs compared to PBS. The strongest effect was observed with AON combination. Referring to FIG. 17A, the nanodrugs' mechanism of action on brain tumor cells appears to involve inhibiting Akt phosphorylation and c-Myc expression. CK2 inhibition by a tumor-targeted nanoimmunocomjugate appears to be superior to oral inhibitor treatment, as it yielded greater mouse survival increase with nanoimmunoconjugates (89% for CK2α AON and 103% for CK2α+EGFR), vs. 59% for oral small molecule CK2 inhibitor.

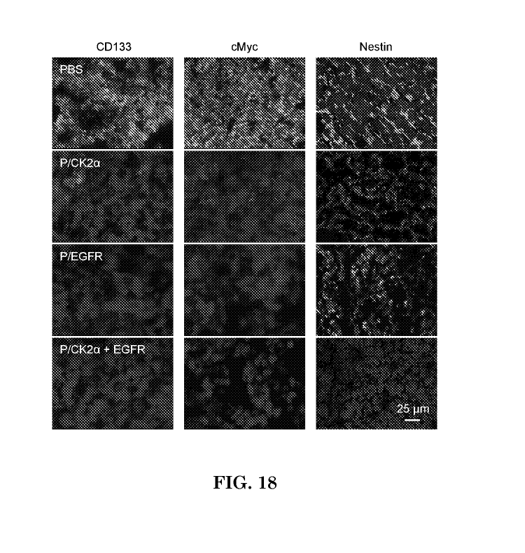

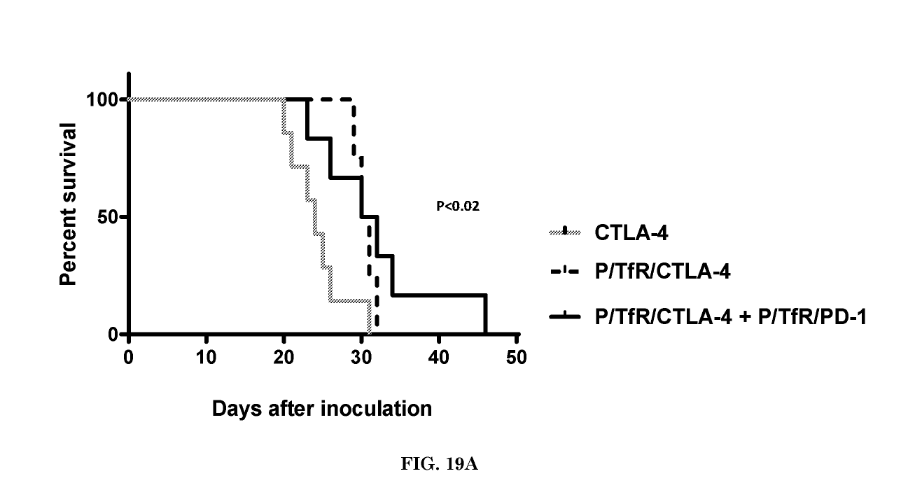

One of the clinically important problems is tumor stem cells. They not only contribute to tumor growth, but also are also more resistant to therapies than differentiated cancer cells and their survival is an important factor of tumor recurrence. For this reason, successful cancer therapies should be directed towards efficient elimination of cancer stem cells. An immunohistochemical study of treated xenogeneic LN229 tumors was conducted using several cancer stem cell markers, CD133, c-Myc and nestin. All three markers were well expressed in PBS-treated tumors. FIG. 18 is a set of photographs illustrating expression of cancer stem cell markers CD133, cMyc and Nestin in GL26 brain tumors following treatment with P/AON-CK2α, P/AON-EGFR, P/AON-EGFR/AON-CK2α and PBS. Referring to this figure, high expression of CD133, c-Myc and nestin was observed in PBS-treated tumors and its significant decrease upon treatment with nanodrugs inhibiting CK2α and EGFR. Combined inhibition of both targets abolished staining. Nuclei were counterstained with DAPI. Following immunofluorescent staining of tissue sections, it was observed, that treatment with nanodrugs bearing AON to CK2α or EGFR or especially, their combination caused a dramatic decrease in all markers expression.

The data clearly attest to the ability of the approach of blocking GBM growth and cancer stem cells by nanodrugs inhibiting the synthesis of EGFR and/or CK2.

GBM Suppression by Nanoimmunoconjugates

Nanoimmunoconjugates passing BBB were engineered with mAbs to CTLA-4 or PD-1 and used to treat mice with intracranial glioma GL26. The respective roles of systemic vs. local immunity in fighting brain tumors were examined. Mice were systemically treated 5 times with naked mAbs or nanoconjugate-attached mAbs with tumor targeting TfR mAb. Naked mAbs did not prolong animal survival vs. PBS. However, both brain tumor-targeted mAbs on nanoplatform caused significant animal survival increase. The data corroborate the assumption that stimulation of local immunity by Treg-modulating mAbs is more important for mounting anti-brain tumor response compared to systemic immune stimulation, and attest to the feasibility of this approach. In the same experiment, the concentrations of relevant cytokines in the sera of treated animals using mouse Magnetic Luminex Screening Assay (R&D Systems) were determined. It was observed that only nanopolymer-attached and tumor delivered immunomodulatory mAbs were able to dramatically boost IL-12 expression (consistent with anti-tumor response), whereas naked mAbs did not. Surprisingly, IL-2 levels were not significantly increased. For this reason, a nanoimmunodrug with IL-2 attached together with CTL-stimulating mAbs as shown in FIG. 12B were used, since IL-12 is already highly increased by the treatment.

Next determination was made on whether intratumoral CD8+ cells were increased upon brain tumor treatment, consistent with CTL activation. These cells were very rare in untreated tumors, with no significant change after systemic treatment with anti-CTLA-4 or anti-PD-1. However, it was observed that delivery of these immunomodulatory mAbs to the tumor with nanoimmunodrug resulted in increased numbers of CD8+ cells. It should be noted that CTLA-4 mAb in general caused more pronounced effect than PD-1 mAb.

The data described herein show efficacy of nanoimmunoconjugates with CK2 and EGFR AON in suppressing glioma growth and of the nanoimmunodrugs in boosting local and systemic immunity. Both treatments prolonged survival of tumor-bearing animals, making them attractive candidates for further preclinical development.

Experimental Design

Intracranial tumors were established. On day 1, mice were stereotactically injected intracranially with glioma cells at previously optimized doses. U87MG required 25×10³ cells/mouse for optimal growth, LN229, 1×10⁵ cells, and GL26 and GL261, 25×10³ cells. The use of GL26 and GL261 cells was guided by their different expression of class I and II MHC antigens: GL26 was non-immunogenic and expressed Class I MHC but not class II MHC, whereas GL261 was partially immunogenic and expressed high level of MHC I and also MHC II, B7-1 and -2, which were co-stimulatory of molecules required for T cell activation.

Tumors were grown for 3 days. On days 3, 7, 10, 14, 17, and 21 (6 treatments, as was effective in previous studies) animals were injected intravenously with the nanoimmunojugates shown in Table 1-4 and control agents. The standard dose of a nanoimmunoconjugate dose was 5.0 mg/kg by AON, and 3-10 mg/kg of CTLA-4 and PD-1. Each group consisted of 8 mice as approved by the Cancer Center Biostatistics core. Animals were sacrificed when they developed neurological abnormalities. General controls were as follows: 1) 8 mice were euthanized on day 30 without any treatment to obtain normal control tissue; 2) 8 mice per treatment group were injected with naked PMLA or PBS, or nanoconjugate with scrambled AONs; isotype control IgG on nanoimmunodrug was used in experiments with immunostimulating antibodies; Control for systemic vs. local immune stimulation included standard of care naked anti-CTLA-4 and/or anti-PD-1 antibodies, to compare efficacy with BBB-passing nanoimmunodrug. Naked AONs do not pass through cell membranes in vivo and were not used as a control. Outcome measures Excised tumors were analyzed by H&E staining, measurement of size and expression of target and lymphocyte markers (CD8 and CD4) by Western blot and immunohistochemistry. To examine the mechanisms of drug action and immune stimulation, phosphorylation of Akt, STAT5, ERK1/2, and the extent of apoptosis by cleaved PARP were evaluated as described (Inoue et al., 2012, PLoS One, 7: e31070, which is incorporated herein by reference as if fully set forth). Cytokine levels (IL-2 and IL-12) were determined in animal sera.

Cancer stem cells were detected by immunostaining and FACS analysis (in Cedars-Sinai core) after nanoimmunoconjugate treatment and their marker expression were correlated with tumor size and survival of glioma bearing animas. Cancer stem cells induced immunosuppression by expressing program cell death ligand-1 (PD-L1) and TGF-81, as well as by inhibiting T cell proliferation, inducing T cell apoptosis and enhancing Treg function.

A combination of AON to EGFR and CK2 with immunostimulatory antibodies or anti-tumor cytokines produced a synergistic effect.

Evaluation of Tumor Size

H&E staining is performed and tumor diameter was measured in surface, and in the center of the tumor. The tumor volume (V) was evaluated by the formula V=π/6×a²×b, where a is the short axis and b is the long axis.

Expression of Molecular Targets

Specific antibodies were used to detect the expression of CK2, EGFR, CTLA-4, PD-1, CD8, and CD4 in tumors after treatments in comparison with control animals receiving scrambled AON-nanodrug or PBS. After sectioning, the rest of the tumor tissues and adjacent tissue at the distance 2-8 mm were scooped from the OCT blocks for protein extraction. Subsequent Western analysis determined semi-quantitatively phosphorylation state of Akt, STAT5, ERK1/2, and cleaved PARP for apoptosis.

Cytokine Measurements

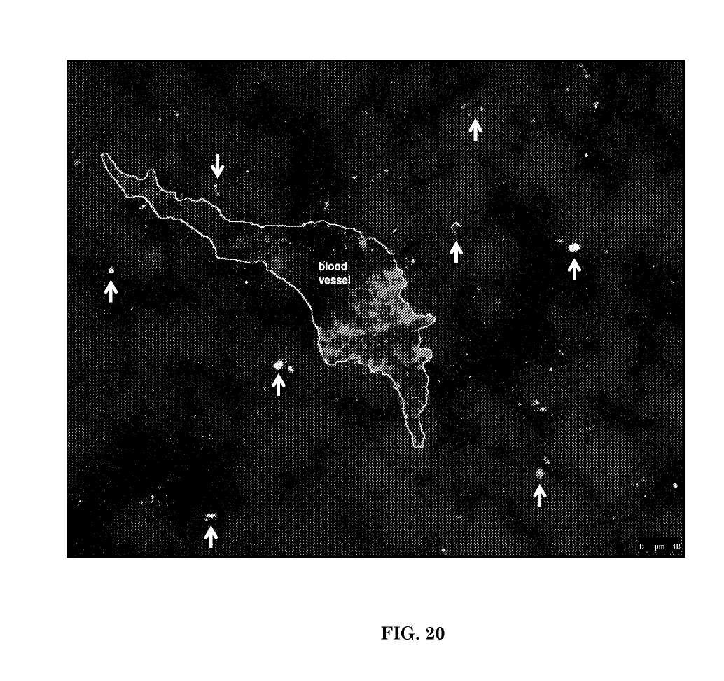

Cytokine levels (e.g., IL-2 and IL-12) were determined in animal sera using Luminex assay as was described and illustrated on FIG. 20.

Statistical analysis was done by ANOVA for multiple groups.

Pharmacokinetic and Toxicological Studies of Nanoconjugates

For immunogenicity tests, the approved CTLA-4 and PD-1 antibodies that react with human antigens were used, to eliminate possibly augmented immune response in animals due to their action.

Experimental Design

For assessing drug half-life and tissue distribution, radioactively labeled nanoimmunoconjugates were used. Blood samples and tissue homogenates at various times were prepared to measure radioactivity distribution over molecular weight by sec-HPLC indicating nanoimmunodrugs degradation, and by scintillation counting. Urine samples were also analyzed. Quality and stability in PBS and human plasma, as well as clinical biochemistry as a possible toxicity indicator were determined. Acute toxicity in mice, specific pharmacokinetics and ADME (absorption, distribution, metabolism and excretion) of the lead compound, as well as the maximum tolerated dose (MTD) were conducted in the laboratory.

Drug Half-Life and Tissue Distribution Study

This was performed using polymer-conjugated radioactive tracer. Groups of experimental and control tumor-bearing (n=5/group) were injected intravenously with ¹²⁵I-labeled nanoimmunodrugs with AONs to CK2 and EGFR and pertinent mAbs in 150 μl volume at a dose determined from above. Half-life was determined by clearance (CL) and volume of distribution (Vd) and the relationship is described by the following equation: t_{1/2}=logₑ 0.5 Vd/CL. Blood and urine samples, and tissue homogenates times were assessed at 10 min, 3, 12, 24, 48, and 72 hr for associated label distribution over molecular weight by sec-HPLC indicating nanoimmunodrug degradation. Labeled nanoimmunoconjugates were detected in organs using scintillation counting. Microscopic imaging was done to detect Alexa Fluor 680-labeled nanoimmunodrugs. The study was repeated 3 times.

Control of Nanoimmunodrugs Quality and Stability

(1) Quality control: In human plasma, ELISA assay was used to detect activities of different mAbs after 24 hr incubation at 37° C. (2) Stability control: The ester bonds of PMLA were mostly broken during storage. Solutions of nanoimmunoconjugates were stored at −20° C. They remained active after 3, 6, 12, and 15 months of storage. Lyophilized nanoimmunodrugs were stable at −20° C. for 2 years.

Immunological Characterization

Standard assay tests have been developed and established and used to characterize nanoimmunoconjugates. Endotoxin removal was assayed by rabbit pyrogenic tests in a GLP-certified laboratory. Clinical biochemistry testing was done according to the standard procedures for biochemical clinical tests in experimental and control animals: Clinical manifestations: Slow moving, loss of interest, activities (hyper/hypo). Nutrition. Neurological score: Grade 1: tail weakness or tail paralysis; Grade 2: hind leg/limb paresis or hemiparesis; Grade 3: hind leg/limb hemiparalysis; Grade 4: complete paralysis (tetraplegia), moribund stage or death. Blood biochemistry: transaminase (AST, ALT)-liver function; bilirubin (direct, indirect); creatinin; blood urea nitrogen; blood: white blood cells; red blood cells; platelets; hemoglobin; inflammation: C-reactive peptide.

Acute Toxicity Study and Repeated I.V. Dose Toxicity Study in Mice

There were 3 dose levels (high, medium and low) with 5 animals/sex/dose for a total number of 30. All animals received a single intravenous dose. Body weights were taken once weekly; clinical signs were taken twice on the day of dosing and once daily thereafter, with a mortality check twice daily at least 6 hours apart. Necropsy was done on day 15. If any gross lesions were found, these tissues were collected in formalin and histopathology was performed. For monkeys, there were 2 dose levels plus a control group. There was 1 animal/sex/dose for a total of 6 animals (FDA-acceptable), which was administered an I.V. dose on days 1, 4, 8 and 11. Body weight was taken twice a week starting in the latter half of week 1 and at termination. Consumption of food was measured semi-quantitatively. Mortality check was done twice daily at least 6 hr apart. Clinical signs were checked daily, ˜1-2 hours after dosing. Clinical observations (physical exams) were done weekly starting at week 1. Clinical pathology was done once during pretest and at termination on day 15 for all animals. PK samples were collected on day 1 and day 11 (after the 4^{th} dose). Necropsy was performed on day 15 for all survivors, and for all found dead animals. Organ weights were taken for major organs; a bone marrow smear was prepared from the rib and evaluated. Clinical pathology and histopathology tests were conducted.

Statistical Analysis

For toxicology studies, ANOVA tests were conducted on body weight changes, food consumption, hematology, clinical pathology, cardiology measurements, and organ weight data. If a significant F ratio was obtained (p≤0.05), Dunnett's t test was used for pair-wise comparisons to the control group. Duncan's multiple range test for pair-wise comparisons was alternatively used. Because clinical chemistry and hematology parameters change as a function of age, the measurements were statistically analyzed at discreet points in time as recommended by a Joint Task Force of the American Association for Clinical Chemistry. Frequency data such as incidence of mortality, gross necropsy and tissue morphology observations were compared by Fisher's exact test or Chi-square analyses. SAS, SPSS and BMPD statistical analyses programs were also available.

Treatment of Mice Inoculated with GL261 Cells

20,000 mice glioma cells GL261 were inoculated intracranially. In 5 days after glioma cell inoculation, a group of mice were treated with free antibody CTLA-4, (10 mg/kg), P-CTLA-4/msTfR, P-PD-1/mTfR and combination of P-CTLA-4/msTfR+P-PD-1/msTfR were administered twice a week, with a total of five I.V. injections.

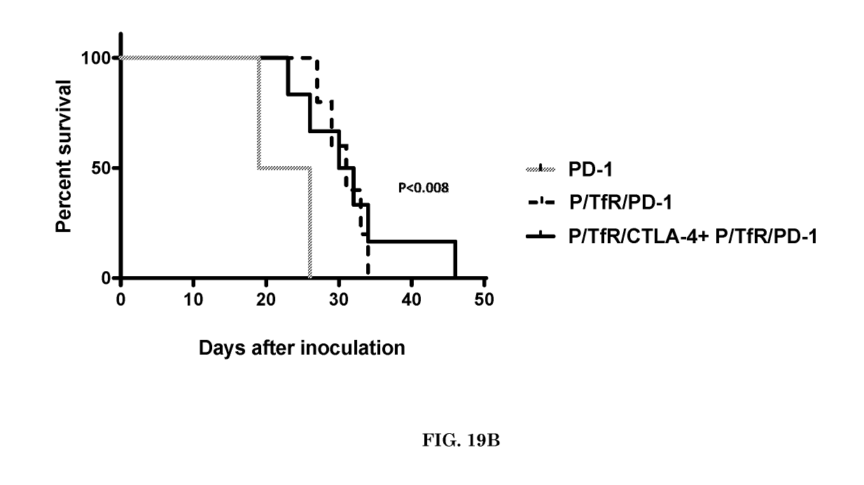

20,000 mice glioma cells GL261 were inoculated intracranially. In 5 days after glioma cell inoculation group of mice were treated with free antibody CTLA-4, (10 mg/kg), P-CTLA-4/msTfR, P-PD-1/mTfR and combination of P-CTLA-4/msTfR+P-PD-1/msTfR were administered twice a week, with a total of five I.V. injections. FIGS. 19A-19B are Kaplan Meier curves illustrating animal survival after treatment with nanoimmunoconjugates. FIG. 19A illustrates animal survival after treatments with CTLA-4 mAB, P/TfR/CTLA-4 mAb and a combination of P/TfR/CTLA-4 and P/TfR/PD-1. FIG. 19B illustrates animal survival after treatments with PD-1 mAB, P/TfR/PD-1 mAb and a combination of P/TfR/CTLA-4 and P/TfR/PD-1. P refers to Polymer. The figure illustrates animal survival following activation of general and tumor local immune system after treatment with nanoimmunoconjugates compared to free mAbs. Mice bearing brain tumors GL.261 were treated with Abs against check points' inhibitors CTLA-4 mAb and PD-1 mAb delivered into the brain tumors as part of the nanoimmunoconjugates, or free CTLA-4 and PD-1 as IgG1 antibody. It was observed that free CTLA-4 and PD-1 as IgG1 antibody do not cross BBB. In contrast, the nanoimmunoconjugates were crossing BBB and activated brain tumor immune system. Referring to FIG. 19A, it was observed that the animal survival rate was higher after treatment with a combination of P/TfR/CTLA-4 mAb+P/TfR/PD-1 mAB, P<0.02, compared to treatment with only one nanoimmunoconjugate P/TfR/CTLA-4 mAb, or CTLA-4 mAB. Referring to FIG. 19B, it was observed that the animal survival rate was higher after treatment with a combination of P/TfR/CTLA-4 mAb+P/TfR/PD-1 mAB, P<0.02P/TfR/PD-1 mAB, P<0.008 compared to treatment with only P/TfR/PD-1 or PD-1 mAB. FIG. 20 is a photograph illustrating the nanoimmunoconjugate P/a-CTLA-4/PD-1/TfR crossing BBB (white arrows). Referring to this figure, the blood vessel contour is outlined. White dots marked by the arrows are nanoimmunoconjugate accumulations providing evidence of BBB crossing. The nanoimmunoconjugate was synthesized using infrared dye, Rhodamine.

Flow Cytometry Markers were Studied:

When mice reached the humane endpoints, they were euthanized and tumors were harvested and used to analyze T-cell population by flow cytometry. CD3 was used to identify T-cells; CD4, CD8, and FOXP3 were used to identify T-effectors and T-regulators cells within the T-cell population; CD69 and IFNγ were used to measure CD4+ and CD8+ T-cells activation; PD1 and CTLA4 were used to measure expression of therapeutic targets by CD4+ and CD8+ T-cells.

Flow Cytometry Analysis Results in Tumor Tissue

The total number of CD4+ T-cells was reduced in animals treated with polymer/anti-PD1 and combination treatment compared to free antibody anti-PD1. Although there was no statistical significance, the fraction of Tregs (CD4+FOXP3+) was also reduced by all polymer conjugated treatments compared to free antibody treatments. Similarly, CD8+ T-cells were increased in number in mice treated with polymer conjugated antibodies compared to free antibody, but the difference did not reach statistical significance.

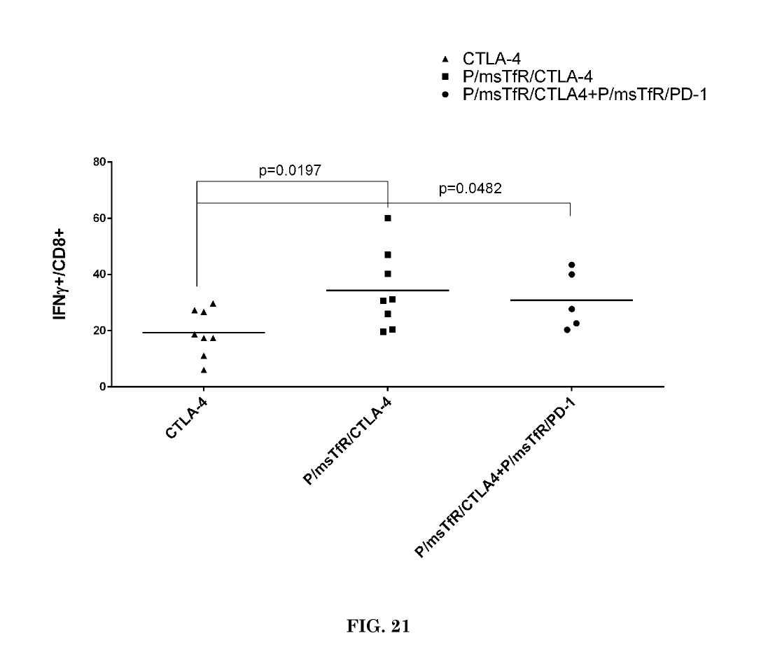

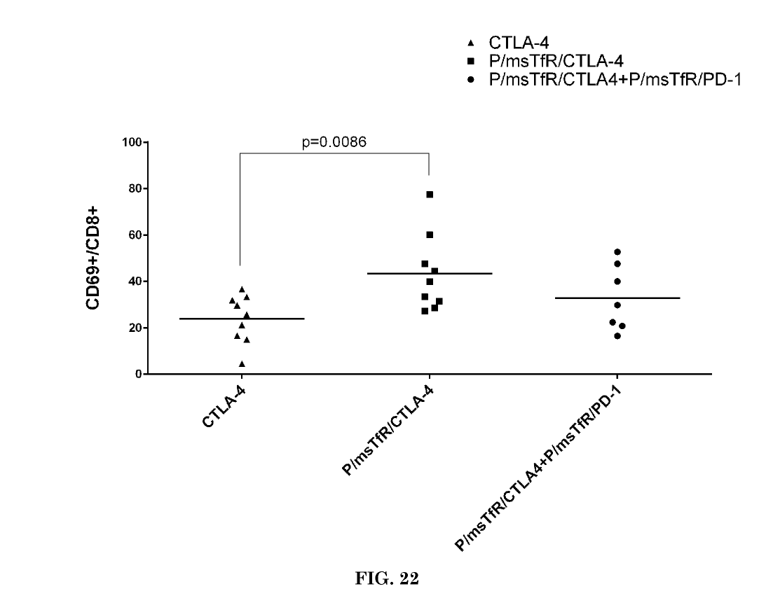

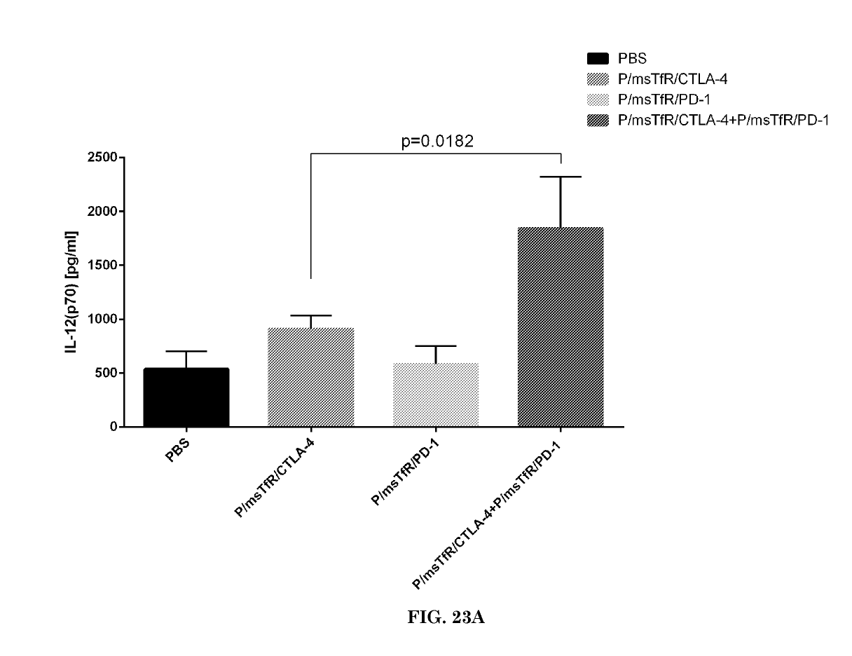

FIG. 21 is a scatter plot illustrating analysis of IFNγ/CD8+ cells following treatments of animals with CTLA-4 mAb, P/msTfR/CTLA-4 and P/msTfR/CTLA-4+P/msTfR/PD-1. Referring to this figure, the activation of tumor local immune system as was observed after treatment with nanoimmunoconjugates P/msTfR/CTLA-4 and P/msTfR/CTLA-4+P/msTfR/PD-1. FIG. 22 is a scatter plot illustrating analysis of CD69+/CD8+ cells following treatments of animals with CTLA-4 mAb, P/msTfR/CTLA-4 and P/msTfR/CTLA-4+P/msTfR/PD-1. It was observed that polymer conjugated antibodies did not produce a significant increase in CD69 and IFNγ expression in CD4+ T-cells. Instead, activation of CD8+ T-cells was significantly increased by polymer conjugated anti-CTLA4 antibody and combination therapy (co-injection of two conjugates: P/TfR/CTLA-4+P/TfR/PD-1), compared to free CTLA4 antibody therapy.

Polymer conjugated anti-PD1 antibody and combination treatment produced a decrease in PD1 expression in CD4+ T-cells, although not statistically significant, compared to free an anti-PD1 antibody. Moreover, animals treated with polymer conjugated anti-PD1 antibody and combination treatment show a significant decrease in PD1 expression by CD8+ cells, both compared to anti-PD1 antibody and anti-CTLA4 antibody treatments. CTLA4 expression on both CD4+ and CD8+ T-cells does not seem to be affected by polymer conjugated treatments compared to free antibody treatments.

Multiplex Cytokine Assay

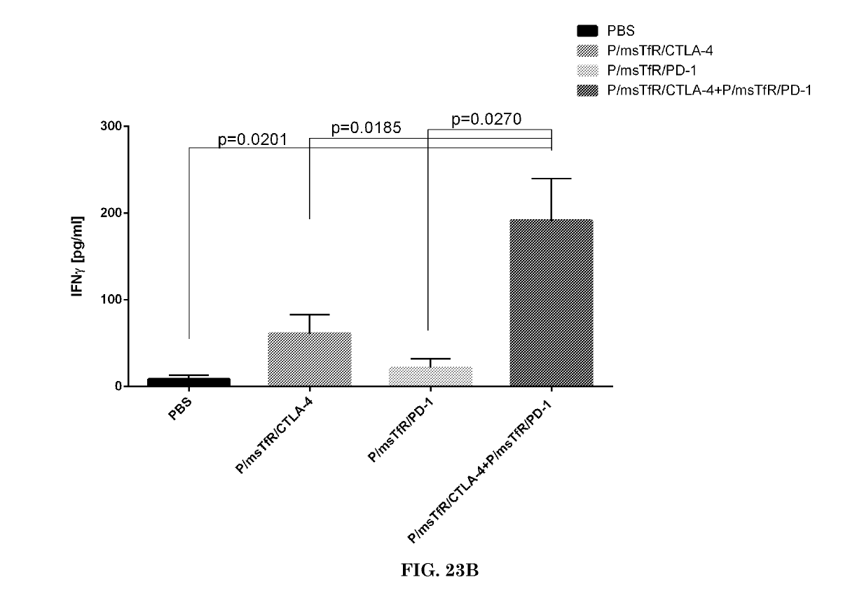

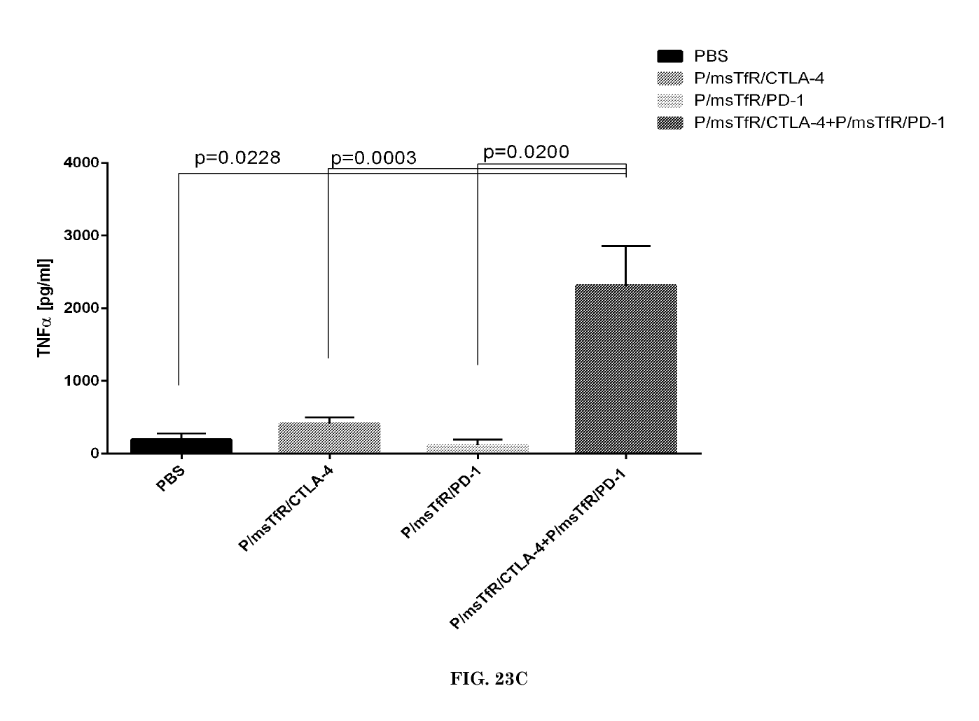

Serum from C57/B16 mice bearing GL261 glioblastoma in brain were used to measure cytokine levels using a BioRad Bioplex assay. Mice were administered with three I.V. injections alternatively with PBS, polymer conjugated anti-PD1 antibody, polymer conjugated anti-CTLA4 antibody, or a combination of the last two. Serum was harvested 24 hours after the third treatment. FIGS. 23A-23C are bar graphs illustrating cytokine levels in serum from C57/BI6 mice bearing GL26 glioma following treatments with P/msTfR/CTLA-4, P/msTfR/PD-1 and P/msTfRCTLA-4+P/msTfR/PD-1. FIG. 23A illustrates IL-12(p70) levels. FIG. 23B illustrates IFNγ levels. FIG. 23C illustrates TNFα levels.

A clear trend was visible in treatments, where cytokine expression was increased in animals treated with polymer conjugated antibodies, and, in particular, with combination therapy compared to PBS treated mice. Specifically, the combination therapy produced a statistically significant increase in the expression of IL-18, IL-2, IL-4, IL-5, IL-6, IL-10, IL-12(p70), IFNγ, and TNFα compared to the other treatments. Increase in cytokine levels denotes activation of the immune system and in particular the T-cell population.

The results indicated activation of T-cell population locally at the tumor level in brain following treatment with polymer conjugated antibodies anti-PD1 and anti-CTLA4. The same activation was not triggered by the same antibodies when non conjugated to the PMLA polymer. Furthermore, the data show activation of the immune system in the form of an increase in cytokine levels both at systemic level in serum and locally in the tumor inside the brain.

Advantages of the Nanoimmunoconjugates

Compared to existing nanomedicines, experimental and already used in clinic (Doxil, Abraxane, etc.) the nanoimmunoconjugates disclosed herein have several significant advantages, especially for breast cancer and brain tumor treatment. They can pass through the blood brain barrier (BBB) and the blood tissue barrier (BTB) not by slow and inefficient EPR effect, but by active transcytosis through tumor vasculature without losing their payload. Covalent binding of all moieties to the polymalic acid-based molecular scaffold ensures delivery to the tumor site without leakage common to nanoparticles and liposomes. Dual targeting of tumor vasculature and cancer cells ensures specific drug delivery to its intended target without appreciable effect on adjacent normal tissues. They are fully biodegradable and non-toxic in animals. They are the nanodrugs capable of stimulating local tumor immunity. These significant advantages make the nanoimmunoconjugates disclosed herein very attractive drugs for treating brain cancer and breast cancer.

The hypothesis was to activate general immune system together with local tumor immune system by delivering these antibodies anti-CTLA-4 and/or anti-PD-1 as part of conjugates. The nanoimmunoconjugates were able to cross tumor endothelial systems for brain and breast. It was confirmed by the experimental data that treatment of primary brain and breast cancers and metastases of breast cancers to the brain was significantly better than treatment with free anti-CTLA-4 and anti-PD-1. For brain tumors, the treatment is not effective in clinic because these antibodies do not cress the blood-brain barrier. Thus, the activation of general immune response is not enough for the brain tumor treatment. Breast cancer is much better treated with nanoimmunoconjugates administrated systemically, which is shown by in vivo treatment data.

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

1. A nanoimmunoconjugate comprising:
a polymalic acid-based molecular scaffold,
at least one targeting ligand,
at least one anti-tumor immune response stimulator, and
at least one anti-cancer agent,
wherein the targeting ligand, the anti-tumor immune response stimulator and the anti-cancer agent are covalently linked to the polymalic acid-based molecular scaffold.

2. The nanoimmunoconjugate of claim 1, wherein the anti-tumor immune response stimulator is selected from the group consisting of: an antisense oligonucleotide (AON), an siRNA oligonucleotide, an antibody, a polypeptide, an oligopeptide and a low molecular weight drug.

3. The nanoimmunoconjugate of claim 2, wherein the anti-tumor immune response stimulator is an antibody.

4. The nanoimmunoconjugate of claim 3, wherein the anti-tumor immune response stimulator is selected from the group consisting of: an antibody against PD-1, an antibody against PD-L1, an antibody against PD-L2, an antibody against CTLA-4, or a combination thereof.

5. The nanoimmunoconjugate of claim 2, wherein the anti-tumor immune response stimulator is an antisense oligonucleotide or an siRNA comprising a sequence complementary to a sequence contained in an mRNA transcript of an immune checkpoint protein.

6. The nanoimmunoconjugate of claim 5, wherein the antisense oligonucleotide is a morpholino antisense oligonucleotide.

7. The nanoimmunoconjugate of claim 5, wherein the antisense oligonucleotide comprises a sequence with at least 90% identity to a sequence selected from the group consisting of SEQ ID NOS: 4-7.

8. The nanoimmunoconjugate of claim 1, wherein the anti-tumor immune response stimulator is an inhibitor of an immune checkpoint protein.

9. The nanoimmunoconjugate of claim 1, wherein the anti-tumor immune response stimulator is an immunostimulatory cytokine.

10. The nanoimmunoconjugate of claim 9, wherein the cytokine is IL-2 or IL-12.

11. The nanoimmunoconjugate of claim 1, wherein the anti-cancer agent is selected from the group consisting of: an antisense oligonucleotide, an siRNA oligonucleotide, an antibody, a polypeptide, an oligopeptide and a low molecular weight drug.

12. The nanoimmunoconjugate of claim 11, wherein the anti-cancer agent is the antisense oligonucleotide comprising a sequence with at least 90% identity to a sequence selected from the group consisting of SEQ ID NO: 1, 2 and 8.

13. The nanoimmunoconjugate of claim 11, wherein the anti-cancer agent is an antisense oligonucleotide or an siRNA comprising a sequence complementary to a sequence contained in an mRNA transcript of a human epidermal growth factor receptor (HER), or the serine-threonine protein kinase (CK2).

14. The nanoimmunoconjugate of claim 11, wherein the anti-cancer agent is an antisense oligonucleotide comprising a sequence complementary to a sequence with at least 90% identity to the sequence of SEQ ID NO: 3.

15. The nanoimmunoconjugate of claim 11, wherein the anti-cancer agent is an anti-HER2/neu antibody.

16. The nanoimmunoconjugate of claim 15, wherein the anti-HER2/neu antibody is Herceptin®.

17. The nanoimmunoconjugate of claim 1, wherein the nanoimmunoconjugate comprises at least two different anti-cancer agents covalently linked to the polymalic acid-based molecular scaffold.

18. The nanoimmunoconjugate of claim 1, wherein the targeting ligand binds specifically to a vasculature protein in a tumorigenic cell or cancer cell.

19. The nanoimmunoconjugate of claim 18, wherein the vasculature protein comprises a transferrin receptor protein.

20. The nanoimmunoconjugate of claim 1, wherein the targeting ligand is an antibody.

21. The nanoimmunoconjugate of claim 1, wherein the nanoimmunoconjugate further comprises a PK modulating ligand covalently linked with the polymalic acid-based molecular scaffold.

22. The nanoimmunoconjugate of claim 21, wherein the PK modulating ligand is polyethylene glycol (PEG).

23. The nanoimmunoconjugate of claim 1, wherein the nanoimmunoconjugate further comprises an endosomolytic ligand covalently linked with the polymalic acid-based molecular scaffold.

24. The nanoimmunoconjugate of claim 23, wherein the endosomolytic ligand comprises a plurality of leucine or valine residues.

25. The nanoimmunoconjugate of claim 24, wherein the endosomolytic ligand is Leu-Leu-Leu (LLL).

26. The nanoimmunoconjugate of claim 1, wherein the nanoimmunoconjugate further comprises an imaging agent covalently linked with the polymalic acid-based molecular scaffold.

27. A pharmaceutically acceptable composition comprising an nanoimmunoconjugate of claim 1 and a pharmaceutically acceptable carrier or excipient.

28. A method for treating cancer in a subject comprising:
providing a nanoimmunoconjugate comprising:
a polymalic acid-based molecular scaffold,
at least one targeting ligand,
at least one anti-tumor immune response stimulator, and
at least one anti-cancer agent,
wherein the targeting ligand, the anti-tumor immune response stimulator and the anti-cancer agent are covalently linked to the polymalic acid-based molecular scaffold, and
administering a therapeutically effective amount of the nanoimmunoconjugate to a subject.

29. The method of claim 28, wherein the anti-tumor immune response stimulator is selected from the group consisting of: an antisense oligonucleotide (AON), an siRNA oligonucleotide, an antibody, a polypeptide, an oligopeptide and a low molecular weight drug.

30. The method of claim 28, wherein the anti-tumor immune response stimulator is at least one antibody selected from the group consisting of: an antibody against PD-1 antibody, an antibody against PD-L1, an antibody against PD-L2, an antibody against CTLA-4, or a combination thereof.

31. The method of claim 29, wherein the anti-tumor immune response stimulator is an antisense oligonucleotide or an siRNA comprising a sequence complementary to a sequence contained in an mRNA transcript of an immune checkpoint protein.

32. The method of claim 31, wherein the antisense oligonucleotide is a morpholino antisense oligonucleotide comprising a sequence with at least 90% identity to a sequence selected from the group consisting of SEQ ID NOS: 4-7.

33. The method of claim 28, wherein the anti-tumor immune response stimulator is an inhibitor of an immune checkpoint protein.

34. The method of claim 28, wherein the anti-tumor immune response stimulator is an immunostimulatory cytokine selected from the group consisting of IL-2 and IL-12.

35. The method of claim 28, wherein the anti-cancer agent is selected from the group consisting of: an antisense oligonucleotide, an siRNA oligonucleotide, an antibody, a polypeptide, an oligopeptide and a low molecular weight drug.

36. The method of claim 35, wherein the anti-cancer agent is the antisense oligonucleotide comprising a sequence with at least 90% identity to a sequence selected from the group consisting of SEQ ID NO: 1, 2 and 8.

37. The method of claim 35, wherein the anti-cancer agent is an antisense oligonucleotide or an siRNA comprising a sequence complementary to a sequence contained in an mRNA transcript of a human epidermal growth factor receptor (HER), or the serine-threonine protein kinase (CK2).

38. The method of claim 35, wherein the anti-cancer agent is an antisense oligonucleotide, and comprises a sequence complementary to a sequence with at least 90% identity to the sequence of SEQ ID NO: 3.

39. The method of claim 35, wherein the anti-cancer agent is an antibody, and wherein the antibody is an antibody against HER2/neu.

40. The method of claim 28, wherein the nanoimmunoconjugate comprises at least two different anti-cancer agents covalently linked to the polymalic acid-based molecular scaffold.

41. The method of claim 28, wherein the targeting ligand binds specifically to a vasculature protein in a tumorigenic cell or cancer cell.

42. The method of claim 28, wherein the nanoimmunoconjugate further comprises a PK modulating ligand covalently linked with the polymalic acid-based molecular scaffold.

43. The method of claim 28, wherein the nanoimmunoconjugate further comprises an endosomolytic ligand covalently linked with the polymalic acid-based molecular scaffold.

44. The method of claim 28, wherein the step of administering results in treating, reducing the severity or slowing the progression of cancer in the subject.

45. The method of claim 44, wherein the cancer is a primary cancer, a metastatic cancer, or both.

46. The method of claim 44, wherein the cancer is a primary HER2+ breast cancer, triple negative breast cancer (TNBC) or their metastasis to the brain.

47. The method of claim 44, wherein the cancer is glioma or glioblastoma.

48. A method for treating cancer in a subject, comprising:
providing a nanoconjugate comprising a polymalic acid-based molecular scaffold and at least one targeting ligand and at least one anti-cancer agent covalently linked to the scaffold; and
co-administering a therapeutically effective amount of an anti-tumor immune response stimulator and a therapeutically effective amount of the nanoconjugate to a subject.

49. The method of claim 48, wherein the anti-tumor immune response stimulator is selected from the group consisting of: an antisense oligonucleotide (AON), an siRNA oligonucleotide, an antibody, a polypeptide, an oligopeptide and a low molecular weight drug.

50. The method of claim 49, wherein the anti-tumor immune response stimulator is an antibody, and wherein the antibody is selected from the group consisting of: an antibody against PD-1 antibody, an antibody against PD-L1, an antibody against PD-L2, an antibody against CTLA-4, or a combination thereof.

51. The method of claim 49, wherein the anti-tumor immune response stimulator is an antisense oligonucleotide or an siRNA comprising a sequence complementary to a sequence contained in an mRNA transcript of an immune checkpoint protein.

52. The method of claim 51, wherein the anti-tumor immune response stimulator is an antisense oligonucleotide and comprises a sequence with at least 90% identity to a sequence selected from the group consisting of SEQ ID NOS: 4-7.

53. The method of claim 48, wherein the anti-tumor immune response stimulator is an inhibitor of an immune checkpoint protein.

54. The method of claim 48, wherein the anti-tumor immune response stimulator is an immunostimulatory cytokine, and the immunostimulatory cytokine is one of IL-2 or IL-12.

55. The method of claim 48, wherein the anti-cancer agent is selected from the group consisting of: an antisense oligonucleotide, an siRNA oligonucleotide, an antibody, a polypeptide, an oligopeptide and a low molecular weight drug.

56. The method of claim 55, wherein the anti-cancer agent is the antisense oligonucleotide, and comprises a sequence with at least 90% identity to a sequence selected from the group consisting of SEQ ID NO: 1, 2 and 8.

57. The method of claim 55, wherein the anti-cancer agent is an antisense oligonucleotide or an siRNA comprising a sequence complementary to a sequence contained in an mRNA transcript of a human epidermal growth factor receptor (HER), or the serine-threonine protein kinase (CK2).

58. The method of claim 55, wherein the anti-cancer agent is an antisense oligonucleotide and comprises a sequence complementary to a sequence with at least 90% identity to the sequence of SEQ ID NO: 3.

59. The method of claim 55, wherein the anti-cancer agent is an anti-HER2/neu antibody.

60. The method of claim 48, wherein the targeting ligand binds specifically to a vasculature protein in a tumorigenic cell or cancer cell.

61. The method of claim 48, wherein the nanoconjugate further comprises a PK modulating ligand covalently linked with the polymalic acid-based molecular scaffold.

62. The method of claim 48, wherein the nanoconjugate further comprises an endosomolytic ligand covalently linked with the polymalic acid-based molecular scaffold.

63. The method of claim 48, wherein the cancer is a primary cancer, a metastatic cancer, or both.

64. The method of claim 63, wherein the cancer is a primary HER2+ breast cancer, triple negative breast cancer (TNBC) or their metastasis to the brain.

65. The method of claim 48, wherein the method further comprises co-administering an additional therapeutic agent to the subject.

66. The method of claim 48, wherein the method further comprises co-administering one or more additional anti-cancer therapy to the subject.

67. The method of claim 66, wherein the additional anti-cancer therapy is selected from the group consisting of surgery, chemotherapy, radiation therapy, thermotherapy, immunotherapy, hormone therapy, laser therapy, anti-angiogenic therapy, and any combinations thereof.

68. The method of claim 48, wherein the subject is a mammal.

69. The method of claim 68, wherein the mammal is selected from the group consisting of: a rodent, an experimental human-breast tumor-bearing nude mouse and a human.


SEQUENCE LISTING (8 sequences)

SEQ ID NO: 1 - DNA, 24 bp, Artificial Sequence
  Synthetic construct, AON HER2/neu
catggtgctcactgcggctccggc

SEQ ID NO: 2 - DNA, 24 bp, Artificial Sequence
  Synthetic construct, AONhuman&mouse CK2
cggacaaagctggacttgatgttt

SEQ ID NO: 3 - DNA, 24 bp, Artificial Sequence
  Synthetic construct, CK2 consensus seq
cggacaaagctggacttgatgttt

SEQ ID NO: 4 - DNA, 25 bp, Artificial Sequence
  Synthetic construct, AON CTLA-4_1
gtcctcagggagcagagtaaaaccc

SEQ ID NO: 5 - DNA, 25 bp, Artificial Sequence
  Synthetic construct, AON CTLA-4_2
tccagaagccttgagatgtgtttga

SEQ ID NO: 6 - DNA, 25 bp, Artificial Sequence
  Synthetic construct, AON PD-1_1
tacctgccggacccacatgcccaga

SEQ ID NO: 7 - DNA, 25 bp, Artificial Sequence
  Synthetic construct, AON PD-1_2
cctggcagtgtcgccttcagtagca

SEQ ID NO: 8 - DNA, 25 bp, Artificial Sequence
  Synthetic construct AON EGFR
tcgctccggctctcccgatcaatac